(19) United States
(12) Patent Application Publication
(10) Pub. No.: US 2020/0043598 A1
(43) Pub. Date: Feb. 6, 2020

(54) SYSTEM AND METHOD FOR INTERPRETING AN ECHOCARDIOGRAM

(71) Applicant: Richard Marcus, West Des Moines, IA (US)

(72) Inventor: Richard Marcus, West Des Moines, IA (US)

(21) Appl. No.: 16/516,378

(22) Filed: Jul. 19, 2019

Related U.S. Application Data
(60) Provisional application No. 62/712,542, filed on Jul. 31, 2018.

(51) Int. Cl. G16H 30/20; A61B 8/08

(52) U.S. Cl. CPC G16H 30/20; A61B 8/0883

(57) ABSTRACT

This disclosure relates to healthcare. More specifically, and without limitation, this disclosure relates to a system and method of interpreting the data provided by echocardiograms to assist practitioners.

### CROSS REFERENCE TO RELATED APPLICATIONS

This application claims priority to U.S. Provisional Application No. 62/712,542 which was filed on Jul. 31, 2018, the entirety of which is incorporated herein fully by reference.

### FIELD OF THE DISCLOSURE

This disclosure relates to healthcare. More specifically, and without limitation, this disclosure relates to a system and method of interpreting the data provided by echocardiograms to assist practitioners.

### BACKGROUND OF DISCLOSURE

This disclosure relates to the healthcare field. More specifically, this disclosure relates to cardiovascular diseases and prevention through the use and interpretation of an echocardiogram. Cardiovascular diseases are the leading cause of death globally. Cardiovascular diseases resulted in almost 18 million deaths in 2015. Cardiovascular diseases are heart conditions that includes diseased blood vessels, structural heart problems, functional heart problems, blood clot related issues, among many other ailments. Cardiovascular diseases are largely preventable through proper diet, exercise, patient monitoring, early diagnosis and treatment.

One mode of diagnosing and monitoring a patient's heart and/or cardiovascular system is through the performance of an echocardiogram. An echocardiogram (or echo) is a test that uses high frequency sound waves (ultrasound) to make pictures of a patient's heart. This test is also commonly referred to as echocardiography or diagnostic cardiac ultrasound.

Doctors use an echocardiogram to look at the heart structure of a patient as well as to check how well a patient's heart is functioning. An echocardiogram helps a doctor, nurse, or other healthcare professional find out the size and shape of a patient's heart as well as the thickness and movements of the walls of the heart. In addition, echocardiograms also provide information which helps a healthcare professional decipher how a heart is moving, the pumping strength of a heart, and other features and functions of the heart. Furthermore, an echocardiogram can provide enough data to tell a healthcare professional if the heart valves are working correctly.

Other data provided from an echocardiogram, if interpreted correctly, can guide a healthcare professional to interpret if blood is leaking through the heart valves (regurgitation), if the heart valves are too narrow (stenosis), and even if there is a tumor or infectious growth in the heart or around the heart valves. Furthermore, an echocardiogram can provide detailed data to a healthcare professional. An echocardiogram can even provide a healthcare with data related to the outer lining of the heart (the pericardium). An echocardiogram can even provide data about the blood vessels surrounding the heart that may be critical to catching or diagnosing cardiovascular diseases. For example, an echocardiogram can provide data related to the large blood vessels that enter and leave the heart. Another example is that an echocardiogram can provide data identifying blood clots in and near the chambers of the heart. Another example is that an echocardiogram can even detect holes between the chambers of a heart. An echocardiogram can provide an enormous amount of data to a healthcare professional which can aid, or if interpreted improperly hinder, the professional's diagnosis and/or treatment of the patient.

To properly interpret the complex and voluminous results from an echocardiogram the healthcare professional must understand and interpret large amounts of data. In addition, it is important that in clinical practice a healthcare professional understand the underlying physiology and patient's clinical status, including patient history, prior conditions, prior procedures, prior test results and other complicating health factors. Also, knowing the inherent limitations of the echocardiogram will aid the healthcare professional interpret the results and avoid misdiagnosis. Therefore, it is important for healthcare professionals to combine an understanding of the patient's current clinical status, historical data, and current echocardiogram data to provide an optimum interpretation. Due to increased time and cost pressures on healthcare professionals, healthcare professional must digest and interpret this massive amount of data in a minimum amount of time.

On average, an echocardiogram only takes about 30 to 45 minutes to perform. Yet, the amount of data an echocardiogram can provide is extensive. A healthcare professional must access, assess and interpret the echocardiogram data quickly. In some cases, a healthcare professional is expected to digest, review, and diagnose results of an echocardiogram in only a matter of minutes. Understanding the patient's history, assessing the echocardiogram, and quantitatively collating this information with the patient's overall current status can be overwhelming. It is no surprise that an enormous amount of literature has been produced on the proper evaluation of echocardiograms. Data management can be a problem in any field, but it is especially true in the cardiovascular field when mere millimeter measurements can change a diagnosis.

These issues have created a seemingly insurmountable challenge for healthcare professionals: (1) the amount of data provided in an echocardiogram is extensive and can be overwhelming; (2) healthcare professionals are limited on the amount of time they can spend evaluating each echocardiogram; and (3) current echocardiogram systems do not provide an efficient presentation of information, and (4) current echocardiogram systems do not link historical information and results. These problems, among others, cause tremendous strain on healthcare professionals in their battle against cardiovascular disease and can lead to inaccurate diagnosis.

This disclosure relates to interpreting echocardiograms. More specifically, this disclosure provides a cohesive production of data so that a healthcare professional can more quickly, more efficiently, and more accurately diagnose and or make decisions regarding a patient's health.

Thus it is an object of the disclosure to provide a system and method of interpreting an echocardiogram that provides a healthcare professional with clean, organized and easy to understand information which leads to more effective patient care.

Efficiency Objectives

Another object of the disclosure is to provide a system and method of interpreting an echocardiogram that is an efficient and user friendly diagnosing tool.

Yet another object of the disclosure is to provide a system and method of interpreting an echocardiogram that provides information in a quickly understandable format.

Yet another object of the disclosure is to provide a system and method of interpreting an echocardiogram that presents a digestible amount of the correct information needed for specific evaluations.

Another object of the disclosure is to provide a system and method of interpreting an echocardiogram that provides highlighted fields of a suggested interpretation and/or diagnosis.

Yet another object of the disclosure is to provide a system and method of interpreting an echocardiogram that alerts a healthcare professional to varying data from previous tests.

Another object of the disclosure is to provide a system and method of interpreting an echocardiogram that provides critical finding alerts.

Yet another object of the disclosure is to provide a system and method of interpreting an echocardiogram that precludes careless and/or mistaken data entry.

Another object of the disclosure is to provide a system and method of interpreting an echocardiogram that highlights discordant data.

Yet another object of the disclosure is to provide a system and method of interpreting an echocardiogram that prompts a healthcare professional to note a reason for discordant data.

Another object of the disclosure is to provide a system and method of interpreting an echocardiogram that provides the ability for a healthcare professional to easily change or amend data provided by an echocardiogram.

Yet another object of the disclosure is to provide a system and method of interpreting an echocardiogram that creates digestible reports.

Another object of the disclosure is to provide a system and method of interpreting an echocardiogram that creates comparison with previous data and comparison with previous data interpretation.

Yet another object of the disclosure is to provide a system and method of interpreting an echocardiogram that provides a means for sharing information with others.

Patient Care Objectives

Another object of the disclosure is to provide a system and method of interpreting an echocardiogram that helps the healthcare professional effectively treat patients.

Yet another object of the disclosure is to provide a system and method of interpreting an echocardiogram that produces more-consistent and reproducible results.

Another object of the disclosure is to provide a system and method of interpreting an echocardiogram that provides advice to a healthcare professional on selection of parameters related to the structure or function of a patient's heart.

Historical Analysis Objectives Another object of the disclosure is to provide a system and method of interpreting an echocardiogram that provides a visual means for viewing a patient's history.

Yet another object of the disclosure is to provide a system and method of interpreting an echocardiogram that incorporates in-line information related to a patient's history.

Another object of the disclosure is to provide a system and method of interpreting an echocardiogram that provides an echocardiogram interpretation collated with historical information.

Yet another object of the disclosure is to provide a system and method of interpreting an echocardiogram that incorporates historical information with current information to assist diagnosis.

Another object of the disclosure is to provide a system and method of interpreting an echocardiogram that provides a healthcare professional with a visual tool to the patient's evaluation.

Objective Based Evaluation Objectives

Another object of the disclosure is to provide a system and method of interpreting an echocardiogram that provides an evidence-based management system for healthcare professionals.

Yet another object of the disclosure is to provide a system and method of interpreting an echocardiogram that provides a means for healthcare professionals to produce objective based interpretations.

Accreditation Objectives

Another object of the disclosure is to provide a system and method of interpreting an echocardiogram that provides a means for healthcare professionals to ensure they are meeting Intersocietal Accreditation Commission (IAC) guidelines.

Yet another object of the disclosure is to provide a system and method of interpreting an echocardiogram that provide more consistent interpretations of echocardiograms.

These and other objects, features, or advantages of the present disclosure will become apparent from the specification and claims.

### SUMMARY OF THE DISCLOSURE

A system and method for interpreting an echocardiogram is presented which includes receiving a current data set as well as one or more prior data sets having a plurality of values from the performance of an echocardiogram. Storing the current data sets in memory of a computing device and placing the current data set into a plurality of fields using on an interactive user page using a processor and software and comparing the values of the current data set with values of the prior data set and indicating when current values vary more than a predetermined amount from the prior data set. The system generates recommended results based on evaluation of the values using predetermined guidelines. The system indicates to the cardiologist when the cardiologist makes a determination that is discordant with an expected determination based on the values and applicable guidelines. The system also displays prior determinations along with present determination options as well as provides a hover function that provides a popup box with all prior determinations or values from prior echocardiograms. The system also separates the review of the echocardiogram into a plurality of tabs, where each tab is directed to a subset of the review process and includes a progress bar that shows the amount of completion. The system also prevents the cardiologist from submitting a review that is incomplete according to applicable standards. The system automatically generates summary comments and detailed comments based on the determinations of the cardiologist. The system automatically generates a schematic representation of the heart of the patient including symbols and numerical values based upon the cardiologist's determinations. The system also strikes out discordant values from reports when they are in conflict with a cardiologist's determination. The system generates a critical finding prompt that requires the cardiologist to contact the treating physician when a critical finding is made. The system also automatically generates a unique report that includes an overall impression, a schematic representation, a wall motion diagram, detailed findings and a data sheet.

### BRIEF DESCRIPTION OF THE DRAWINGS

FIG. 1 is a plan view of a demonstrative overview of the system and method for interpreting an echocardiogram; the view showing the patient, treating physician, echo tech, the echocardiogram testing device, the data set on one side, and the cardiologist on the other side, which are connected by an electronic network between computing devices;

FIG. 2 is another plan view of a demonstrative overview of the system and method for interpreting an echocardiogram;

FIG. 3 is another plan view of a demonstrative overview of the system and method for interpreting an echocardiogram;

FIG. 4 is a plan view of an interactive user display showing a patient search page of the system and method for interpreting an echocardiogram;

FIG. 5 is a plan view of an interactive user page associated with a demographics tab of the interactive user display of the system and method for interpreting an echocardiogram;

FIG. 6 is a plan view of an interactive user page associated with a data sheet tab of the interactive user display of the system and method for interpreting an echocardiogram;

FIG. 7 is a plan view of an interactive user page associated with a left ventricle tab of the interactive user display of the system and method for interpreting an echocardiogram;

FIG. 8 is a plan view of an interactive user page associated with a left atrium tab of the interactive user display of the system and method for interpreting an echocardiogram;

FIG. 9 is a plan view of an interactive user page associated with a right ventricle tab of the interactive user display of the system and method for interpreting an echocardiogram; the view showing the selection of the “Severe+” descriptor under the “Right Ventricle Size” evaluation section (by the placement of the dark circle, or selection indicator, within the input area); and the view showing the selection of the “Mild-” descriptor under the “Right Ventricle Function” evaluation section (by the placement of the dark circle, or selection indicator, within the input area).

FIG. 10 is a plan view of an interactive user page associated with a right atrium tab of the interactive user display of the system and method for interpreting an echocardiogram; the view showing the selection of the “Mod+” descriptor under the “Right Atrial Size” evaluation section (by the placement of the dark circle, or selection indicator, within the input area); and the view showing the selection of the “Normal” descriptor under the “Right Atrial Pressure” evaluation section (by the placement of the dark circle, or selection indicator, within the input area) this is when the suggested finding indicator is placed around the “Inc+” selection option.

FIG. 11 is a plan view of an interactive user page associated with an aortic valve tab of the interactive user display of the system and method for interpreting an echocardiogram; the view showing the selection of the “Mild” descriptor under the “Stenosis” evaluation section (by the placement of the dark circle, or selection indicator, within the input area).

FIG. 12 is a plan view of an interactive user page associated with a mitral valve tab of the interactive user display of the system and method for interpreting an echocardiogram;

FIG. 13 is a plan view of an interactive user page associated with a tricuspid valve tab of the interactive user display of the system and method for interpreting an echocardiogram; the view showing the selection of the “Bioprosthesis” descriptor under the “Repair/Prosthesis” evaluation section (by the placement of the dark circle, or selection indicator, within the input area); the view showing the selection of the “TR” descriptor with a check mark within the evaluation section and further selection of the “Tr” descriptor selection option under the “Overall Regurgitation” evaluation section (by the placement of the dark circle, or selection indicator, within the input area); the view showing the selection of the “Mod” descriptor under the “Stenosis” evaluation section (by the placement of the dark circle, or selection indicator, within the input area); the view showing the selection of the “Mild” descriptor under the “PASP/PHTN” evaluation section (by the placement of the dark circle, or selection indicator, within the input area) this is when the suggested finding indicator is placed around the “NWV” (Not Well Visualized) selection option.

FIG. 14 is a plan view of an interactive user page associated with a pulmonic valve tab of the interactive user display of the system and method for interpreting an echocardiogram; the view showing the selection of the “Thickened” descriptor under the “Leaflet Morphology” evaluation section (by the placement of the dark circle, or selection indicator, within the input area); the view showing the selection of the “Bioprosthesis” descriptor under the “Prosthesis” evaluation section (by the placement of the dark circle, or selection indicator, within the input area), with further selections of “Melody Valve” descriptor under the under the “Type” evaluation section, and “Normal Thickness” under the “Appearance” evaluation section (by the placement of the dark circle, or selection indicator, within the input area); the view showing the selection of the “1+” descriptor under the “Regurgitation” evaluation section (by the placement of the dark circle, or selection indicator, within the input area); the view showing the selection of the “Mod” descriptor under the “Stenosis” evaluation section (by the placement of the dark circle, or selection indicator, within the input area);

FIG. 15 is a plan view of an interactive user page associated with an aorta tab of the interactive user display of the system and method for interpreting an echocardiogram;

FIG. 16 is a plan view of an interactive user page associated with a pericardium tab of the interactive user display of the system and method for interpreting an echocardiogram;

FIG. 17 is a plan view of an interactive user page associated with an interatrial septum tab of the interactive user display of the system and method for interpreting an echocardiogram;

FIG. 18 is a plan view of an interactive user page associated with a pulmonary artery tab of the interactive user display of the system and method for interpreting an echocardiogram;

FIG. 19 is a plan view of an interactive user page associated with an extracardiac tab of the interactive user display of the system and method for interpreting an echocardiogram;

FIG. 20 is a plan view of an interactive user page associated with a mechanical circulatory support tab of the interactive user display of the system and method for interpreting an echocardiogram; the view showing the selection of the “LVAD” descriptor with a check mark within the evaluation section under the “Mechanical Support Devices” evaluation section; the view showing the selection of the “Normal” descriptor selection option under the “Left Ventricle Function” evaluation section (by the placement of the dark circle, or selection indicator, within the input area) and further selection of “57” under the “EF” percentage; the view showing the selection of the “Mild-” descriptor selection option under the “Left Ventricle Function” evaluation section (by the placement of the dark circle, or selection indicator, within the input area);

FIG. 21 is a plan view of an interactive user page associated with a post-procedure tab of the interactive user display of the system and method for interpreting an echocardiogram;

FIG. 22 is a plan view of an interactive user page associated with a Finalize menu tab of the interactive user display of the system and method for interpreting an echocardiogram;

FIG. 23 is a plan view of a the first page of a report generated after completion of the interactive user pages of the interactive user display of the system and method for interpreting an echocardiogram; the view showing the overall impression of the report;

FIG. 24 is a plan view of a the second page of a report generated after completion of the interactive user pages of the interactive user display of the system and method for interpreting an echocardiogram; the view showing the schematic representation and wall motion diagram of the report;

FIG. 25 is a plan view of a the third page of a report generated after completion of the interactive user pages of the interactive user display of the system and method for interpreting an echocardiogram; the view showing the detailed findings of the report;

FIGS. 26 and 27 is a plan view of a the third page of a report generated after completion of the interactive user pages of the interactive user display of the system and method for interpreting an echocardiogram; the view showing the data sheet of the report;

FIG. 28 is a plan view of an example of a schematic representation of a report generated by the system and method for interpreting an echocardiogram; the view showing severe tricuspid regurgitation (4+) and critical aortic stenosis;

FIG. 29 is a plan view of an example of a schematic representation of a report generated by the system and method for interpreting an echocardiogram; the view showing mild tricuspid regurgitation (1+) and mild aortic stenosis;

FIG. 30 is a plan view of an example of a schematic representation of a report generated by the system and method for interpreting an echocardiogram; the view showing mild to moderate tricuspid regurgitation (2+) and moderate aortic stenosis;

FIG. 31 is a plan view of an example of a schematic representation of a report generated by the system and method for interpreting an echocardiogram; the view showing moderate tricuspid regurgitation (3+) and severe aortic stenosis;

FIG. 32 is a plan view of a portion of an interactive user page associated with an aortic valve tab of the interactive user display of the system and method for interpreting an echocardiogram; the view showing a suggested finding indicator is placed around the “Mild” descriptor under the “Stenosis” descriptor;

FIG. 33 is a plan view of a portion of an interactive user page associated with an aortic valve tab of the interactive user display of the system and method for interpreting an echocardiogram; the view showing a suggested finding indicator is placed around the “Mild” descriptor under the “Stenosis” descriptor and the view showing the selection of the “Mild” descriptor (by the placement of the dark circle, or selection indicator, within the input area), resulting in a concordant determination;

FIG. 34 is a plan view of a portion of a report generated after completion of the interactive user pages of the interactive user display of the system and method for interpreting an echocardiogram according to that shown in FIGS. 33 and 34; the view showing the inclusion of the “AVA (CE) 1.7 sq. cm.” in the overall impression indicating a concordant determination;

FIG. 35 is a plan view of a portion of a report generated after completion of the interactive user pages of the interactive user display of the system and method for interpreting an echocardiogram according to that shown in FIGS. 33 and 34; the view showing the inclusion of the “AVA (CE) 1.7 cm²” in the data sheet indicating a concordant determination;

FIG. 36 is a plan view of a portion of an interactive user page associated with an aortic valve tab of the interactive user display of the system and method for interpreting an echocardiogram; the view showing a suggested finding indicator is placed around the “Mild” descriptor under the “Stenosis” descriptor;

FIG. 37 is a plan view of a portion of an interactive user page associated with an aortic valve tab of the interactive user display of the system and method for interpreting an echocardiogram; the view showing a suggested finding indicator is placed around the “Severe” descriptor under the “Stenosis” descriptor.

FIG. 37A is a plan view of a portion of an interactive user page associated with an aortic valve tab of the interactive user display of the system and method for interpreting an echocardiogram; the view showing a suggested finding indicator is placed around the “Severe” descriptor under the “Stenosis” descriptor; and the view showing the selection of the “Severe” descriptor (by the placement of the dark circle, or selection indicator, within the input area).

FIG. 38 is a plan view of a portion of a report generated after completion of the interactive user pages of the interactive user display of the system and method for interpreting an echocardiogram according to that shown in FIG. 37 and FIG. 37A; the view showing the inclusion of the “AVA (CE) 0.8 sq. cm.” in the overall impression indicating a concordant determination;

FIG. 39 is a plan view of a portion of an interactive user page associated with an aortic valve tab of the interactive user display of the system and method for interpreting an echocardiogram; the view showing a suggested finding indicator is placed around the “Severe” descriptor under the “Stenosis” descriptor and the view showing the selection of the “Mild” descriptor, resulting in a discordant determination;

FIG. 40 is a plan view of FIG. 39 showing the discordant selection indicator which alerts the physician to the presence of discordant data;

FIG. 41 is a plan view of a portion of a report generated after completion of the interactive user pages of the interactive user display of the system and method for interpreting an echocardiogram according to that shown in FIGS. 39 and 40; the view showing the omission of the “AVA (CE) 0.8 sq. cm.” in the overall impression as it was discordant with the determination of the cardiologist;

FIG. 42 is a plan view of a portion of a report generated after completion of the interactive user pages of the interactive user display of the system and method for interpreting an echocardiogram according to that shown in FIGS. 39, 40 and 41; the view showing the “AVA (CE) cm²” in the data sheet in strikethrough text indicating a discordant determination;

FIG. 43 is a plan view of a portion of an interactive user page associated with an left ventricle tab of the interactive user display of the system and method for interpreting an echocardiogram; the view showing a hover function popup box listing the prior determinations by date;

FIG. 44 is a plan view of a portion of an interactive user page associated with left ventricle tab of the interactive user display of the system and method for interpreting an echocardiogram; the view showing a concordant determination for the left ventricle cavity size;

FIG. 45 is a plan view of a portion of a report generated after completion of the interactive user pages of the interactive user display of the system and method for interpreting an echocardiogram according to that shown in FIG. 44; the view showing the “LVIDd: 50 mm” in the data sheet in normal text indicating a concordant determination;

FIG. 46 is a plan view of a portion of an interactive user page associated with left ventricle tab of the interactive user display of the system and method for interpreting an echocardiogram; the view showing a discordant determination for the left ventricle cavity size;

FIG. 47 is a plan view of a portion of a report generated after completion of the interactive user pages of the interactive user display of the system and method for interpreting an echocardiogram according to that shown in FIG. 46; the view showing the “LVIDd: *50 mm” in the data sheet in with the asterisk indicating a discordant determination;

FIG. 48 is a of a portion of an interactive user page associated with pericardium tab of interactive user display of the system and method for interpreting an echocardiogram; the view showing a pop-up box indicating to the cardiologist that the report cannot be finalized because mandatory findings have not yet been made;

FIG. 49 is a of a portion of an interactive user page associated with pericardium tab of interactive user display of the system and method for interpreting an echocardiogram; the view showing the Finalize button presented in a treatment indicating that it can now be selected after all of the mandatory findings have been made;

FIG. 50 is a of a portion of an interactive user page associated with the interactive user display of the system and method for interpreting an echocardiogram; the view showing a pop-up box indicating to the cardiologist that a critical finding has been made, which includes a critical finding alert indicating to cardiologist that they must call and/or make verbal communication with the treating physician before they can complete the study;

FIG. 51 is a of a portion of an interactive user page associated with the interactive user display of the system and method for interpreting an echocardiogram; the view showing a pop-up box indicating to the cardiologist that a critical finding has been made, which includes a critical finding alert indicating to cardiologist that they must call and/or make verbal communication with the treating physician before they can complete the study; the view showing the cardiologist clicked that they verbally communicated the critical finding to the treating physician;

FIG. 52 is a of a portion of an interactive user page associated with left atrium tab of interactive user display of the system and method for interpreting an echocardiogram; the view showing a box evaluation section directed to “LAA Occlusion Device”;

FIG. 53 is a of a portion of an interactive user page associated with left atrium tab of interactive user display of the system and method for interpreting an echocardiogram; the view showing a box evaluation section directed to “LAA Occlusion Device”; the view showing a Watchman device has been selected under the input area and sub-groups of further selections are presented that specifically relate to the Watchman device.

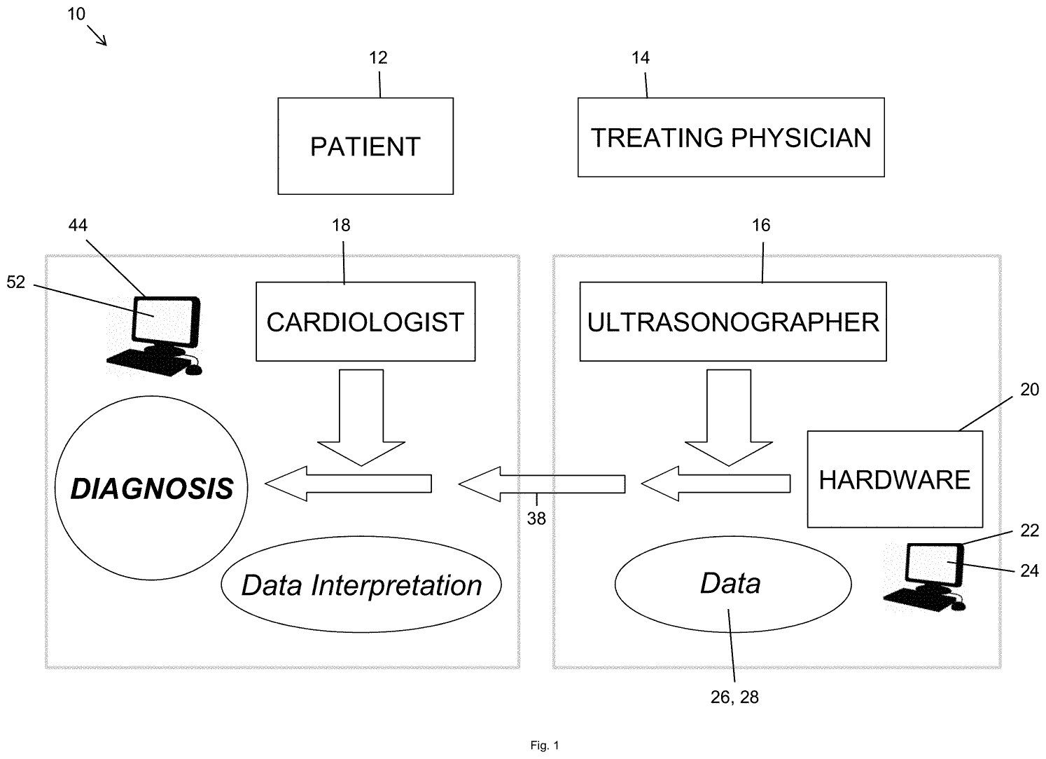

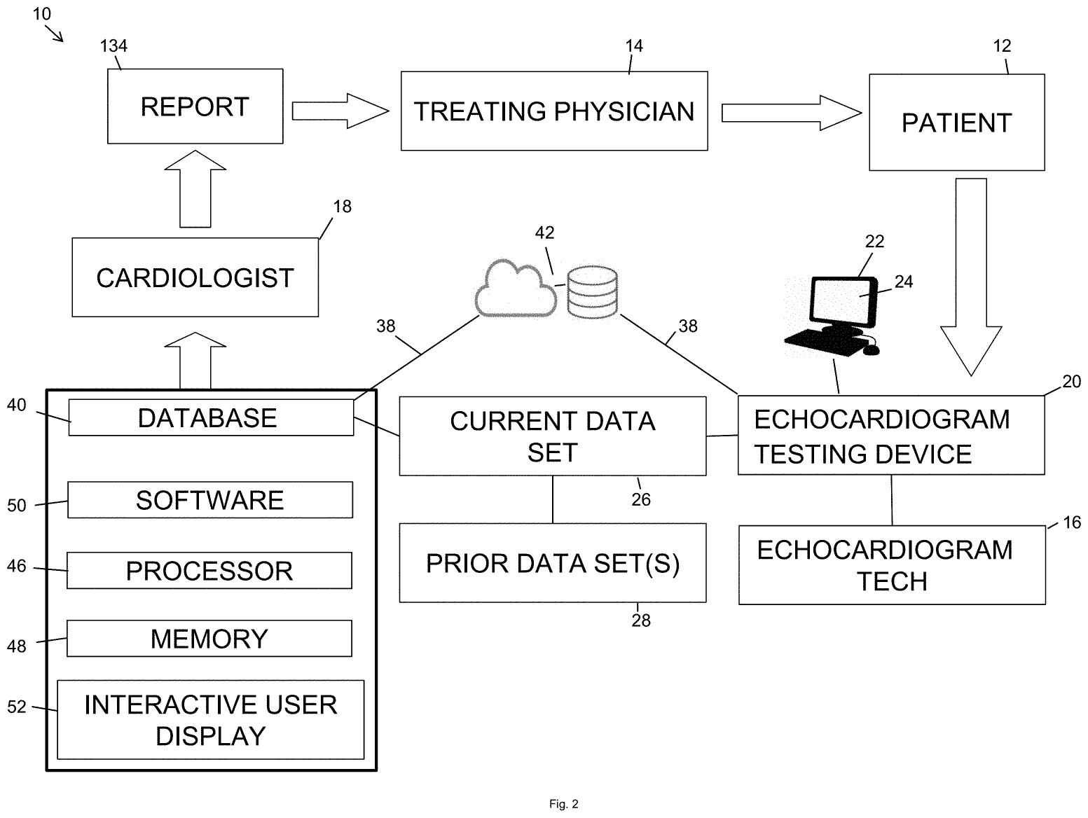

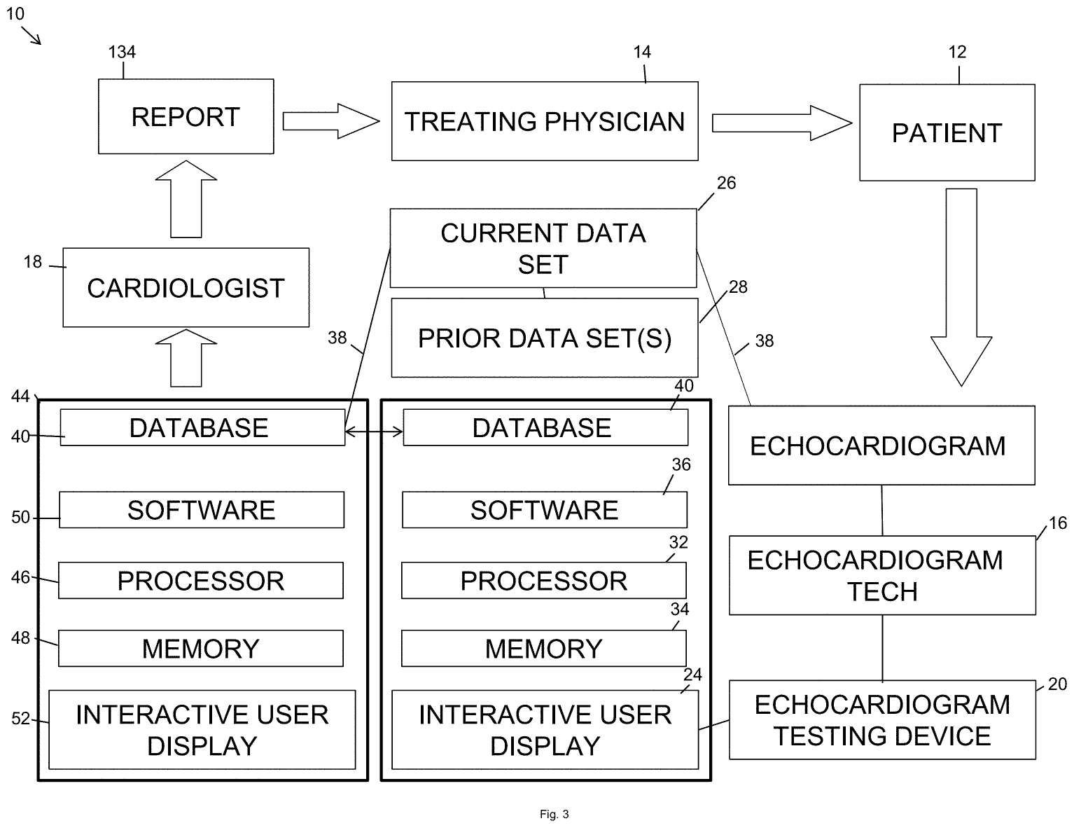

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the present disclosures. The following detailed description is, therefore, not to be taken in a limiting sense, and the scope of the present disclosure is defined only by the appended claims, along with the full scope of equivalents to which such claims are entitled.

As used herein, the terminology such as vertical, horizontal, top, bottom, front, back, end and sides are referenced according to the views presented. It should be understood, however, that the terms are used only for purposes of description, and are not intended to be used as limitations. Accordingly, orientation of an object or a combination of objects may change without departing from the scope of the disclosure.

System:

With reference to the figures, a system and method of interpreting an echocardiogram 10 (or simply, system 10) is presented. System 10 is directed toward a computer based system having a interactive user display used in processing and interpreting an echocardiogram. Using the system 10 disclosed herein, cardiologists will be better able to fully and completely evaluate new and existing patients, monitor the progress of patients, and prescribe, diagnose or alter their present treatments. By assisting healthcare professionals involved in echocardiogram performance, evaluation, interpretation, and ultimately treatment, system 10 helps patients by delivering more efficient, accurate, timely, and organized diagnosis. System 10 provides a fully standardized, organized, and value-added evaluation of a patient, particularly with respect to the patient's past, current, or developing cardiovascular issues.

System 10 is formed of any suitable configuration, computer processor device, user interface and program and is configured to facilitate the quick, easy, safe, efficient, and accurate processing and/or diagnosing of information interpreted and presented by the performance of an echocardiogram. In the arrangement shown, as one exemplary embodiment, system 10 includes a patient 12, a treating physician 14, an echocardiogram tech 16 (or echo tech 16), and a cardiologist 18, among others.

The term patient 12 is used herein to describe the person or persons upon which the echocardiogram is performed. Patient 12 may be any person, or animal. The term patient 12 is not meant to be limiting.

The term treating physician 14 is used herein to describe the person or persons that generally are most-involved with treating patient 12 who tend to care for the overall health of the patient 12. Often, the treating physician 14 is a generalist who orders the patient 14 to have an echocardiogram performed on them by an echo tech 16. Often, the results of the echocardiogram are interpreted by a cardiologist 18, who specializes in treatment and diagnosis of heart conditions. The interpretation of the echocardiogram by the cardiologist 18 is then provided to the treating physician 14. With the information from the interpretation of the echocardiogram, the treating physician 14 determines further care for the patient 12. The term treating physician 14 is not meant to be limiting.

The term echocardiogram tech 16 is used herein to describe the person or persons who perform the echocardiogram on the patient 12. The term echo tech and ultrasonographer may also be used interchangeably with echocardiogram tech 16. The echocardiogram tech 16 may be a licensed echocardiogram technician, a nurse, a doctor, a cardiologist, a physician's assistant, or any other person that is capable of performing an echocardiogram on patient 12. The term echocardiogram tech 16 is not meant to be limiting.

The term cardiologist 18 is used herein to describe the person or persons who receive the results of an echocardiogram performed on the patient 12 and interprets them. The cardiologist 18 may be a licensed cardiologist, a nurse, a doctor, a physician's assistant, or any other person that is capable of interpreting the results of an echocardiogram performed on patient 12. The term cardiologist 18 is not meant to be limiting.

Performance of an Echocardiogram:

In the arrangement shown, as one example, an echocardiogram is performed on patient 12 by echo tech 16 using echocardiogram testing device 20 having a computing device 22 with an interactive user display 24. During the performance of the echocardiogram on patient 12 a current data set 26 is generated which includes the data and information from the performance of the current echocardiogram. This current data set 26 is similar to prior data set(s) 28, which includes the data and information from the performance of past echocardiograms. In the arrangement shown, as one example, computing device 22 includes interactive user display 24, processor 32, memory 34, and software 36. These components of computing device 22 work in cooperation with one another to gather and manipulate the information of current data set 26.

In one arrangement, current data set 26, as well as prior data set(s) 28 include a plurality of values, which may be measurements and/or calculations. Current data set 26, as well as prior data set(s) 28, may also include non-numerical data such as demographic information such as name, address, observations, notes and the like. Current data set 26, as well as prior data set(s) 28 may also include any other form of information or data.

In one arrangement, as one example of an echocardiogram, a treating physician 14 suggests their patient 12 undergo an echocardiogram which is often performed by an echocardiogram tech 16. The echocardiogram tech 16 performs an echocardiogram on a patient 12. The echocardiogram tech 16 uses echocardiogram testing device 20 to gather data. Such data relates to, but is not limited to, structural and functional elements of the heart and surrounding cardiovascular system. The echocardiogram tech 16 performs an initial review of the current data set 26. Once the current data set 26 meets the approval of the echocardiogram tech 16, the echo tech 16 uploads or transmits the current data set 26 over electronic network 38 to other components of system 10 for interpretation and diagnosis by cardiologist 18, as is further described herein.

In one arrangement, current data set 28 is transmitted over electronic network 38, which may be an intranet, the internet or any other electronic connection and communication system between computers and/or computer networks to database 40 and/or server or cloud 42. Database 40 is any electronic data storage system, electronic data retrieval system and data organization system. Server or cloud 42 may be any arrangement of data storage devices or servers which may be hosted on location, such as an on-location server, or off site in one or more locations, which is often referred to as the cloud. Electronic network 38, database 40 and server or cloud 42 are used in cooperation with one another to transmit the current data set 26, as well as prior data set(s) 28 (collectively referred to herein as data set 26, 28) from the echo tech 16 to the cardiologist 18 for interpretation by cardiologist 18.

Interpretation of an Echocardiogram:

In the arrangement shown, as one example, once the echocardiogram is performed by echo tech 16 on patient 12 which generates current data set 26, the current data set 26 is transmitted over electronic network 38 to database 40 where it is accessed, reviewed, and interpreted by cardiologist 18.

Computing Device:

In one arrangement, as is shown, system 10 includes a computing device 44 connected with electronic network 38 and/or database 40 and/or server or cloud 42 and includes a processor 46, memory 48, software 50 and an interactive user display 52 among other components. Computing device 44 may be formed of any computing device capable of displaying and manipulating data set 26, 28 in the manners described herein. Computing device 44 may include for example a desktop computer, a laptop computer, a tablet, smart phone, or any other computing device or other interactive device.

Computing device 44 may be a single consolidated component, or alternatively, computing device 44 may be formed of a plurality of interconnected components that may be co-located or located at different geographic locations. Computing device 44 may be cloud based or it may be a hardware based. In addition, the connected components of computing device 44, including processor 46, memory 48, software 50 and interactive user display 52, may be co-located with computing device 44 or located at different geographic locations. That is, computing device 44 may be made of any form of a device or system that individually or collectively performs the computing operations of the system 10.

Processor:

In the arrangement shown, as on example, system 10 includes a processor 46. Processor 46 is formed of any suitable size, shape and design and is configured to facilitate processing of information (including data set 26, 28) in association with computing device 44, memory 48, software 50 and interactive user display 52. Processor 46 may be a single component, such as a microprocessor chip, or alternatively processor 46 may be formed of a plurality of processing components that are connected to one another that may be co-located or located at different geographic locations. Processor 46 sends and receives information and instructions from and as well as to database 40, server or cloud 42, memory 48, software 50 and interactive user display 52. In one arrangement, processor 46 receives information stored in memory 48 and processes this information pursuant to instructions or software 50.

Memory:

In the arrangement shown, as on example, system 10 includes a memory 48. Memory 48 may be formed of any suitable size, shape and design and is configured to facilitate selective storage and retrieval of data (including data set 26, 28) in association with computing device 44, processor 46, software 50 and interactive user display 52. Memory 48 may be a single component, such as a single chip or drive or other memory device, or alternatively memory 48 may be formed of a plurality of memory or storage components that are connected to one another that may be co-located or located at different geographic locations.

Software:

In the arrangement shown, as on example, system 10 includes software 50. Software 50 may be formed of any suitable size, shape and design and is configured to provide instructions, tools, rules and guidelines as to how to operate system 10 and manipulate data set 26, 28. Software 50 may include code, firmware, software and other components and information. Software 50 may be a single consolidated set of instructions and information, or alternatively software 50 may be formed of a plurality of sets of instructions and information that that may be co-located or located at different locations on different components of the system.

Computing device 44, processor 46, memory 48 and software 50 work in cooperation with one another to receive, manipulate, organize and display data set 26, 28 on interactive user display 52 for review and interpretation by cardiologist 18.

Interactive User Display:

In the arrangement shown, as one example, system 10 includes an interactive user display 52 which is configured to show and display information, including data set 26, 28, for review and interpretation by cardiologist 18. In the arrangement shown, as one example, interactive user display 52 is formed of display screen, such as that of a desktop computer, laptop computer, monitor, tablet, smart phone, smart TV, projector, virtual reality display or any other device or form of a display. In the arrangement shown, as one example, interactive user display 52 comprises a series of interactive user display pages 54, however, the interactive user display 52 may consist of a single page or any other method of displaying information on a display as could be adapted to various size screens, devices, or user preferences. The interactive user display 52 can display various patient and/or medical information which is retrieved and/or requested.

Patient Search Page:

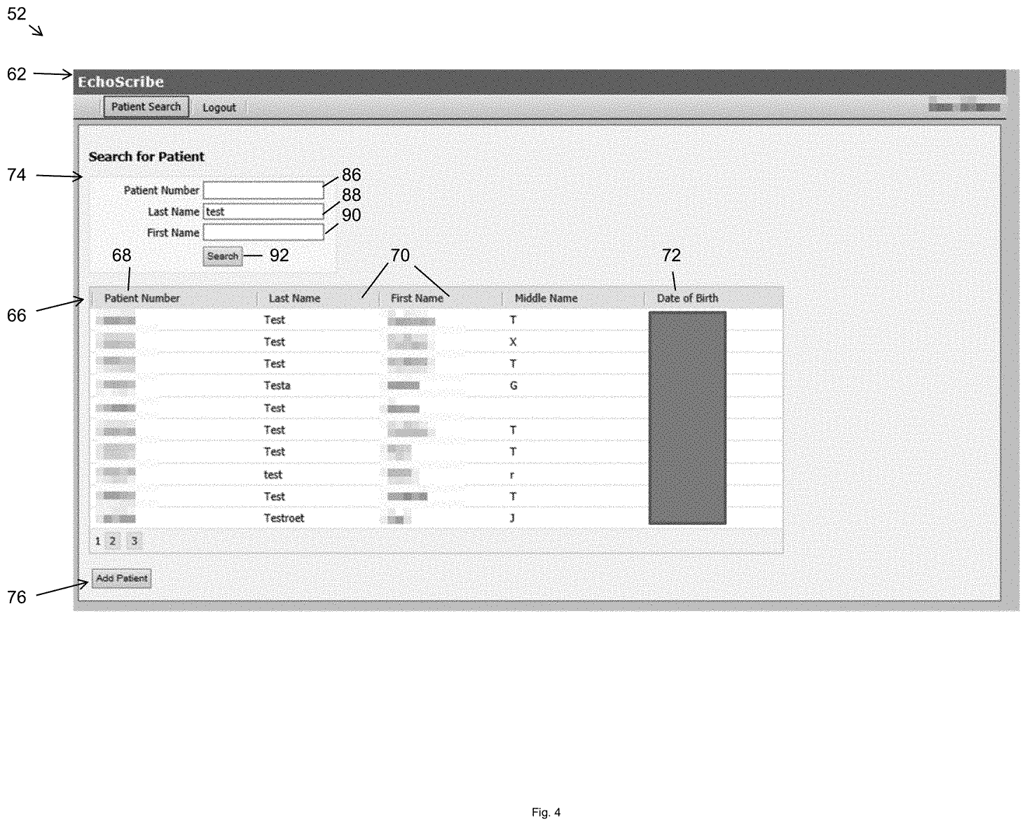

In the arrangement shown, as one example, with reference to FIG. 4, an interactive user display 52 includes a patient search page 62. The patient search page 62 can be arranged in any suitable size, shape, design and manner to display patient information and provide for patient selection search function. In the arrangement shown, as one example, with reference to FIG. 4, patient search page 62 provides a graphical user interface that where patient information is retrieved, displayed and interacted with. In the arrangement shown, as one example, the information is organized and displayed through the graphical user interface as a series of patients 12 in a list which the user can scroll through to make a selection. In the arrangement shown, as one example, the patient search page 62 is the first page encountered after a successful login to the interactive user display 52 of system 10.

In one arrangement, as is shown, the patient search page 62 displays a list of patients 66 including, in the arrangement shown, a patient number 68, a patient name 70, a patient date of birth 72, and any other information as is necessary for patient selection and/or identification. The patient search page 62 can take any form and/or be arranged in a way the user desires for efficient and easy patient selection. In the arrangement shown, patient search page 62 is the first step in recalling the patient information from database 40, server or cloud 42, memory 48 or any other storage device or system for the interpretation of an echocardiogram by cardiologist 18.

Additionally, in the example shown, the patient search page 62 includes a search function 74, that allows for the user to search for a desired patient 12, and an add patient function 76 which allows a user to add a desired patient 12. Once a patient 12 is selected or added, the data set 26, 28 related to that patient 12 is retrieved and displayed in the interactive user display 52.

When a cardiologist 18 selects the patient search function 74, the user will be allowed to search by patient number 68, by patient name 70 (last name, or by first name), or any other search fields desired. In the arrangement shown, as one example, a cardiologist 18 may search by entering a patient number 68 into the patient number field 86, by entering the last name of the patient in the last name field 88, or by entering in the first name of the patient in the first name field 90 and subsequently activating the search button 92. In this option the system 10 will search a particular field in the patient information and retrieve matching or partially matching or related patients. The entire name or patient number need not be entered to populate a list but the more accurate the entry, the more accurate the list generated in the patient search page 62. Once a patient 12 is selected, the cardiologist 18 is taken to the next level of pages 54 of system 10 where the data set 26, 28 related to that patient 12 is retrieved and populated into the appropriate places for a clean and legible representation of the data set 26, 28 in an easily digestible, identifiable, and easy to read manner.

Detailed Pages:

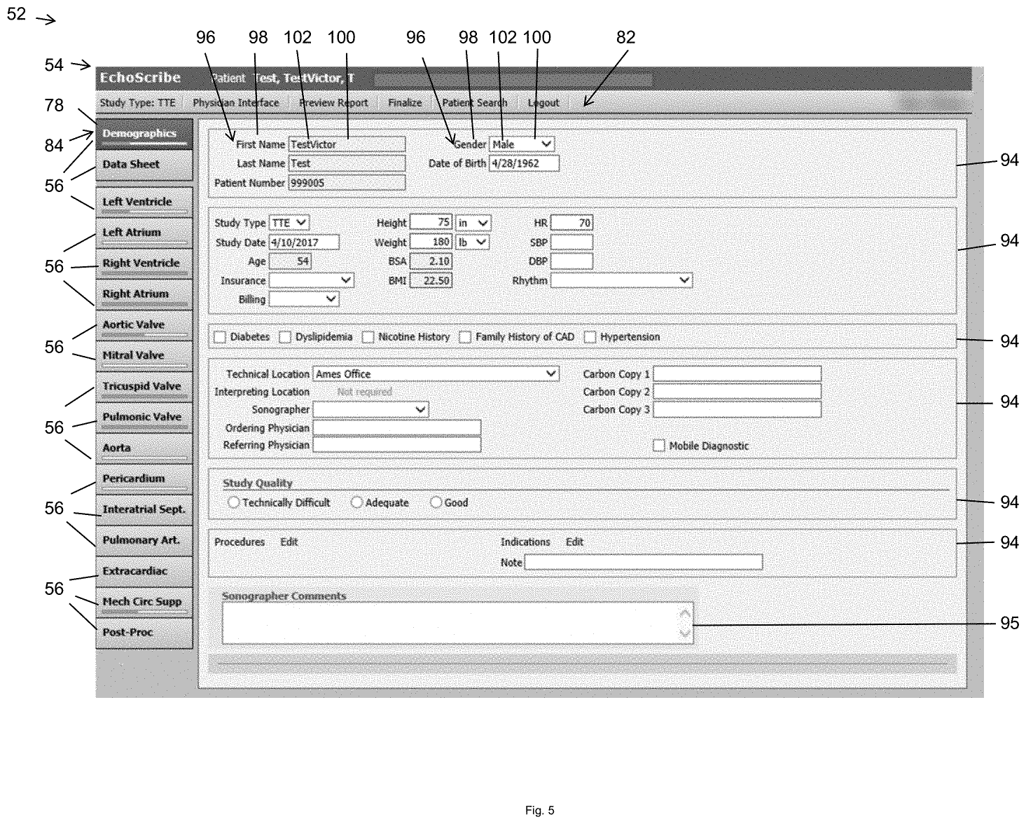

In the arrangement shown, as one example, with reference to FIG. 5 through FIG. 21, once the cardiologist 18 selects a patient 12 from the patient search page 62, the cardiologist 18 is taken to, or has access to, the following interactive user pages 54 of interactive user display 52 that display detailed information regarding patient 12 from the performance of current, as well as prior, echocardiograms. The patient and/or medical information, data set 26, 28, is organized and displayed in the interactive graphical user interface, interactive user display 52, as a series of interactive user pages 54 which are interconnected and/or interlinked by a series of tabs 56. In the arrangement shown, as one example, tabs 56 are arranged vertically along the left side of the interactive user page 54 of interactive user display 52, however any other arrangement is hereby contemplated for use. In the arrangement shown, as one example, the cardiologist 18 or other user may move through the series of pages 54 by navigating this set of tabs 56. In each of these pages cardiologist 18 makes a series of determinations regarding the condition of patient 12 based on the current data set 26 with insight and guidance from prior data set(s) 28, prior findings, and suggestions and assistance from the software and system 10.

In the arrangement shown, as one example, each tab 56 is associated with a different parameter 78. In the arrangement shown, as one example, the various parameters 78 include, but are not limited to: Demographics, Data Sheet, Left Ventricle, Left Atrium, Right Ventricle, Right Atrium, Aortic Valve, Mitral Valve, Tricuspid Valve, Pulmonic Valve, Aorta, Pericardium, Interatrial Septum, Pulmonary Artery, Extracardiac, Mechanical Circulatory Supply, and Post-Procedural. In the arrangement shown, as one example, each parameter 78 identifies a structural element and/or functional element, and/or any other aspect of the condition of patient 12.

In addition to a parameter 78, in the example shown, tabs 56 may include a progress bar 80. Progress bar 80 provides a visual display to the cardiologist 18 which indicates the amount of progress which has been completed for each parameter 78. In one arrangement progress bar 80 reflects the number of required steps that have been performed for each parameter 78 to make the resulting report comply with applicable requirements, guidelines or best practices, such as for example the Intersocietal Accreditation Commission (IAC) guidelines for interpreting an echocardiogram, which may be required for accreditation purposes, insurance purposes or any other purpose. In the arrangement shown, as one example, when a progress bar 80 indicates complete, then the cardiologist 18 has completed all of the applicable requirements and/or steps for review of that parameter 78 or portion of the echocardiogram. In the arrangement shown, as one example, progress bar 80 is an elongated rectangle positioned just below the parameter that begins as a white, or unfilled color, that fills in from left to right as the necessary steps or determinations are made in the related interactive user page 54

As one example, when the Intersocietal Accreditation Commission (IAC) guidelines for interpreting an echocardiogram requires cardiologist 18 to make two determinations for a particular parameter 78, the progress bar 80 shows zero percent complete (the rectangular bar is completely unfilled) before the cardiologist 18 makes any determinations; the progress bar 80 shows fifty percent complete (half way filled in from left to right) after the cardiologist 18 makes one of two required findings or determinations; and the progress bar 80 shows one hundred percent complete (completely filled in) when the cardiologist 18 makes two of two required findings or determinations. In this way, the progress bar 80 associated with each tab 56 and each parameter 78 provides a quick and easy visual indication of the progress of evaluation of the echocardiogram. In one arrangement, the system 10 prevents the completion and submission of a review of a current data set 26 until all progress bars 80 are at one hundred percent complete indicating that the cardiologist 18 has adequately reviewed the current data set 26 and made all the required findings and determinations. This prevents the submission of a review that was not fully completed, or did not comply with the applicable requirements, standards, guidelines or rules. In this way, use of the system 10 improves the quality of the outcome by preventing submission of an incomplete review.

In addition to the tabs 56 appearing on the left side of the interactive user display 52, in the arrangement shown, as one example, the interactive user display 52 includes a set of menu tabs 82 located in a horizontal arrangement across the top of the interactive user display 52. In the arrangement shown, as one example, a set of menu tabs 82 includes, but is not limited to, Study Type, Physician Interface, Preview Report, Finalize, Patient Search, and a Logout as options. When any of these menu tabs 82 are selected, they take the user to the page or location associated with the menu tab 82. The arrangement shown is just one example. Other arrangements are hereby contemplated for use including any other location for the set of menu tabs 82. Other arrangements of tabs 56 and menu tabs 82 are also hereby contemplated for use with the system 10.

Demographics Tab:

In the arrangement shown, as one example, interactive user page 54 of interactive user display 52 includes a demographics tab 84. Demographics tab 84 may be formed of any suitable size, shape, or design and serves to indicate to the user what subset of data set 26, 28 the tab 56 relates to, which in this case is demographic information of patient 12. In one arrangement, as is shown, with reference to FIG. 5, demographics tab 84 is located at the top left corner of the interactive user display 24. In the arrangement shown, as one example, demographics tab 84 is the first interactive user page 54 a cardiologist 18 is taken to after selecting patient 12 through patient search page 62.

In the arrangement shown, as one example, when demographics tab 84 is selected, the interactive user page 54, associated with demographics tab 84 provides an illustration of the details of the patient 12 which includes a subset of information from current data set 26, as well as prior data set(s) 28 when applicable and/or necessary, that relate to the demographic information of patient 12. Organizing, packaging and displaying related demographic information on this interactive user page 54 in a standardized, consolidated and convenient manner aids cardiologist 18 in their difficult task of quickly, accurately and thoroughly reviewing and interpreting this subset of current data set 26. In one arrangement, as is shown, interactive user page 54 associated with demographics tab 84, as well as other tabs 56, displays a series of sections 94 and a comment section 95.

Sections:

In the arrangement shown, as one example, sections 94 contain a further subset of the subset of information from current data set 26, as well as prior data set(s) 28 when applicable and/or necessary, that relate to the demographics of the patient 12. Sections 94 provide further organization to the subset of data set 26 by organizing related information within clearly defined and visually separated segments. In the arrangement shown, sections 94 are outlined by a thin border which provides a visually perceptible organization that the information within the border is to be grouped together as it relates to one another.

Sections 94 are arranged in any suitable size, shape, design or position which aids the cardiologist 18 or other user in their difficult task of quickly, accurately and thoroughly reviewing and interpreting this subset of current data set 26. In the arrangement shown, as one example sections 94 of display page 54 of demographics tab 84 are formed of a series of horizontally elongated boxes, which provide visual separation for related portions of data set 26 which make review of the display page 54 quicker, easier, more accurate and more efficient.

In the arrangement shown, as one example, each demographics section 94 houses a plurality of fields 96 which provide information related to patient 12. In the arrangement shown, as one example, each field 96 is directed to a particular category of information from data set 26, 28. In the arrangement shown, as one example, fields 96 may include a descriptor 98, an input area 100 that receives a value 102 and units 104. Descriptor 98 is any indication or description of what the field 96 is related to. Or, said another way, descriptor 98 describes what the field 96 is in layman's or short-hand form. Input area 100 is a fillable area that receives the related information from data set 26, 28 therein, which is in many cases a value 102. Value 102 is any numerical or non-numerical data, result or value related to field 96. Numerical values 102 may be measurements or calculated values. Non-numerical values 102 may be demographic information, observations, determinations or any other information that is non-numerical in nature. Units 104 describe the value 102 such as weight, length, speed, volume, density, percentage, and as examples may be: inches, pounds, cm, mm, %, or the like. Fields 96 may also employ a plurality of selectable listings that are common. Fields 96 may also drop-down listings. In many fields 96, the system 10 is configured to automatically insert, or auto-populate, the value 102 from data set 26, 28 into input area 100.

As an example, the upper most section 94 of interactive user page 54 of demographics tab 84 includes fields 96 having descriptors 98 including “First Name,” “Last Name,” “Patient Number,” “Gender,” and “Date of Birth,” with an input area positioned next to each descriptor 98 that receives the related value 102 therein.

In the arrangement shown, as one example, a second section 94 is dedicated to information related to the study performed such as type and date of the study. The field 96 for the descriptor 98 “Study Type” includes an input area 100 that employs a dropdown menu including pre-selected common values 102, which are the types of studies commonly performed. The use of field 96 with a dropdown menu having pre-selected common values 102 saves the cardiologist 18 time, reduces human error, creates increased standardization between study reviews, increases accuracy, and makes it easier and faster for the cardiologist 18 to review the echocardiogram. This section 94, as one example, also includes other fields 96 related to patient 12 such as Age, Insurance, Billing, Height, Weight, BSA, BMI, HR, SBP, DBP, and Rhythm. Where possible, the system 10 auto-populates values 102 into the input areas 100 with information from data set 26, 28. These characteristics can provide the cardiologist with useful information about patient 12 that may explain some of the findings of the echocardiogram as well as help with interpretation of the echocardiogram and eventual diagnosis.

In the arrangement shown, as one example, a third section 94 allows the cardiologist 18, or any other user entering information to select relevant conditions that patients 12 often have. In this arrangement, this section 94 includes fields 96 having a selectable listing of a plurality of common conditions including Diabetes, Dyslipidemia, Nicotine History, Family History of CAD, and Hypertension, as one example. Any other condition may be included therein. In this arrangement of fields 96, the descriptor 98 describes the condition (e.g. Diabetes, Dyslipidemia, Nicotine History, Family History of CAD, Hypertension), the input area 100 is a selectable box that is either selected or not selected, and there are no associated units 104.

In the arrangement shown, as one example, a fourth section 94 allows the user to identify the Technical Location related to the performance of the echocardiogram, the Interpreting Location, the Sonographer or echocardiogram tech 16 who performed the echocardiogram, and indicate others which the system can send reports to such as the Ordering Physician, the Referring Physician, and even others through Carbon Copy fields 96 to be notified.

In the arrangement shown, as one example, a fifth section 94 allows the echocardiogram tech 16 or other user to identify the quality of the study which can indicate to the cardiologist 18 the accuracy or precision of the echocardiogram. In this arrangement, this section 94 includes fields 96 having a selectable listing of a plurality of common determinations such as Technically Difficult, Adequate and Good, as one example. Any other descriptor 98 may be included therein. In this arrangement of fields 96, the descriptor 98 describes the quality of the echocardiogram and the input area 100 is a selectable box that is either selected or not selected, and there are no associated units 104. With this information the cardiologist 18 has a lens through which to view the data set 26. This information can help provide the cardiologist 18 with a confidence interval on their determinations and can help them determine whether the need exists to additional tests or echocardiograms.

In the arrangement shown, as one example, a sixth section 94 is provided for entering information related the specific type of echocardiogram that has been performed (e.g. spectral Doppler, which may be pulsed wave (PW) or continuous wave (CW), 3D imaging, 2D imaging, or the like). In this arrangement, cardiologist 18 selects the edit option and selects the specific type or modality of the echocardiogram that was performed on patient 12.

All these sections 94 are shown as an example, other arrangements or fields 96 of information located in the display page 54 associated with demographics tab 84 are hereby contemplated for use.

Where possible, system 10 is configured to auto-populate information from data set 26, 28 into input areas 100 of fields 96 thereby saving the cardiologist 18 the time from doing this as well as eliminating potential human error. The use of fields 96 with a dropdown menu having pre-selected common values 102 saves the cardiologist 18 time, reduces human error, creates increased standardization between study reviews, increases accuracy, and makes it easier and faster for the cardiologist 18 to review the echocardiogram. The use of fields 96 having pre-selected descriptors 98 next to input areas 100 that are a selectable box that is either selected or not selected speeds the evaluation process as well as helps to standardize reviews of echocardiograms between different cardiologists 18.

Comments Section:

In the arrangement shown, as one example, positioned below the plurality of sections 94 a comments section 95 is presented. In this arrangement, comments section 95 allows the echocardiogram tech 16 and/or cardiologist 18 to add or dictate commentary to this portion of display page 54 associated with demographics tab 84. The information added or dictated may then be saved and added to a report which is contemplated herein. In the arrangement shown, comments section 95 includes a free-form fillable field.

In the arrangement shown, as one example, with reference to FIG. 5, this comments section 95 is populated only with comments from the echo tech 16, whereas all other comments sections 95 on other pages are populated with comments from cardiologist 18. Said another way, in one arrangement, this is the only comments section 95 that echo tech 16 has access to. In this arrangement, these comments from echo tech 16 are intended to provide cardiologist 18 with insight regarding the performance of the echocardiogram on patient 12. With this information, cardiologist 18 may have a better insight or understanding of the current data set 26 that allows for a more-accurate interpretation and diagnosis. In one arrangement, this comments section 95 allows for a conversation or commentary to occur between echo tech 16 and cardiologist 18. In one arrangement, these comments from echo tech 16 are used only for the understanding by cardiologist 18 and are not included within the report 134. As one example, echo tech 16 may enter into comments sections 95 “This was a particularly difficult echocardiogram, you may want to consider performing another echocardiogram for more-reliable data.”

Data Sheet Tab:

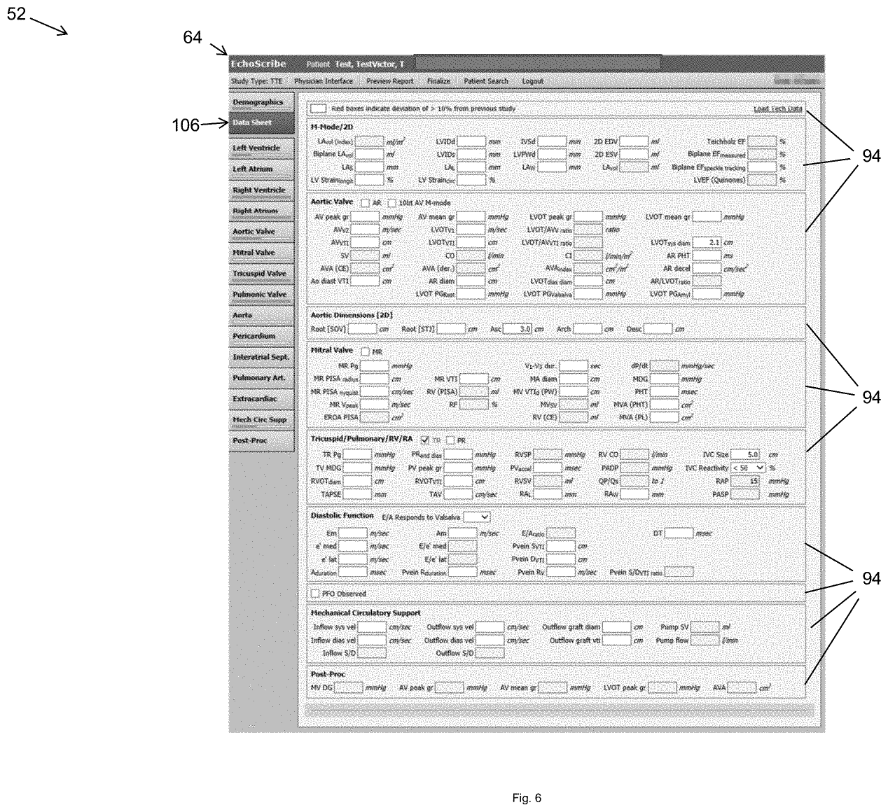

In the arrangement shown, as one example, interactive user display 52 includes a data sheet tab 106. Data sheet tab 106 may be formed of any suitable size, shape, or design and serves to indicate to the user what subset of data set 26, 28 the tab 56 relates to, which in this case is an overall data sheet of information from the performance of an echocardiogram on patient 12. In one arrangement, as is shown, with reference to FIG. 6, data sheet tab 106 is positioned just below demographics tab 84 which is located at the top left corner of the interactive user display 24. In the arrangement shown, as one example, data sheet tab 106 is the second interactive user page 54 a cardiologist 18 is taken to, after completing review of the demographics tab 84. However the cardiologist 18 may freely move among interactive user pages 54 by selecting tabs 56.

In the arrangement shown, as one example, the interactive user page 54, associated with data sheet tab 106 provides an organized overview of some or all of the data collected during the echocardiogram from current data set 26, as well as prior data set(s) 28 when applicable and/or necessary. This information is separated into a plurality of sections 94 of related information. In the arrangement shown, as one example, sections 94 of the interactive user page 54 are elongated rectangular boxes that are stacked vertically along the page. In the arrangement shown, as one example, each section 94 is directed to a different grouping of information from current data set 26. In the arrangement shown, as one example, sections 94 include a plurality of fields 96 having descriptors 98, input areas 100 that receive values 104 and have units 104 associated therewith. Where possible, the system 10 auto-populates values 102 into the input areas 100 with information from data set 26, 28. Organizing, packaging and displaying related information on this interactive user page 54 in a standardized, consolidated and convenient manner aids cardiologist 18 in their difficult task of quickly, accurately and thoroughly reviewing and interpreting the information in current data set 26.

In the arrangement shown, as one example, interactive user page 54, associated with data sheet tab 106 includes sections 94 directed to “M-Mode/2D,” “Aortic Valve,” “Aortic Dimensions [2D],” “Mitral Valve,” “Tricuspid/Pulmonary/RV/RA,” “Diastolic Function,” “PFO Observed,” “Mechanical Circulatory Support,” and “Post-Proc” as examples. These sections 94 include a plurality of fields 96 having descriptors 98, input areas 100 that receive values 102 and include units 104. In one arrangement, system 10 is configured to auto-populate the values 102 from current data set 26 into input areas 100.

Calculated Values:

In the arrangement shown, as one example, some of the fields 96 receive values 102 straight from current data set 26 whereas other fields receive values 102 that are calculated values based on calculations of other values 102 from current data set 26. In the arrangement shown, as one example, the interactive user display 52 of data sheet tab 106 indicates to the cardiologist 18 when a value 102 is calculated from other values 102 by providing a treatment to the field 96 and/or descriptor 98, input area 100, value 102 and/or units 104 that are calculated. In the arrangement shown, as one example, the treatment to calculated values 102 is done by highlighting the area of input area 100 with a grey background fill, whereas the non-calculated values 102 have no highlighting, or no fill, or a white background in the input area 100. While highlighting calculated values 102 is shown as one way of indicating when a value 102 is a calculated value, any other manner of indicating a calculated value is hereby contemplated by providing any other treatment, such as bolding the text, highlighting the text, italicizing the text, underlining the text, using a different font, using a unique color from non-calculated values (such as green, blue or purple when non-calculated values are shown in black), providing a box around the value 102 or field 96, or any other treatment.

In one arrangement, when the user hovers over, clicks or otherwise interacts with a calculated value 102, the formula or algorithm is displayed in a pop-up box and/or the other values 102 that are used in the calculated value 102 are indicated. This indication can be any type of a treatment such as bolding them, highlighting them, italicizing them, underlining them, pointing to them with lines or arrows, making them increase in size, making them flash, pulse or otherwise move or change dimensions, using a different font, using a unique color, providing a box around them, or any other treatment or any combination thereof is hereby contemplated for use.

Providing this information related to calculated values 102 provides a cardiologist 18 with a better understanding of the origin of the calculated values 102 which may provide the cardiologist 18 with better insight into the current data set 26 and the condition of patient 12. That is, by indicating when a value 102 is calculated from other values 102, and by showing the cardiologist 18 how the value 102 is calculated and what other values 102 are used from the current data set 26, when the calculated value 102 is peculiar or out of line with other values 102, the cardiologist 18 can quickly and easily review the underlying values 102 and the calculation to determine whether they provide insight into or help explain the calculated value 102.

Indication when Current Value Varies from Prior Value:

In the arrangement shown, as one example, one of the benefits of the system 10 is that it provides a comparison of values 102 of the current data set 26 to values 102 of prior data set(s) 28. In one arrangement, system 10 indicates when a value 102 of current data set 26 varies from that value 102 of the prior data set 28 by more than a predetermined amount. In the arrangement shown, as one example, as is shown in the first and upper most section 94, system 10 indicates when a value 102 of current data set 26 deviates more than ten percent from the value 102 of the prior data set 28 by placing a red box around the input area 100 of the value 102 of the field 96.

While placing a red box around values 102 is shown as one way of indicating when a value 102 varies greater than a predetermined amount, any other manner of indicating a value 102 varies from prior data set 28 is hereby contemplated by providing any other treatment, such as bolding the text, highlighting the text, italicizing the text, underlining the text, using a different font, using a unique color, providing a box around the value 102 or field 96, causing the value 102 to flash, pulsate or strobe, providing asterisks or other text, or any other treatment.

While a predetermined variance of ten percent is used in the example shown, any other variance is hereby contemplated for use including one, two, three, four, five, six, seven, eight, nine, eleven, twelve, thirteen, fourteen, fifteen, sixteen, seventeen, eighteen, nineteen, twenty, twenty one, twenty two, twenty three, twenty four, twenty five or more percent or any range therein. Alternatively boundaries can be used, such as when a value 102 is above and/or below a certain predetermined value or outside of a certain range. Alternatively formulas or algorithms may be used to calculate when a value 102 varies greater than an acceptable amount based on one or multiple prior data sets 28, such as deviating too much from a mean value or average or moving average or the like.

While in the arrangement shown, only a single level of variance is indicated (greater than ten percent from the prior data set 28) multiple levels can be indicated. As one example, two levels of variance may be indicated by indicating in a first color when a value 102 varies more than five percent but less than ten percent from the prior data set 28 and indicating in a second color when the value 102 varies more than ten percent from the prior data set 28. As another example, three levels of variance may be indicated by indicating in a first treatment when a value 102 varies more than five percent but less than ten percent from the prior data set 28, indicating in a second treatment when the value 102 varies more than ten percent but less than fifteen percent from the prior data set 28, and indicating in a third treatment when the value 102 varies more than fifteen percent from the prior data set 28.

By providing essentially all of the values 102 of the current data set 26 on a single interactive user page 54 and providing a treatment to values 102 in the current data set 26 that vary greater than a normal or predetermined amount from the prior data set 28, cardiologist 18 can quickly and easily get an overview of the condition of the patient 12 and is alerted to whether there has been a substantial change, which may be due to an actual change in the patient 12, or it can be due to the manner in which the current data set 26 was collected and the manner the echocardiogram was performed.

This feature, comparison of values 102 with prior data set 28 provides a cardiologist 18 with a great level of insight into the current data set 26 and provides context to the values 102 that a cardiologist 18 never previously had before. This insight is important to properly interpreting an echocardiogram and can be the difference between making the optimum determination and an improper determination, which can make the difference between providing optimum care for patient 12 or providing insufficient care for patient 12.

Also, by grouping related values 102 in sections 94, this allows the cardiologist 18 to determine whether different portions of the heart of patient 12 are experiencing changes. For example, when multiple values 102 within one section 94 are indicated as excessively varying from the prior data set 28 this indicates that that portion of the heart of patient 12 is varying greatly. Another benefit of the system 10 is that is quickly and easily allows cardiologist 18 to see values 102 from prior data sets 28 through a hover function 108.

Hover Function & Access to Prior Data Sets:

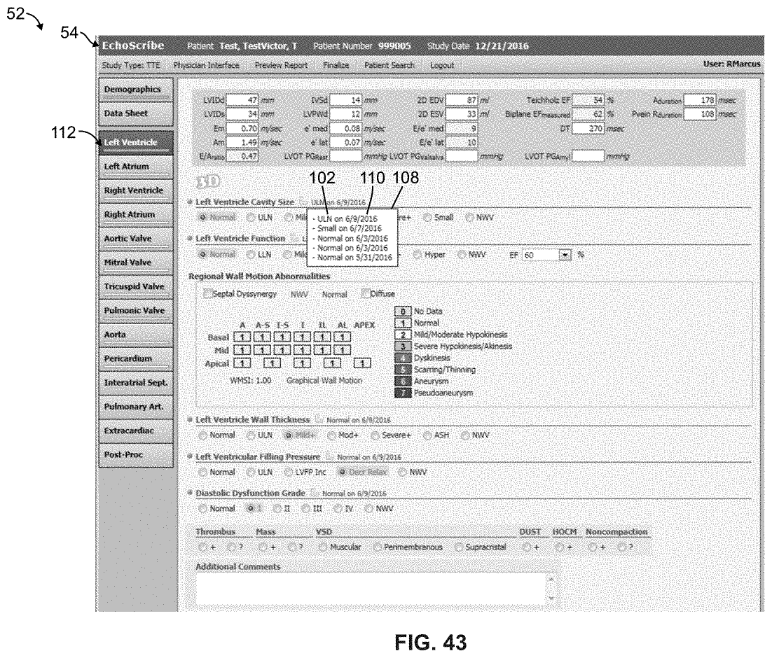

In the arrangement shown, as one example, one of the benefits of the system 10 is that it provides a hover function 108 for some or all of fields 96 and/or values 102 of the current data set 26 that reveals the values 102 and/or findings of prior data set(s) 28 and/or evaluations of prior data sets 28. As is stated herein, to properly interpret a current data set 26 it is important to understand prior data sets 28 from prior echocardiograms. In the arrangement shown, as one example, system 10 includes a hover function 108 that presents values 102 from prior data sets 28 when activated. Hover function 108 is formed of any suitable size, shape and design and is configured to present values 102 from prior data sets 28 when activated. In the arrangement shown, as one example, with reference to FIG. 43, when cardiologist 18 hovers over a field 96 or value 102 for a predetermined amount of time hover function 108 takes the form of a pop-up box is presented that includes a list of the values 102 from prior data sets 28 along with the date 110 of the prior data set 28. In the arrangement shown, as one example, hover function 108 provides a listing of values 102 in chronological order, ordered from most recent at the top to oldest at the bottom, along with the date 110 next to each value 102. This hover function 108 provides a cardiologist 18 with a great level of insight into the current data set 26, by quickly and easily providing historical data for a field 96 and/or value 102, and provides context to the values 102 that a cardiologist 18 never previously had before. This insight is important to properly interpreting an echocardiogram and can be the difference between making the optimum determination and an improper determination, which can make the difference between providing optimum care for patient 12 or providing insufficient care for patient 12.

In one arrangement, the cardiologist 18 can go a step further than just hovering over a field 96 or value 102 to see prior values 102, the cardiologist 18 may click on, select or otherwise interact with field 96 and/or value 102 and be taken to and/or presented with prior data set(s) 28. In doing so, the cardiologist 18, which may or may not have been the cardiologist 18 that evaluated the prior data set(s) 28, is presented with all of the data from prior data set(s) 28, in the same or similar form presented herein. In this way, cardiologist 18 may quickly and easily make themselves aware of the history of the condition of patient 12, which can aid in making the optimum determination in the present data set 26. Due to the time and cost pressures on cardiologists 18, the cardiologist 18 would never have the time to review prior values 102 and/or prior data sets 28 unless they were linked and accessible by hover function 108 in such a quick, easy and efficient manner.

The hover function 108 and access to prior data sets 28 provides a cardiologist 18 with a great level of insight into the current data set 26 and provides context to the values 102 that a cardiologist 18 never previously had before. This insight is important to properly interpreting an echocardiogram and can be the difference between making the optimum determination and an improper determination, which can make the difference between providing optimum care for patient 12 or providing insufficient care for patient 12.

Changing Values:

One of the benefits of the system 10 presented herein is that cardiologist 18 may change some or all of the values 102. The performance of an echocardiogram is an art and interpretation of current data set 26 may require the application and incorporation of clinical acumen into the measured and calculated data. In addition, there are countless factors that may affect the results including differences between patients 12, differences between echo techs 16, differences between echocardiogram testing devices 20, differences between practices, among countless other factors that can cause variations in the results of data set 26, 28.

System 10 allows a cardiologist 18 to omit, override, delete or change a value 102 when they disagree with the value 102. Allowing cardiologist 18 to change and/or delete values 102 eliminates the possibility that errant values 102 make their way into the completed findings or report, which can confuse later reviewers when they see a value 102 in a report that leads to one conclusion but cardiologist 18 comes to a different conclusion. This conflict can bring into question the accuracy and completeness of the current data set 26, the accuracy and performance of the echocardiogram, and the review by cardiologist 18. By enabling a cardiologist 18 to change errant or questionable values 102, this allows the cardiologist 18 to make the values 102 and the determination of the cardiologist 18 congruent with one another, which allows others to rely upon the findings of the echocardiogram with a higher level of confidence. Or, said another way, enabling cardiologist 18 to delete or amend errant or questionable values 102 eliminates internal conflicts which can bring into question the reliability of the echocardiogram and its interpretation by cardiologist 18.

In one arrangement, when cardiologist 18 changes or deletes a value 102, a treatment is applied to the value 102 indicating that the value 102 has been deleted or changed. This treatment may be any manner of indicating a value 102 has been amended such as bolding the text, highlighting the text, italicizing the text, underlining the text, using a different font, using a unique color, providing a box around the value 102 or field 96, providing asterisks providing strikethrough text or any other treatment. In one arrangement, when a value 102 is changed, the prior value 102 may be displayed as well. In another arrangement, when a value 102 is changed the prior value 102 may appear when the field 96 is hovered over, clicked upon or otherwise interacted with by a reviewer. In one arrangement, the name of the cardiologist 18 that changed the value and/or the date of the change is also displayed.

Left Ventricle Tab:

In the arrangement shown, as one example, interactive user display 52 includes a left ventricle tab 112. Left ventricle tab 112 may be formed of any suitable size, shape, or design and serves to indicate to the user what subset of data set 26, 28 the tab 56 relates to, which in this case is information from the performance of an echocardiogram on patient 12 that relates to the left ventricle of the heart of patient 12. In one arrangement, as is shown, left ventricle tab 112 is positioned just below the data sheet tab 106 which is just below the demographics tab 84 which is located at the top left corner of the interactive user display 24. In the arrangement shown, as one example, left ventricle tab 112 is the third interactive user page 54 a cardiologist 18 is taken to, after completing review of the data sheet tab 106. However the cardiologist 18 may freely move among interactive user pages 54 by selecting tabs 56.

Data Section:

In the arrangement shown, as one example, the interactive user page 54, associated with left ventricle tab 112, includes data section 114. In the arrangement shown, as one example, data section 114 is positioned at the top of the interactive user page 54 associated with left ventricle tab 112 and is formed of a subset of the fields 96 having descriptors 98, input areas 100 that include values 102 and have units 104, from the interactive user page 54 of the data sheet tab 106, as is described herein.

In one arrangement, as is shown, data section 114 includes all of the applicable data from current data set 26 that relates to the left ventricle of the heart of patient 12. Data section 114 includes a combination of raw values 102 directly from the current data set 26, as well as calculated values 102. In the arrangement shown, as one example, calculated values 102 are indicated by shaded input areas 100, however any other manner of indicating when a value 102 is calculated is hereby contemplated for use as is described herein. The data section 114 also indicates when a value 102 varies more than a predetermined amount from that value in prior data set 28 as is described herein.

In the arrangement shown, as one example, to provide a visual separation between data section 114 and other sections of the interactive user page 54 of data sheet tab 106, data section 114 is held within a shaded area. This treatment provides a shaded area and border that visually separates the data section 114 from other sections which aids the user when reviewing the contents of the interactive user page 54 associated with left ventricle tab 112.

3D Indicator:

In the arrangement shown, as one example, a 3D indicator 116 is positioned just below the data section 114. 3D indicator 116 is formed of any suitable size, shape and design and is configured to indicate when the echocardiogram performed on patient 12 qualifies as “3D”. In the arrangement shown, as one example, 3D indicator 116 is a selectable icon that when selected indicates the echocardiogram performed on patient 12 qualifies as “3D”. This is important as in some indications if an echocardiogram qualifies as “3D” then the cardiologist 18 can charge more for their review. By placing 3D indicator 116 which is a selectable icon cardiologist 18 may quickly and easily select 3D indicator 116.

Evaluation Sections:

In the arrangement shown, as one example, a plurality of evaluation sections 118 are positioned below 3D indicator 116 and data section 114. Evaluation sections 118 are formed of any suitable size shape and design and are configured to help facilitate cardiologist 18 in their task of quickly, accurately and fully reviewing the data set 26 from the performance of an echocardiogram on patient 12 and evaluating the results of the echocardiogram so that cardiologist 18 may quickly, efficiently and accurately make their determinations.

In the arrangement shown, as one example, evaluation sections 118 include a descriptor 120. Descriptor 120 is any indication or description of what the evaluation section 118 is related to. Or, said another way, descriptor 120 describes what the evaluation section 118 is in layman's or short-hand form or a generally accepted manner.

In the arrangement shown, as one example, positioned adjacent to the descriptor 120 of evaluation section 118 is a prior finding indicator 122. Prior finding indicator 122 indicates to the cardiologist 18 what the finding of that evaluation section 118 was in the prior evaluation and the date 110 of that prior finding. In the arrangement shown, as one example, prior finding indicator 122 also includes hover function 108—which is not unusual in this system 10 as in one arrangement, some if not all of the values, fields, descriptors or other data or findings include a hover function 108 that displays the prior value or finding and/or the partial or complete history of prior values or findings along with the date of those prior values or findings. That is when cardiologist 18 hovers over a descriptor 120, prior finding indicator 122 and/or any other portion of evaluation section 118 for a predetermined amount of time a pop-up box is presented that includes a list of the prior findings from evaluations of prior data sets 28 along with the date 110 of the prior finding. In the arrangement shown, as one example, hover function 108 provides a listing of prior findings in chronological order, ordered from most recent at the top to oldest at the bottom, along with the date 110 next to each prior finding. This hover function 108 provides a cardiologist 18 with a great level of insight into the current data set 26, by quickly and easily providing historical data of prior findings, and provides context to the values 102 of data set 26 that a cardiologist 18 never previously had before. This insight is important to properly interpreting an echocardiogram and can be the difference between making the optimum determination and an improper determination, which can make the difference between providing optimum care for patient 12 or providing insufficient care for patient 12.

In the arrangement shown, as one example, evaluation section 118 includes a selectable listing of a plurality of evaluation options 124. In the arrangement shown, as one example, each evaluation option 124 includes a descriptor 126 along with an input area 128. Descriptor 126 describes the evaluation option 124. Input area 128 is a selectable field that the cardiologist 18 may select to indicate their choice among the plurality of evaluation options 124 which is based on their interpretation of the values 102 in data section 114.

In the arrangement shown, as one example, evaluation options 124 are a list of a plurality of common predetermined determinations for that evaluation section 118. Organizing, packaging and displaying a plurality of predetermined common evaluation options 124 on this interactive user page 54 in a standardized, consolidated and convenient manner aids cardiologist 18 in their difficult task of quickly, accurately and thoroughly reviewing and interpreting this subset of current data set 26. The use of pre-selected determinations evaluation sections 118 saves the cardiologist 18 time, reduces human error, creates increased standardization between study reviews, increases accuracy, and makes it easier and faster for the cardiologist 18 to review the echocardiogram. The use of input areas 128 next to descriptors 126 for evaluation options 124 of evaluation sections 118 allows a cardiologist to quickly select their determination among the portfolio of pre-selected and approved options.

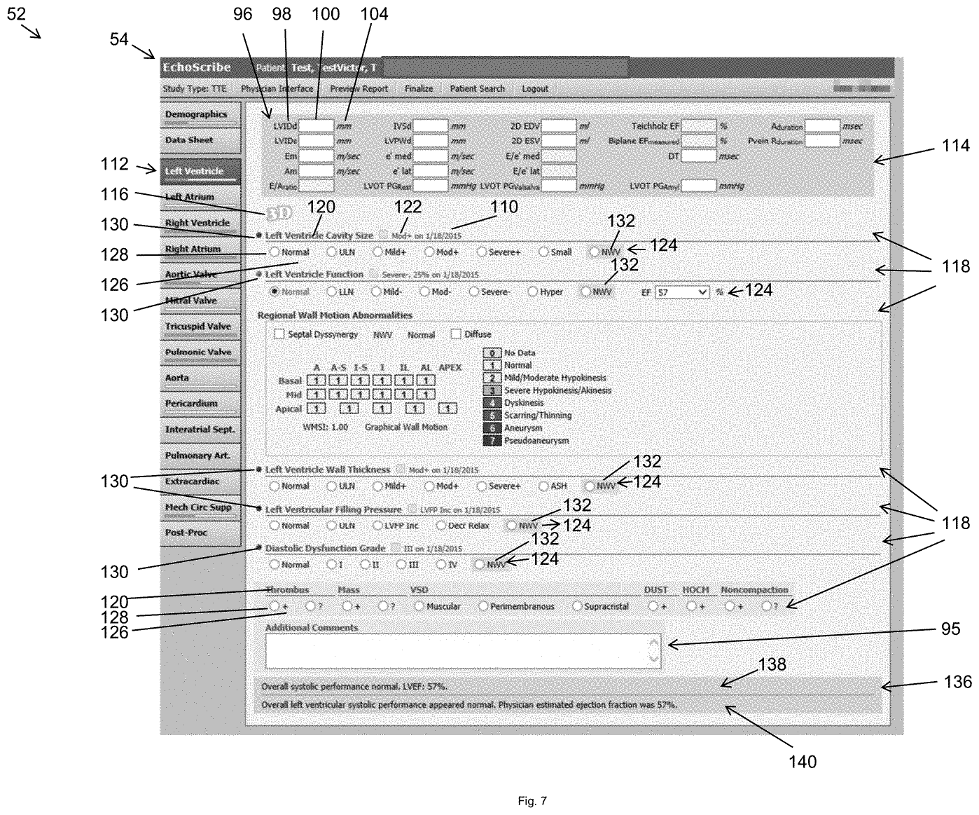

As an example, with reference to FIG. 7, the interactive user page 54 associated with left ventricle tab 112 displays a plurality of evaluation sections 118 entitled “Left Ventricle Cavity Size,” “Left Ventricle Function,” “Regional Wall Motion Abnormalities,” “Left Ventricle Wall Thickness,” “Left Ventricular Filling Pressure,” “Diastolic Dysfunction Grade,” “Thrombus,” “Mass,” “VSD,” “DUST,” “HOCM,” and “Noncompaction” as examples.

As a first example, with reference to the “Left Ventricle Cavity Size” evaluation section 118 which is the upper-most positioned evaluation section 118 on the interactive user page 54 associated with left ventricle tab 112. Adjacent to the descriptor 120 “Left Ventricle Cavity Size” the prior findings indicator 122 is presented which shows “Mod+ on Jan. 18, 2015” as an example. Positioned below a separating line, a plurality of evaluation options 124 are presented including “Normal,” “ULN,” “Mild+,” “Mod+,” “Severe,” and “NWV” each having an input area 128 positioned adjacent the descriptor 126 for selection by cardiologist 18 when appropriate. These evaluation options 124 represent the portfolio of common determinations for this evaluation section 118. The “NWV” evaluation option 124 is selected when cardiologist 18 cannot confidently make a determination based on the data presented in data section 114.

As a second example, with reference to the “Left Ventricle Function” evaluation section 118 which is the second evaluation section 118 on the interactive user page 54 associated with left ventricle tab 112. Adjacent to the descriptor 120 “Left Ventricle Function” the prior findings indicator 122 is presented which shows “Severe-, 25% on Jan. 18, 2015” as an example. This includes a hover function 108, which when hovered over displays the history of these prior findings in a pop-up box. Positioned below a separating line, a plurality of evaluation options 124 are presented including “Normal,” “LLN,” “Mild-,” “Mod-,” “Severe,” “Hyper,” and “NWV” each having an input area 128 positioned adjacent the descriptor 126 for selection by cardiologist 18 when appropriate. Also presented is an evaluation option 124 “EF” (Ejection Fraction) as the descriptor 126 and includes a drop down box as the input area 128 with “%” as the associated units 104. This evaluation option allows cardiologist 18 to select the percentage of “EF”. These evaluation options 124 represent the portfolio of common determinations for this evaluation section 118. The “NWV” evaluation option 124 is selected when cardiologist 18 cannot confidently make a determination based on the data presented in data section 114.

At this point it is important to note that the “EF” or ejection fraction is often the subject of errors from cardiologists when interpreting the results of an echocardiogram. That is, each of the evaluation options 124 “Normal,” “LLN,” “Mild-,” “Mod-,” “Severe,” and “Hyper,” naturally correlate with an ejection fraction range, which is represented as a percentage. When the determination (“Normal,” “LLN,” “Mild-,” “Mod-,” “Severe,” and “Hyper”) of cardiologist 18 does not match or correlate with the ejection fraction percentage applicable to this determination, this discordance this may cast doubt over the validity and reliability of the entire report and/or results as this is an important finding.

As one example:
a determination of “Normal” correlates with an ejection fraction range of 56% to 74%
a determination of “LLN” correlates with and ejection fraction range of 51% to 55%
a determination of “Mild-” correlates with and ejection fraction range of 41% to 50%
a determination of “Mod-” correlates with and ejection fraction range of 31% to 40%
a determination of “Severe” correlates with an ejection fraction range of less than or equal to 30%
a determination of “Hyper” correlates with an ejection fraction range of “75% to 90%

In the arrangement shown, as one example, to help eliminate the possibility that the determination of cardiologist 18 regarding the “Left Ventricle Function” does not match the “EF”, when cardiologist 18, selects an input area 128 next to one of the descriptors 126 of the evaluation options 124 (“Normal,” “LLN,” “Mild-,” “Mod-,” “Severe,” and “Hyper”) the system 10 automatically constrains the possible selections of the “EF” percentage in the drop down box adjacent to the “NWV” evaluation options 124 to present only “EF” percentages that match the selected determination. This eliminates the possibility that the selected determination is discordant with the selected ejection fraction percentage.

In the arrangement shown, as one example, with reference to FIG. 7, when cardiologist 18 selects the input area 128 next to the “Normal” descriptor 126 of the evaluation options 124 the system 10 automatically limits the options available for selection through the drop down box next to “EF” to the range of 56% to 74%. As is show, a “EF” percentage of 57% was selected in this example which is concordant. This constraint of further evaluation options based on prior determinations is repeated throughout this disclosure wherever applicable and/or presented. This feature substantially reduces the error rate in reports 134.

As a third example, with reference to the “Regional Wall Motion Abnormalities” evaluation section 118 which is the third evaluation section 118 on the interactive user page 54 associated with left ventricle tab 112. Positioned within a separating box, a plurality of evaluation options 124 are presented including “Septal Dyssynergy,” “NWV,” “Normal,” and Diffuse” as options. Positioned below these options, a matrix is provided where “Basal,” “Mild,” and “Apical,” are presented along a vertical axis and “A,” “A-S,” “I-S,” “I,” “IL,” and “APEX” are presented along a horizontal axis, as examples. Adjacent this matrix is a key showing 0 as No Data, 1 as Normal, 2 as Mild/Moderate Hypokinesis, 3 as Severe Hypokinesis/Akinesis, 4 as Dyskinesis, 5 as Scarring/Thinning, 6 as Aneurysm and 7 as Pseudoaneurysm, as examples. At each intersection of the matrix, cardiologist 18 may select their determination.

As a fourth example, with reference to the “Left Ventricle Wall Thickness” evaluation section 118 which is the fourth evaluation section 118 on the interactive user page 54 associated with left ventricle tab 112. Adjacent to the descriptor 120 “Left Ventricle Wall Thickness” the prior findings indicator 122 is presented which shows “Mod+ on Jan. 18, 2015” as an example. Positioned below a separating line, a plurality of evaluation options 124 are presented including “Normal,” “ULN,” “Mild+,” “Mod+,” “Severe,” “ASH,” and “NWV” each having an input area 128 positioned adjacent the descriptor 126 for selection by cardiologist 18 when appropriate.

As a fifth example, with reference to the “Left Ventricular Filling Pressure” evaluation section 118 which is the fifth evaluation section 118 on the interactive user page 54 associated with left ventricle tab 112. Adjacent to the descriptor 120 “Left Ventricular Filling Pressure” the prior findings indicator 122 is presented which shows “LVFP Inc on Jan. 18, 2015” as an example. Positioned below a separating line, a plurality of evaluation options 124 are presented including “Normal,” “ULN,” “LVFP Inc,” “Decr Relax,” and “NWV” each having an input area 128 positioned adjacent the descriptor 126 for selection by cardiologist 18 when appropriate.

As a sixth example, with reference to the “Diastolic Dysfunction Grade” evaluation section 118 which is the sixth evaluation section 118 on the interactive user page 54 associated with left ventricle tab 112. Adjacent to the descriptor 120 “Diastolic Dysfunction Grade” the prior findings indicator 122 is presented which shows “III on Jan. 1, 2015” as an example. Positioned below a separating line, a plurality of evaluation options 124 are presented including “Normal,” “I,” “II,” “III,” “IV,” and “NWV” each having an input area 128 positioned adjacent the descriptor 126 for selection by cardiologist 18 when appropriate.

As a seventh example, with reference to the bottom most evaluation section 118 essentially a catch-all evaluation section 118 is presented that includes a plurality of additional evaluation options 124 associated with interactive user page 54 associated with left ventricle tab 112. These additional evaluation options 124 include “Thrombus,” “Mass,” “VSD,” “DUST,” “HICM,” and “Noncompaction” as examples and may be selected when cardiologist 18 determines that these conditions or determinations exist.

In this way, through the presentation of evaluation sections 118 having a descriptor 120, and prior findings indicator 122 along with a plurality of potential evaluation options 124 each having a descriptor 126 and an input area 128, cardiologist 18 can quickly, easily, efficiently and accurately assess the subset of current data set 26 in data section 114 associated with left ventricle tab 112 and make the needed determinations. Cardiologist 18 may make these determinations with the added insight provided by the prior findings indicator 122, as well as further insight provided by hover function 108. Cardiologist 18 may make these determinations with the insight provided by the indications when values 102 in data section 114 vary more than a predetermined amount from that value in prior data set 28, as is described herein. In this way, cardiologist 18 may make not just determinations, but fully informed decisions the historical context of this patient's history in a fast, efficient, easy and accurate manner.

Mandatory Finding Indicator:

In the arrangement shown, to ensure cardiologist 18 makes all needed determinations to comply with applicable evaluation standards, a mandatory finding indicator 130 is positioned adjacent the descriptor 120 of each evaluation section 118. Mandatory finding indicator 130 may be formed of any suitable size, shape and design and indicates that the evaluation section 118 associated with the mandatory finding indicator 130 requires a determination by cardiologist 18 for the review of the echocardiogram to meet applicable evaluation standards. The presence of a mandatory finding indicator 130 indicates to cardiologist 18 that they must make a selection among the plurality of evaluation options 124 to complete the evaluation, even if that selection is “NWV” which indicates that the cardiologist 18 did not feel they had adequate information to make a determination. The selection of evaluation options 124 from evaluation sections 118 with a mandatory finding indicator 130 corresponds with completion reflected in progress bar 80 of tabs 56.

In the arrangement shown, as one example, mandatory evaluation indicator 130 is a button or other visual indication positioned just to the left of the descriptor 120 of evaluation sections 118 that are mandatory. In one arrangement, mandatory evaluation indicator 130 changes when cardiologist 18 has made a determination to indicate satisfaction of the mandatory evaluation indicator 130. As one example, as is shown, mandatory evaluation indicator 130 is red in color prior to a determination being made indicating that a determination needs to be made, and changes to green in color after a determination has been made indicating satisfaction of the mandatory evaluation requirement. Any other type of indication or treatment of evaluation section 118 is hereby contemplated for use to indicate that a determination is mandatory, as well as to indicate whether a mandatory determination has been made, such as bolding the text, highlighting the text, italicizing the text, underlining the text, using a different font, using a unique color, providing a box around evaluation section 118, causing the evaluation section 118 to flash, pulsate or strobe, providing asterisks or other text, or any other treatment. In this way, the presence and use of mandatory finding indicators 130 aid a cardiologist in quickly, efficiently and accurately evaluating the results of an echocardiogram by indicating the most-important, and mandatory, evaluation sections 118 and indicating whether they have been complied with.

Suggested Finding Indicator:

History and medical practice tells us that there is a high correlation between the values 102 of data section 114 and the proper determinations in evaluation sections 118. While the correlation is not perfect between the values 102 of data section 114 and the proper determination in evaluation sections 118, which is why the judgment and experience of cardiologist 18 is required, the correlation is high.

To aid cardiologist in the fast, efficient and accurate review of the current data set 26, in the arrangement shown, as one example, when and where possible, system 10 generates a suggested finding indicator 132 associated with a suggested evaluation option 124. Suggested finding indicator 132 may be formed of any suitable size, shape and design and indicates a suggested determination among evaluation options 124 based on the values in data section 114.

Suggested finding indicator 132 selects the best fitting or most applicable evaluation option 124 among the plurality of evaluation options 124. This is based on current data set 26, prior data sets 28, algorithms, calculations, evaluations, historical data, best practices, current trends, data bases of information of other patients 12, guidelines, artificial intelligence, machine learning, or any other information or calculation or evaluation performed by system 10. In summary, suggested finding indicator 132 is based upon applicable standards and best practices, which are always evolving. The presence of a suggested finding indicator 132 indicates to cardiologist 18 that the system 10, based on the values 102 in data section 114 (and/or current data set 26 and prior data sets 28) and the applicable standards suggests the selection of the indicated evaluation option 124. Cardiologist 18 is not required to select the evaluation option 124 that is associated with suggested finding indicator 132. However, the presence of a suggested finding indicator 132 in association with evaluation sections 118 provides cardiologist 18 with a quick and easily understood suggestion, based on the applicable standards, how the system 10 believes the cardiologist 18 should choose among the evaluation options 124.

The presence of suggested finding indicators 132 has a tendency to provide cardiologist 18 with increased confidence in their selections when their determination corresponds with the suggested finding indicator 132, which can speed the evaluation as well as increase the accuracy of the evaluation. The presence of suggested finding indicators 132 also has a tendency to make cardiologist 18 evaluate their selection with greater scrutiny when determination does not correspond with the suggested finding indicator 132, which can help to prevent errant selections and increase the accuracy of the review. Providing suggested finding indicators 132 also has a tendency to educate cardiologist 18, as well as indicate to cardiologist 18 when they should seek additional opinions and/or suggest conducting further testing.

In the arrangement shown, as one example, suggested finding indicator 132 is indicated by highlighting or shading the evaluation option 124 that the system 10 suggests based upon values 102 in data section 114. Any other type of indication or treatment of evaluation option 124 is hereby contemplated for use as suggested finding indicator 132 to indicate the preferred status of the option, such as bolding the text, highlighting the text, italicizing the text, underlining the text, using a different font, using a unique color, providing a box around evaluation option 124, causing the evaluation option 124 to flash, pulsate or strobe, providing asterisks or other text, or any other treatment. In this way, the presence and use of suggested finding indicators 132 aids cardiologist 18 in quickly, efficiently and accurately evaluating the results of an echocardiogram by indicating the suggested evaluation option 124.

Concordant & Discordant Selection of Evaluation Options:

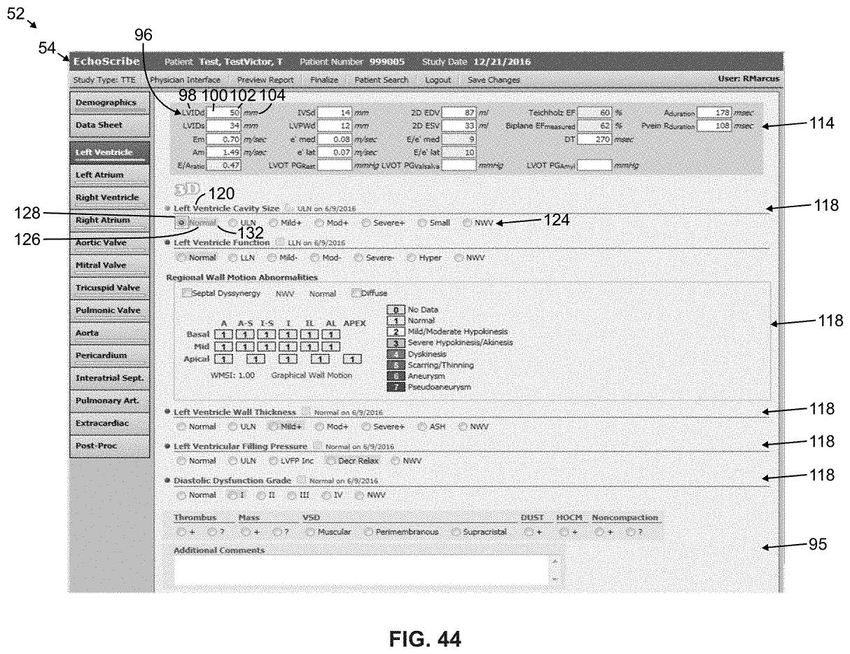

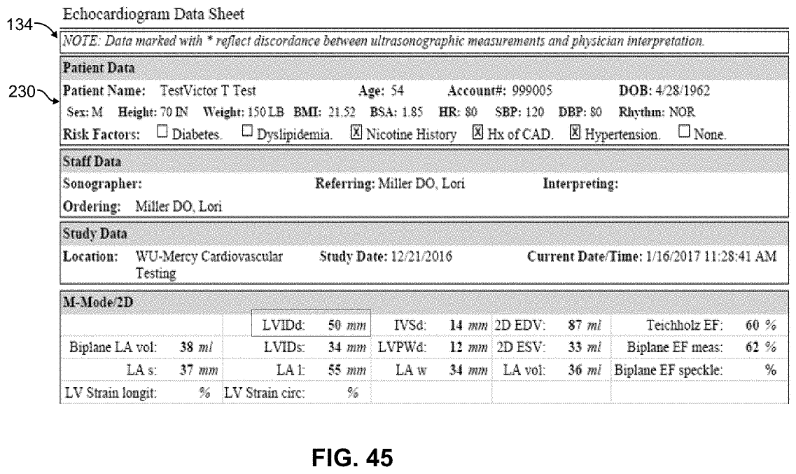

When cardiologist 18 selects an evaluation option 124 from evaluation section 118 that is indicated by a suggested finding indicator 132, this selection is considered “concordant” with the suggested finding indicator 132 and values 102 of data section 114. With reference to FIG. 44 and FIG. 45 a concordant selection is shown.

More specifically, FIG. 44 shows field 96 with descriptor 98 of “LVIDd” having an input area 100 that includes “50” as value 102. The system 10 interprets this value 102 for this field 96 to correspond with the “Normal” evaluation option 124 of the first evaluation section 118. As such, the system 10 associates suggested finding indicator 132 with the “Normal” evaluation option 124. When cardiologist 18 selects input area 128 associated with the “Normal” evaluation option 124, as is shown in FIG. 44, the selection of cardiologist 18 is concordant with the suggestion of system 10, as is indicated by suggested finding indicator 132. In the resulting report 134, as is shown in-part in FIG. 45, the determination of cardiologist 18 is concordant with the result depicted on report 134, which is “LVIDd; 50 mm”. There is no confusion with this report 134 as the opinion of cardiologist 18 is concordant with the value 102 of data section 114 and the suggested finding indicator 132 of system 10. This value 102 is indicated as concordant on report 134 as no asterisk or other text treatment or other condition is indicated in association with the value 102.

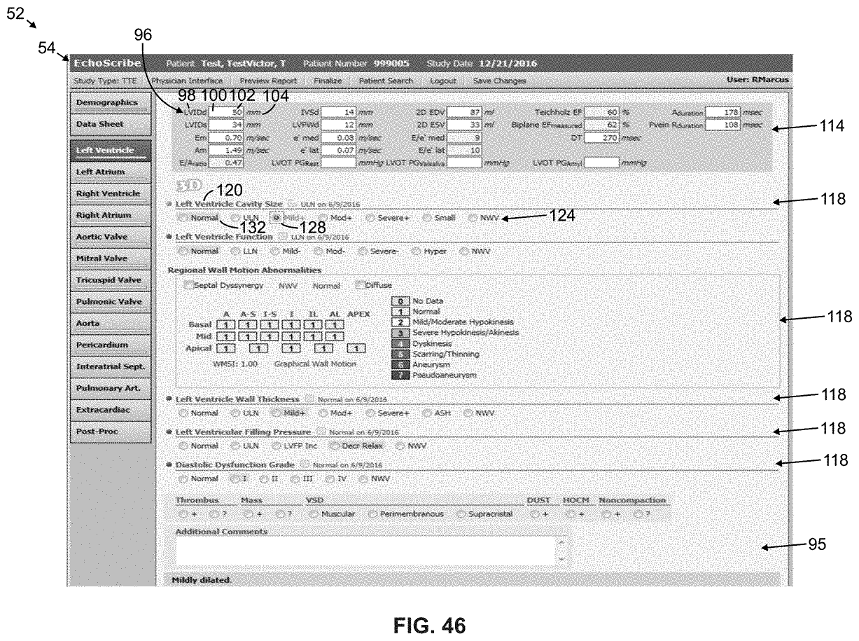

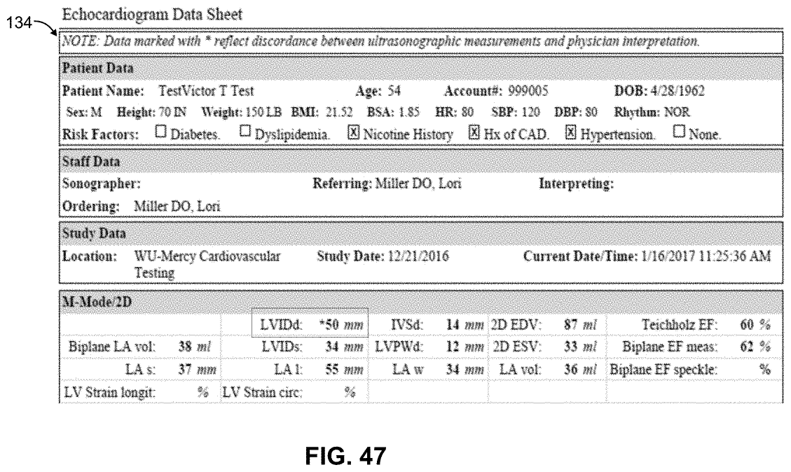

When cardiologist 18 selects an evaluation option 124 from evaluation section 118 that is not indicated by a suggested finding indicator 132, this selection is considered “discordant” with the suggested finding indicator 132 and values 102 of data section 114. With reference to FIG. 46 and FIG. 47 a discordant selection is shown.

More specifically, FIG. 46 shows field 96 with descriptor 98 of “LVIDd” having an input area 100 that includes “50” as value 102. The system 10 interprets this value 102 for this field 96 to correspond with the “Normal” evaluation option 124 of the first evaluation section 118. As such, the system 10 associates suggested finding indicator 132 with the “Normal” evaluation option 124. When cardiologist 18 selects an input area 128 other than the “Normal” evaluation option 124, as is shown in FIG. 46, where cardiologist 18 selected the evaluation option 124 associated with “Mild+”, the selection of cardiologist 18 is discordant with the suggestion of system 10, as is indicated by suggested finding indicator 132. In the resulting report 134, as is shown in-part in FIG. 47, the determination of cardiologist 18 is discordant with the result depicted on report 134, which is “LVIDd; 50 mm”. This leads to confusion with this report 134 as the opinion of cardiologist 18 is in-conflict with, or is discordant with, the generally accepted interpretation of the value 102 of data section 114 and the suggested finding indicator 132 of system 10. To avoid any confusion, this value 102 is indicated as discordant in report 134 as an asterisk or other text treatment or other condition is indicated in association with the value 102.

To ensure proper interpretation of report 134, at the top of report 134 it states “NOTE: Data marked with * reflect discordance between ultrasonographic measurements and physician interpretation.” Without this indication and acknowledgement of discordance, a later reviewer of report 134 is left to guess which of the conflicting information is correct, the value 102 or the determination by cardiologist 18 (which are discordant with one another). As such, the acknowledgement of discordance strengthens the findings of the report 134 and eliminates conflicting information.

Comments Section:

In the arrangement shown, as one example, positioned below the plurality of evaluations sections 118, a comments section 95 is presented. In this arrangement, comments section 95 allows cardiologist 18 to add or dictate commentary to this portion of display page 54 associated with left ventricle tab 112. The information added or dictated may then be saved and added to a report 134 which is contemplated herein. In the arrangement shown, comments section 95 includes a free-form fillable field.

Summary Section:

In the arrangement shown, as one example, positioned below the comments section 95 a summary section 136 is presented. Summary section 136 is formed of any suitable size, shape and design and is configured to provide a summary of the findings of cardiologist 18 associated with the left ventricle tab 112. In the arrangement shown, as one example, summary section 136 includes an overall impression section 138 and a detailed findings section 140. In the arrangement shown, as one example, system 10 automatically generates the text description for the overall impression section 138 and a detailed findings section 140 based upon the selections made by cardiologist 18 when completing their review of the interactive user page 54 associated with left ventricle tab 112.

In the arrangement shown, as one example, overall impression section 138 and a detailed findings section 140 are configured to be clear, concise and easily read and understood text-based descriptions of the findings of cardiologist 18. In the arrangement shown, as one example, overall impression section 138 of summary section 136 is configured to be a high-level summary of the overall impression of cardiologist; whereas detailed findings section 140 of summary section 136 is configured to have a higher level of detail as compared to the overall impression section 138. In the arrangement shown, as one example, overall impression section 138 and detailed findings section 140 are contained within a shaded area that provides a clearly visible boundary and are separated by a line that extends between the overall impression section 138 and detailed findings section 140 thereby providing a clear separation between the overall impression section 138 and detailed findings section 140.

In the arrangement shown, as one example, the notes in overall impression section 138 are included in an overall impression 142 of report 134. Similarly, in the arrangement shown, as one example, the notes in detailed findings section 140 are included in the detailed findings of report 134. Upon review of the overall impression 142 of report 134 a reviewer gets a high-level summary view of the determination of cardiologist 18. Upon review of the detailed findings 144 of report 134 a reviewer gets a more-in-depth summary view of the determination of cardiologist 18.

The notes of the overall impression section 138 are culled to the highest valued comments, or the most important notes regarding the determinations of the current data set 26. In contrast, the notes in detailed findings section 140 are intended to include further detail that provides insight into the determinations related to current data set 26. These notes of both overall impression section 138 and detailed findings section 140 are generated through the iterative revision and contributive authorship of experts in the field with insight, experience and practical knowledge in the field. Which notes to include based upon the selections by cardiologist are the result of the application of software, artificial intelligence and/or machine learning and are continuously improved with each use of the system 10. In the event that cardiologist 18 disagrees with the notes of overall impression section 138 and detailed findings section 140 cardiologist 18 may change their selections, or alternatively cardiologist 18 may enter their own notes in additional comments section 95.

In one arrangement, the comments entered by cardiologist 18 into comment section 95 are also repeated in the overall impression 142 of report 134. In another arrangement, the comments entered by cardiologist 18 into comment section 95 are also repeated in the detailed findings 144 of report 134. In yet another arrangement, the comments entered by cardiologist 18 into comment section 95 are also repeated in their own portion of report 134 or any other portion or portions of report 134.

The automatic generation of the comments in overall impression section 138 and detailed findings section 140 based on the selections made by cardiologist 18 from the pre-selected evaluation options 124 of evaluation sections 118 provides a great number benefits. That is, the automatic generation of the comments in comments in overall impression section 138 and detailed findings section 140, saves cardiologist 18 the time of generating these notes, eliminates the possibility of human error when generating these notes, has the effect of standardizing the resulting reports 134 between various cardiologists 18, has the effect of being more thorough, increases accuracy, and makes it easier and faster for the cardiologist 18 to review the echocardiogram. As such, the automatic generation of the comments in overall impression section 138 and detailed findings section 140 provide a great number of benefits.

Other Tabs:

The left ventricle tab 112 was described in detail above. A plurality of other tabs 56 are presented in system 10 that include many of the same features as left ventricle tab 112, while being directed to different portions or aspects of the heart of patient 12. For purposes of brevity unless described otherwise, the disclosure related to left ventricle tab 112 is incorporated into and/or is applicable to other tabs 56. This includes left atrium tab 148, right ventricle tab 150, right atrium tab 152, aortic valve tab 154, mitral valve tab 156, tricuspid valve tab 158, pulmonic valve tab 160, aorta tab 162, pericardium tab 164, interatrial septum tab 166, pulmonary artery tab 168, extracardiac tab 170, mechanical circulation support tab 172 and post-procedure tab 174, among others. That is, these other tabs 56 include some or all of the same features, layout and functionality as left ventricle tab 112 and as such description related to left ventricle tab 112 is applicable to these other tabs 56 as well.

Left Atrium Tab:

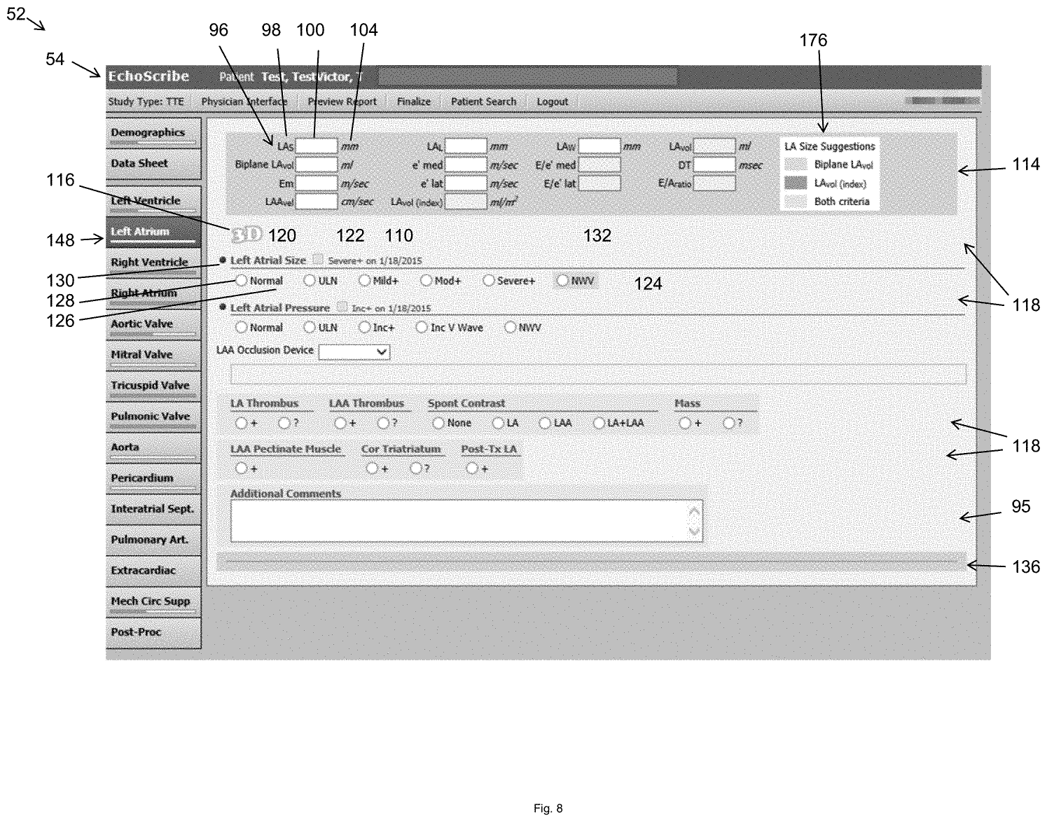

In the arrangement shown, as one example, with reference to FIG. 8, interactive user display 52 includes a left atrium tab 148. Left atrium tab 148 may be formed of any suitable size, shape, or design and serves to indicate to the user what subset of data set 26, 28 the tab 56 relates to, which in this case is information from the performance of an echocardiogram on patient 12 that relates to the left atrium of the heart of patient 12. In one arrangement, as is shown, left atrium tab 148 is positioned just below the left ventricle tab 112. In the arrangement shown, as one example, left atrium tab 148 is the fourth interactive user page 54 a cardiologist 18 is taken to, after completing review of the left ventricle tab 112. However cardiologist 18 may freely move among interactive user pages 54 by selecting tabs 56.

Multiple Suggested Findings:

While left atrium tab 148 includes much of the same or similar functionality and structure presented with respect to left ventricle tab 112, left atrium tab 148 includes a key 176 that relates to suggested finding indicator 132. Suggested finding indicator 132 is designed to indicate to cardiologist 18 the suggested evaluation option 124 based upon applicable evaluation standards, guidelines, rules, best practices, or other applicable information. In some situations, however, more than one set of evaluation criteria apply to the determination of the best-fit evaluation option 124 for suggested finding indicator 132. When more than one applicable evaluation standards apply to the selection of the best-fit evaluation option 124 for suggested finding indicator 132 there can be discordance as the application of different standards can lead to the selection of different evaluation options 124.

In the arrangement shown, as one example of where multiple suggested finding indicators 132 are applicable, key 176 describes “LA Size Suggestions” using two different applicable evaluation standards, “Biplane LA_{VOL}” and “LA_{VOL (index)}”. Key 176 associates a color of shading with each of “Biplane LA_{VOL}” and “LA_{VOL (Index)}”. Key 176 also includes a color of shading where both criteria (“Both criteria”) arrive at the same conclusion. In this way, when both applicable evaluation standards, “Biplane LA_{VOL}” and “LA_{VOL (index)}”, suggest the same evaluation option 124, the cardiologist 18 understands the concordance among the two applicable evaluation standards by the suggested finding indicator 132 being presented in the treatment (colored shading) associated with “Both criteria.” In this way, when the applicable evaluation standards, “Biplane LA_{VOL}” and “LA_{VOL (index)}”, suggest the different evaluation options 124, the cardiologist 18 understands the discordance among the two applicable evaluation standards by one suggested finding indicator 132 being presented in the treatment (colored shading) associated with “Biplane LA_{VOL}” and ne suggested finding indicator 132 being presented in the treatment (colored shading) associated with “LA_{VOL (Index)}.” In this discordant example, cardiologist 18 must choose one evaluation option thereby violating the suggestion of at least one of the applicable evaluation standards. In this way, the use of key 176 and the use of multiple applicable evaluation standards to produce multiple suggested finding indicators 132 provides cardiologist 18 with greater insight into the proper or suggested interpretation of the data set 26.

Right Ventricle Tab:

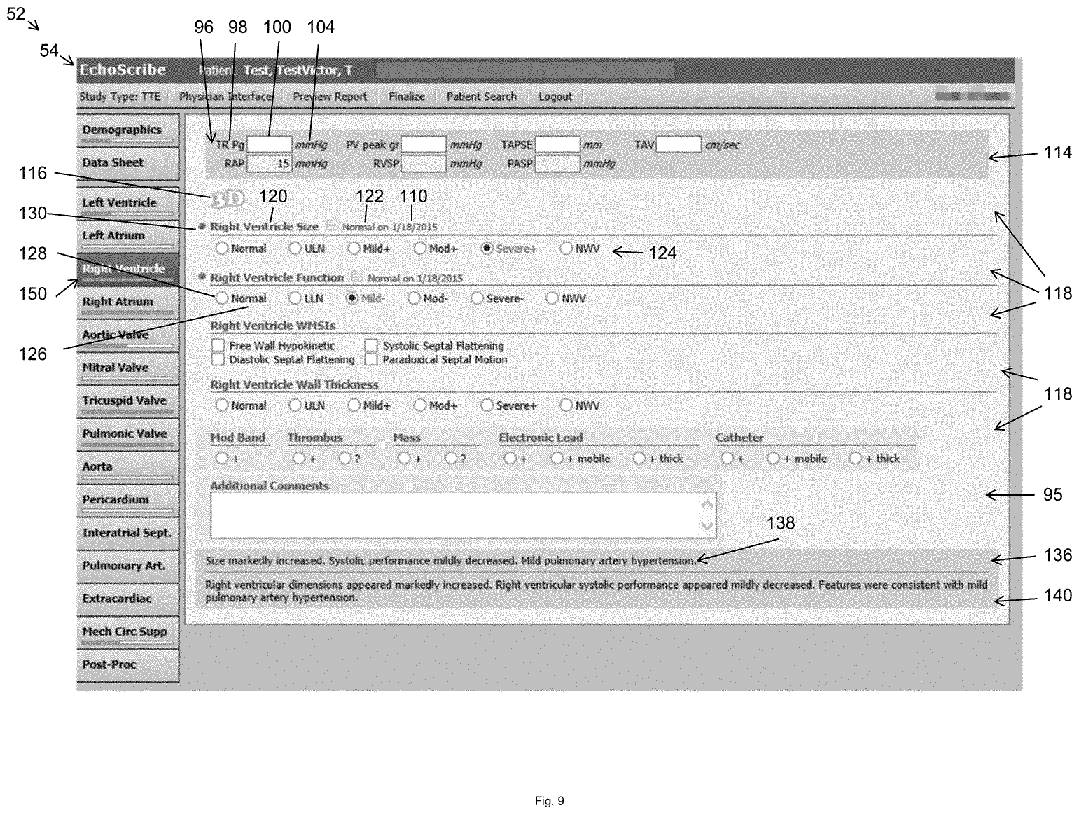

In the arrangement shown, as one example, with reference to FIG. 9, interactive user display 52 includes a right ventricle tab 150. Right ventricle tab 150 may be formed of any suitable size, shape, or design and serves to indicate to the user what subset of data set 26, 28 the tab 56 relates to, which in this case is information from the performance of an echocardiogram on patient 12 that relates to the right ventricle of the heart of patient 12. In one arrangement, as is shown, right ventricle tab 150 is positioned just below the left atrium tab 148. In the arrangement shown, as one example, right ventricle tab 150 is the fifth interactive user page 54 a cardiologist 18 is taken to, after completing review of the left atrium tab 148. However cardiologist 18 may freely move among interactive user pages 54 by selecting tabs 56.

Right Atrium Tab:

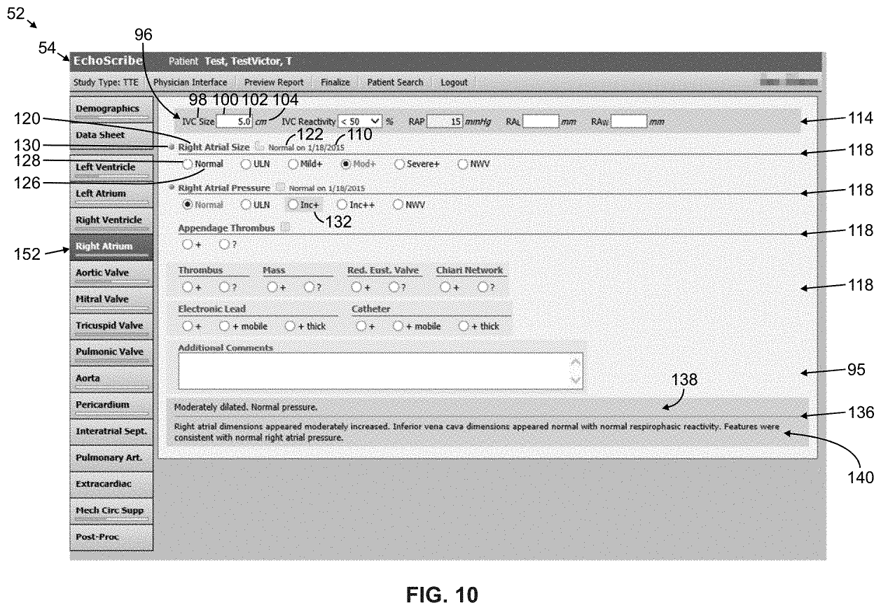

In the arrangement shown, as one example, with reference to FIG. 10, interactive user display 52 includes a right atrium tab 152. Right atrium tab 152 may be formed of any suitable size, shape, or design and serves to indicate to the user what subset of data set 26, 28 the tab 56 relates to, which in this case is information from the performance of an echocardiogram on patient 12 that relates to the right atrium of the heart of patient 12. In one arrangement, as is shown, right atrium tab 152 is positioned just below the right ventricle tab 150. In the arrangement shown, as one example, right atrium tab 152 is the sixth interactive user page 54 a cardiologist 18 is taken to, after completing review of the right ventricle tab 150. However cardiologist 18 may freely move among interactive user pages 54 by selecting tabs 56.

Aortic Valve Tab:

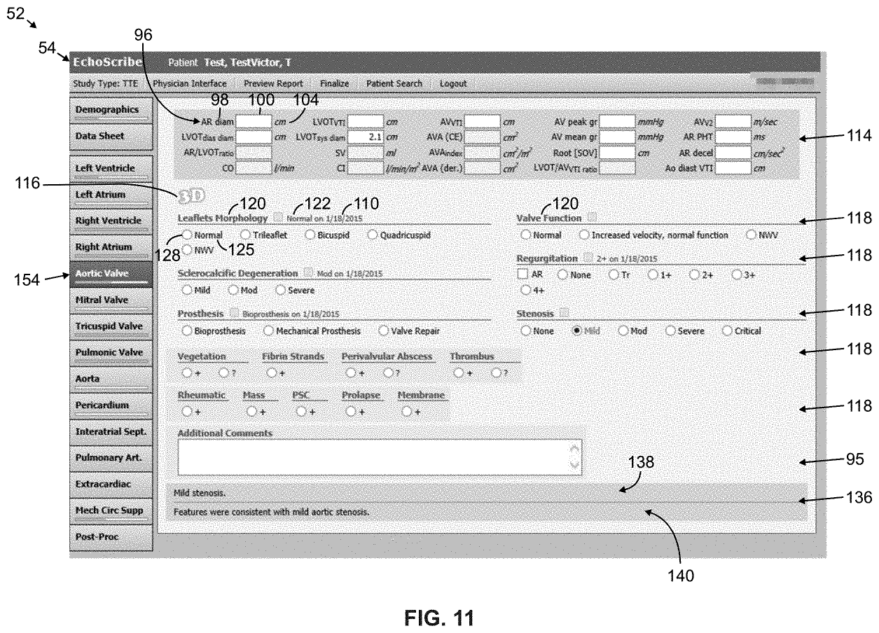

In the arrangement shown, as one example, with reference to FIG. 11, interactive user display 52 includes an aortic valve tab 154. Aortic valve tab 154 may be formed of any suitable size, shape, or design and serves to indicate to the user what subset of data set 26, 28 the tab 56 relates to, which in this case is information from the performance of an echocardiogram on patient 12 that relates to the aortic valve of the heart of patient 12. In one arrangement, as is shown, aortic valve tab 154 is positioned just below the right atrium tab 152. In the arrangement shown, as one example, aortic valve tab 154 is the seventh interactive user page 54 a cardiologist 18 is taken to, after completing review of the right atrium tab 152. However cardiologist 18 may freely move among interactive user pages 54 by selecting tabs 56.

Mitral Valve Tab:

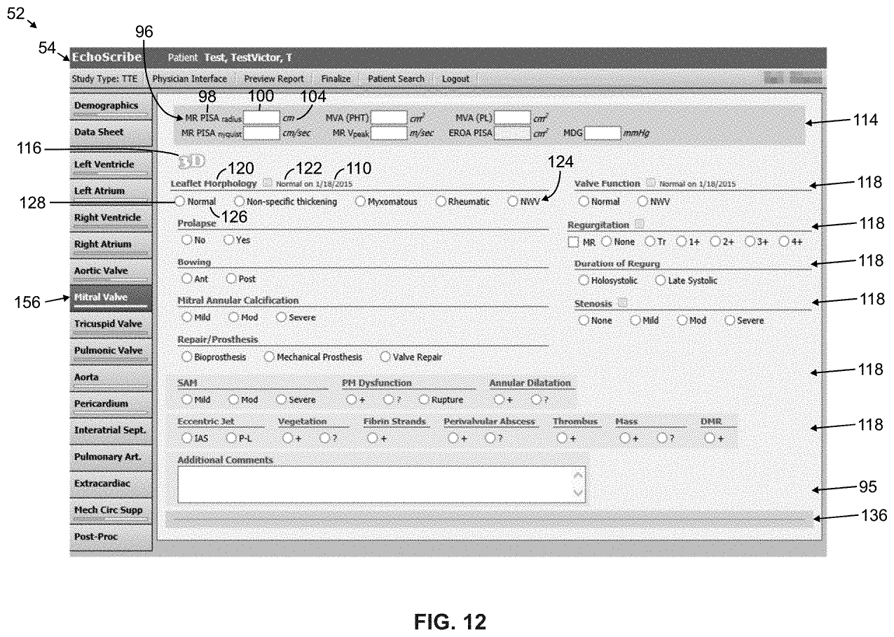

In the arrangement shown, as one example, with reference to FIG. 12, interactive user display 52 includes an mitral valve tab 156. Mitral valve tab 156 may be formed of any suitable size, shape, or design and serves to indicate to the user what subset of data set 26, 28 the tab 56 relates to, which in this case is information from the performance of an echocardiogram on patient 12 that relates to the mitral valve of the heart of patient 12. In one arrangement, as is shown, mitral valve tab 156 is positioned just below the aortic valve tab 154. In the arrangement shown, as one example, mitral valve tab 156 is the eighth interactive user page 54 a cardiologist 18 is taken to, after completing review of the aortic valve tab 154. However cardiologist 18 may freely move among interactive user pages 54 by selecting tabs 56.

Tricuspid Valve Tab:

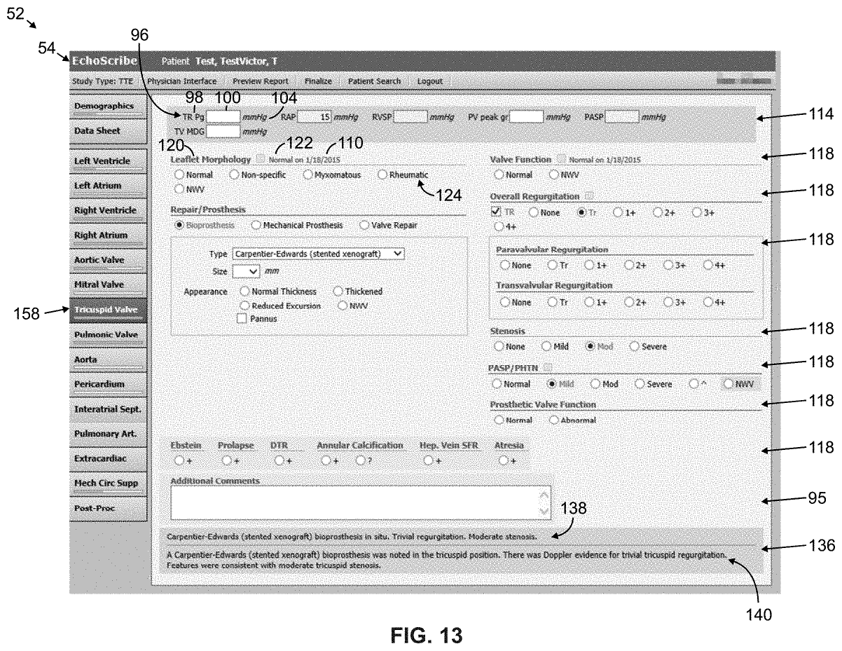

In the arrangement shown, as one example, with reference to FIG. 13, interactive user display 52 includes a tricuspid valve tab 158. Tricuspid valve tab 158 may be formed of any suitable size, shape, or design and serves to indicate to the user what subset of data set 26, 28 the tab 56 relates to, which in this case is information from the performance of an echocardiogram on patient 12 that relates to the tricuspid valve of the heart of patient 12. In one arrangement, as is shown, tricuspid valve tab 158 is positioned just below the mitral valve tab 156. In the arrangement shown, as one example, tricuspid valve tab 158 is the ninth interactive user page 54 a cardiologist 18 is taken to, after completing review of the mitral valve tab 156. However cardiologist 18 may freely move among interactive user pages 54 by selecting tabs 56.

Pulmonic Valve Tab:

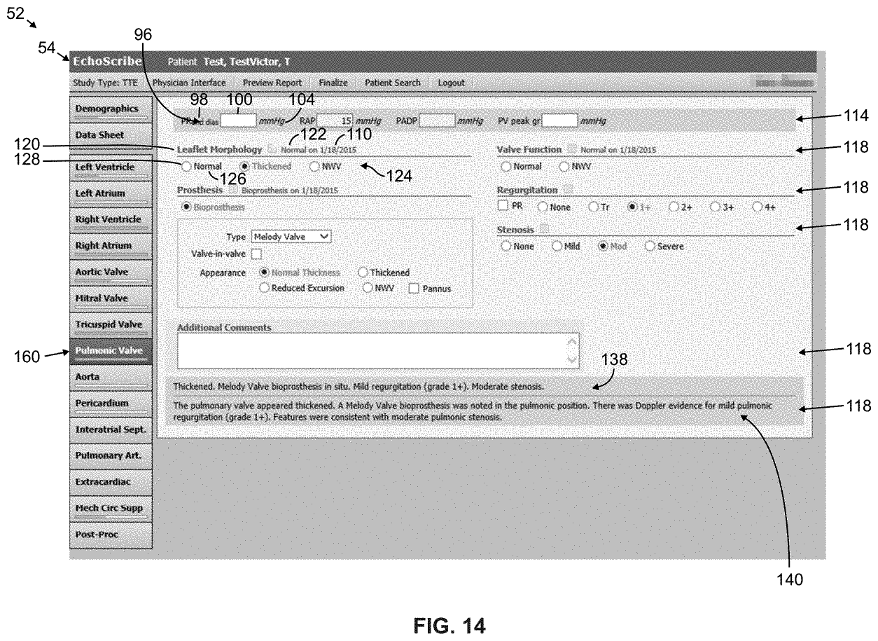

In the arrangement shown, as one example, with reference to FIG. 14, interactive user display 52 includes a pulmonic valve tab 160. Pulmonic valve tab 160 may be formed of any suitable size, shape, or design and serves to indicate to the user what subset of data set 26, 28 the tab 56 relates to, which in this case is information from the performance of an echocardiogram on patient 12 that relates to the pulmonic valve of the heart of patient 12. In one arrangement, as is shown, pulmonic valve tab 160 is positioned just below the tricuspid valve tab 158. In the arrangement shown, as one example, pulmonic valve tab 160 is the tenth interactive user page 54 a cardiologist 18 is taken to, after completing review of the tricuspid valve tab 158. However cardiologist 18 may freely move among interactive user pages 54 by selecting tabs 56.

Aorta Tab:

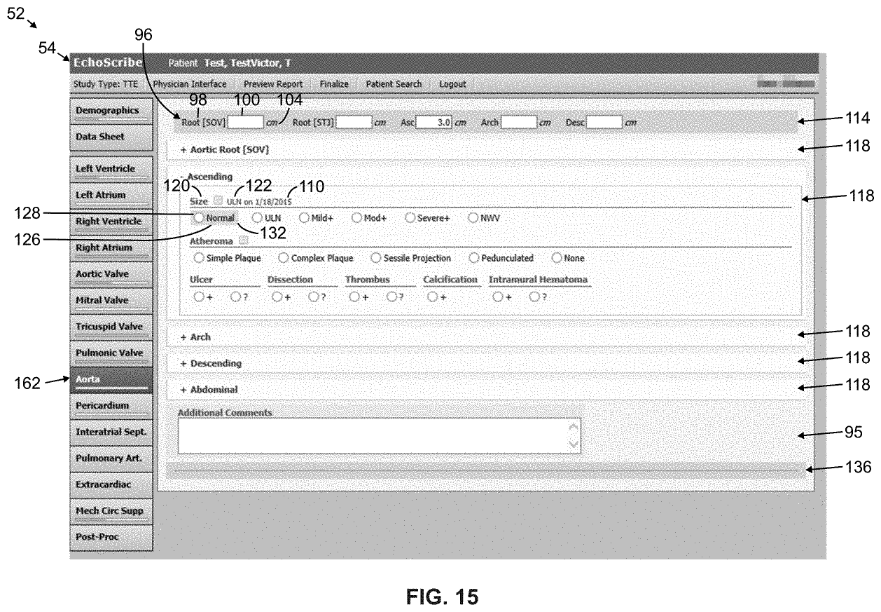

In the arrangement shown, as one example, with reference to FIG. 15, interactive user display 52 includes an aorta tab 162. Aorta tab 162 may be formed of any suitable size, shape, or design and serves to indicate to the user what subset of data set 26, 28 the tab 56 relates to, which in this case is information from the performance of an echocardiogram on patient 12 that relates to the aorta of the heart of patient 12. In one arrangement, as is shown, aorta tab 162 is positioned just below the pulmonic valve tab 160. In the arrangement shown, as one example, aorta tab 162 is the eleventh interactive user page 54 a cardiologist 18 is taken to, after completing review of the pulmonic valve tab 160. However cardiologist 18 may freely move among interactive user pages 54 by selecting tabs 56.

Pericardium Tab:

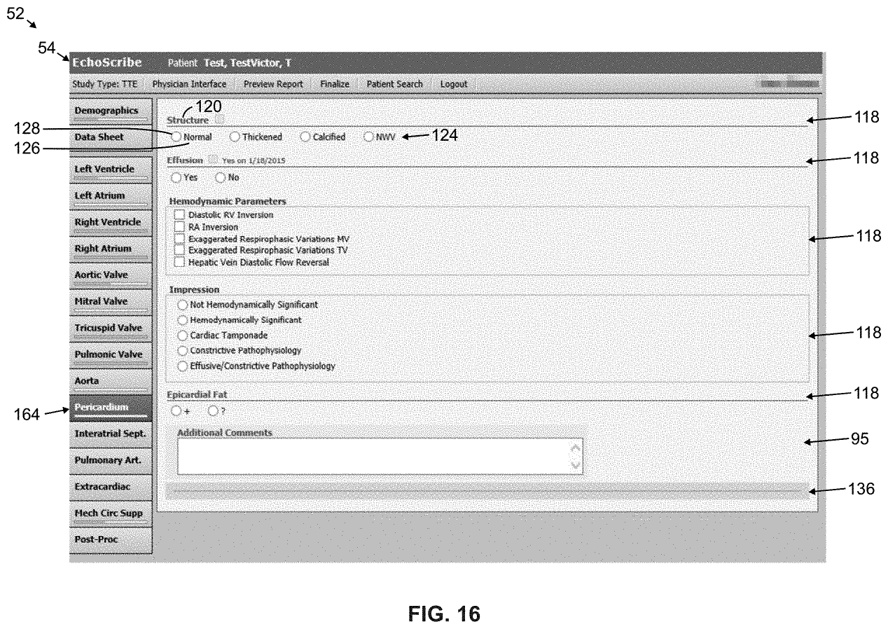

In the arrangement shown, as one example, with reference to FIG. 16, interactive user display 52 includes a pericardium tab 164. Pericardium tab 164 may be formed of any suitable size, shape, or design and serves to indicate to the user what subset of data set 26, 28 the tab 56 relates to, which in this case is information from the performance of an echocardiogram on patient 12 that relates to the pericardium of the heart of patient 12. In one arrangement, as is shown, pericardium tab 164 is positioned just below the aorta tab 162. In the arrangement shown, as one example, pericardium tab 164 is the twelfth interactive user page 54 a cardiologist 18 is taken to, after completing review of the aorta tab 162. However cardiologist 18 may freely move among interactive user pages 54 by selecting tabs 56.

Interatrial Septum Tab:

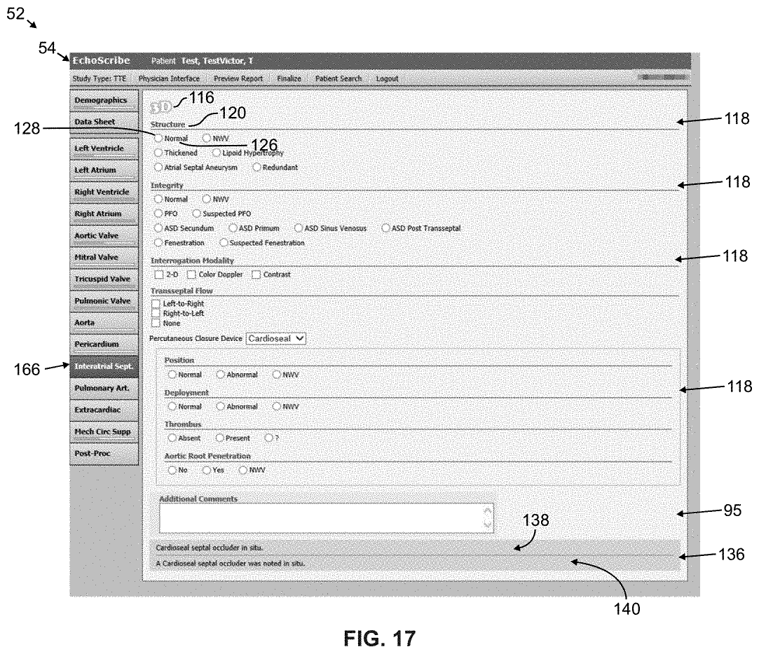

In the arrangement shown, as one example, with reference to FIG. 17, interactive user display 52 includes a interatrial septum tab 166. Interatrial septum tab 166 may be formed of any suitable size, shape, or design and serves to indicate to the user what subset of data set 26, 28 the tab 56 relates to, which in this case is information from the performance of an echocardiogram on patient 12 that relates to the interatrial septum of the heart of patient 12. In one arrangement, as is shown, interatrial septum tab 166 is positioned just below the pericardium tab 164. In the arrangement shown, as one example, interatrial septum tab 166 is the thirteenth interactive user page 54 a cardiologist 18 is taken to, after completing review of the pericardium tab 164. However cardiologist 18 may freely move among interactive user pages 54 by selecting tabs 56.

Pulmonary Artery Tab:

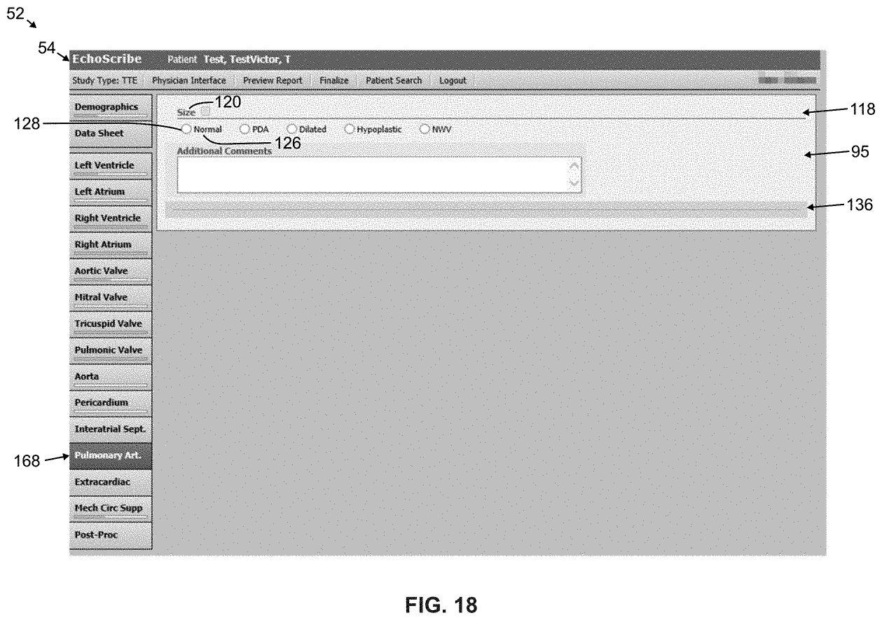

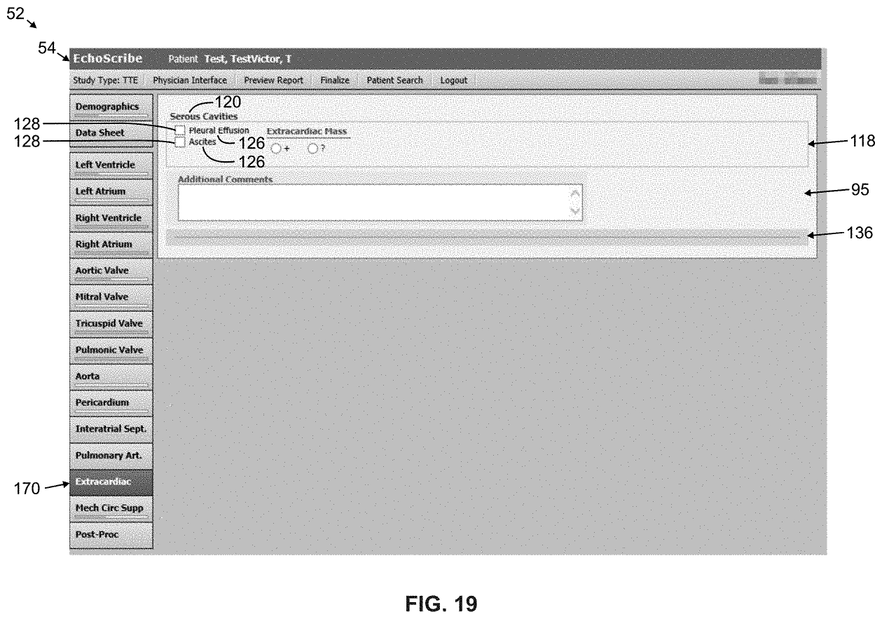

In the arrangement shown, as one example, with reference to FIG. 18, interactive user display 52 includes a pulmonary artery tab 168. Pulmonary artery tab 168 may be formed of any suitable size, shape, or design and serves to indicate to the user what subset of data set 26, 28 the tab 56 relates to, which in this case is information from the performance of an echocardiogram on patient 12 that relates to the pulmonary artery of the heart of patient 12. In one arrangement, as is shown, pulmonary artery tab 168 is positioned just below the interatrial septum tab 166. In the arrangement shown, as one example, pulmonary artery tab 168 is the fourteenth interactive user page 54 a cardiologist 18 is taken to, after completing review of the interatrial septum tab 166. However cardiologist 18 may freely move among interactive user pages 54 by selecting tabs 56.

Extracardiac Tab:

In the arrangement shown, as one example, with reference to FIG. 19, interactive user display 52 includes an extracardiac tab 170. Extracardiac tab 170 may be formed of any suitable size, shape, or design and serves to indicate to the user what subset of data set 26, 28 the tab 56 relates to, which in this case is information from the performance of an echocardiogram on patient 12 that relates to the extracardiac area of the heart of patient 12. In one arrangement, as is shown, extracardiac tab 170 is positioned just below the pulmonary artery tab 168. In the arrangement shown, as one example, extracardiac tab 170 is the fifteenth interactive user page 54 a cardiologist 18 is taken to, after completing review of the pulmonary artery tab 168. However cardiologist 18 may freely move among interactive user pages 54 by selecting tabs 56.

Mechanical Circulatory Support Tab:

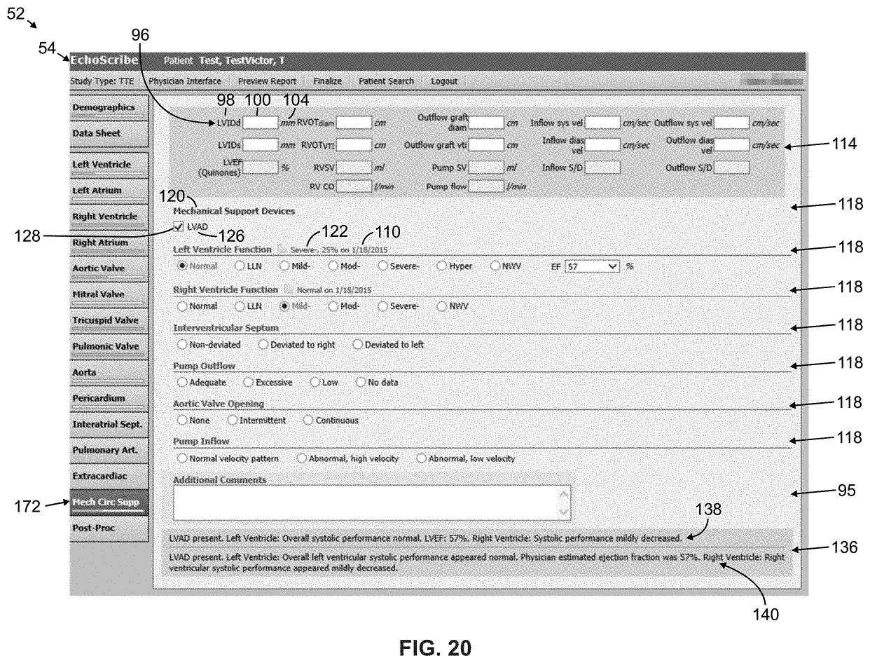

In the arrangement shown, as one example, with reference to FIG. 20, interactive user display 52 includes a mechanical circulatory support tab 172. Mechanical circulatory support tab 172 may be formed of any suitable size, shape, or design and serves to indicate to the user what subset of data set 26, 28 the tab 56 relates to, which in this case is information from the performance of an echocardiogram on patient 12 that relates to a mechanical circulatory support system associated with the heart of patient 12. In one arrangement, as is shown, mechanical circulatory support tab 172 is positioned just below the extracardiac tab 170. In the arrangement shown, as one example, mechanical circulatory support tab 172 is the sixteenth interactive user page 54 a cardiologist 18 is taken to, after completing review of the extracardiac tab 170. However cardiologist 18 may freely move among interactive user pages 54 by selecting tabs 56.

Post-Procedure Tab:

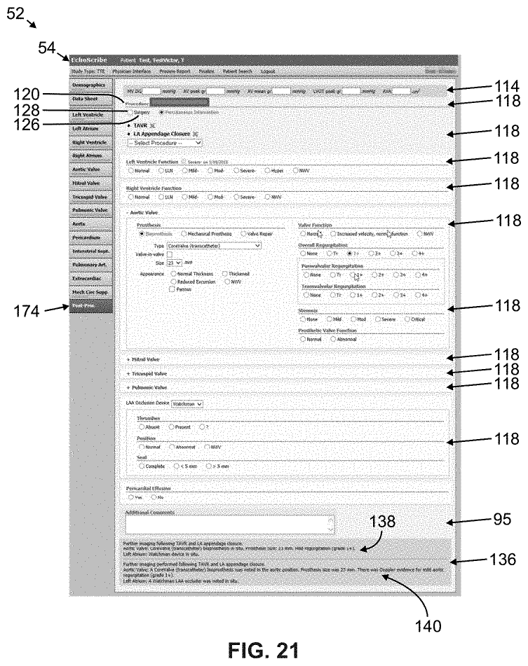

In the arrangement shown, as one example, with reference to FIG. 21, interactive user display 52 includes a post-procedure tab 174. Post-procedure tab 174 may be formed of any suitable size, shape, or design and serves to indicate to the user what subset of data set 26, 28 the tab 56 relates to, which in this case is information from the performance of an echocardiogram on patient 12 that relates to post-procedure information associated with the heart of patient 12. In one arrangement, as is shown, post-procedure tab 174 is positioned just below the mechanical circulatory support tab 172. In the arrangement shown, as one example, post-procedure tab 174 is the seventeenth interactive user page 54 a cardiologist 18 is taken to, after completing review of the mechanical circulatory support tab 172. However cardiologist 18 may freely move among interactive user pages 54 by selecting tabs 56.

Finalize Study:

In the arrangement shown, if cardiologist 18 goes through tabs 56 in chronological order, as they are laid out on interactive user page 54, post-procedure tab 174 is the last tab 56 cardiologist 18 fills out prior to finalizing their review of data set 26. However, cardiologist 18 is not required to move through tabs 56 in the order they are laid out. Instead, cardiologist 18 may move through tabs 56 and make the required determinations and non-mandatory determinations in any order. In doing so, cardiologist 18 is aided by the presence of progress bars 80 in tabs 56 which quickly and easily show how much of the required findings have been completed and with this information cardiologist 18 may move through the tabs 56 to complete the review of data set 26.

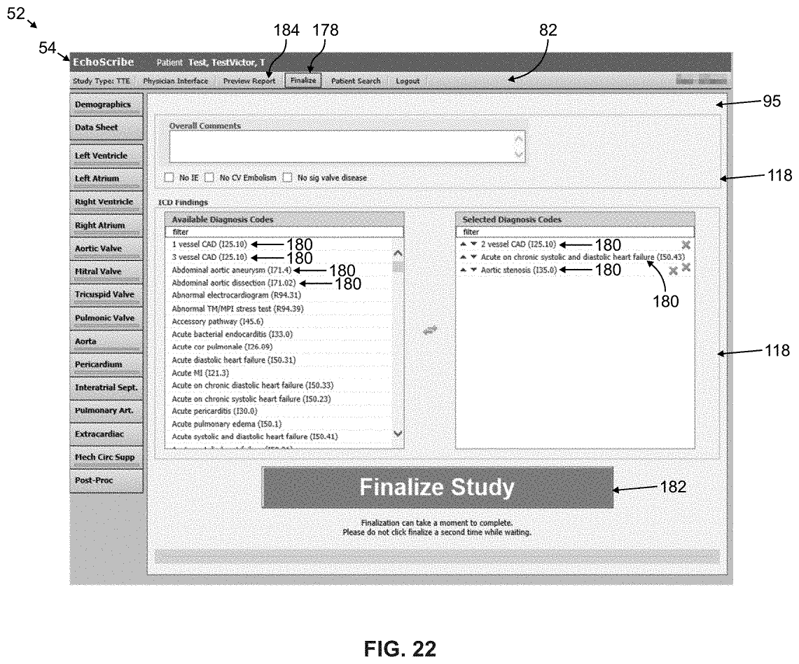

In one arrangement, when cardiologist 18 has completed their review of data set 26, as is indicated by all progress bars 80 being complete, as is shown in FIG. 22, cardiologist 18 may select the “Finalize” button 178 from menu tab 82. In one arrangement, when cardiologist 18 has completed all of their mandatory findings the Finalize button 178 is illuminated with a treatment indicating that cardiologist has completed all of their required findings and they can complete their study at any time. In one arrangement, this treatment to Finalize button 178 includes illuminating Finalize button 178 in red bolded text and treatment, however any other form of a treatment is hereby contemplated for use such as leaving the Finalize button 178 in greyed-out treatment indicating that it cannot be selected until all mandatory findings have been made and converting the Finalize button 178 to non-greyed-out text once all mandatory findings have been made indicating that it can now be selected. Other treatments may include, flashing, pulsing, bolding, highlighting or any other treatment indicating that the Finalize button 178 may be selected. Even when all mandatory findings have been made and Finalize button 178 is selectable, cardiologist may continue their review to refine their opinion and results.

In the arrangement shown, as one example, when the Finalize button 178 from menu tab 82 is selected the interactive user page 54 shown in FIG. 22 is presented wherein cardiologist 18 may enter “Overall Comments” related to the review of current data set 25 in comment section 95; cardiologist 18 may select overall findings through evaluation section 118 positioned below the comment section 95; and cardiologist 18 may select various diagnosis codes 180 from a predetermined list of diagnosis codes 180 that are applicable to patient 12. In the arrangement shown, as one example, when a diagnosis code 180 is selected it moves from an “Available Diagnosis Code” field to a “Selected Diagnosis Codes” field.

Once cardiologist 18 is satisfied with their review of current data set 26 and their associated findings, cardiologist 18 selects the Finalize Study Button 182 which generates report 134. Again, cardiologist 18 is prevented from completing their review of the current data set 26 until they make a finding for all evaluation sections 118 that are mandatory, even if that finding is “NWV” indicating cardiologist 18 was unable to make a determination. In this way this prevents the submission of incomplete reports 134 or reports that do not meet the applicable standards for complete review.

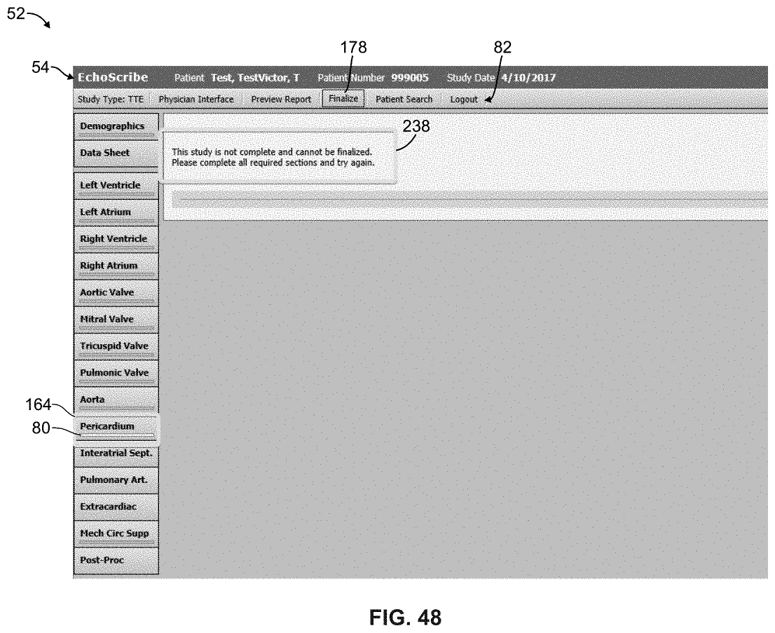

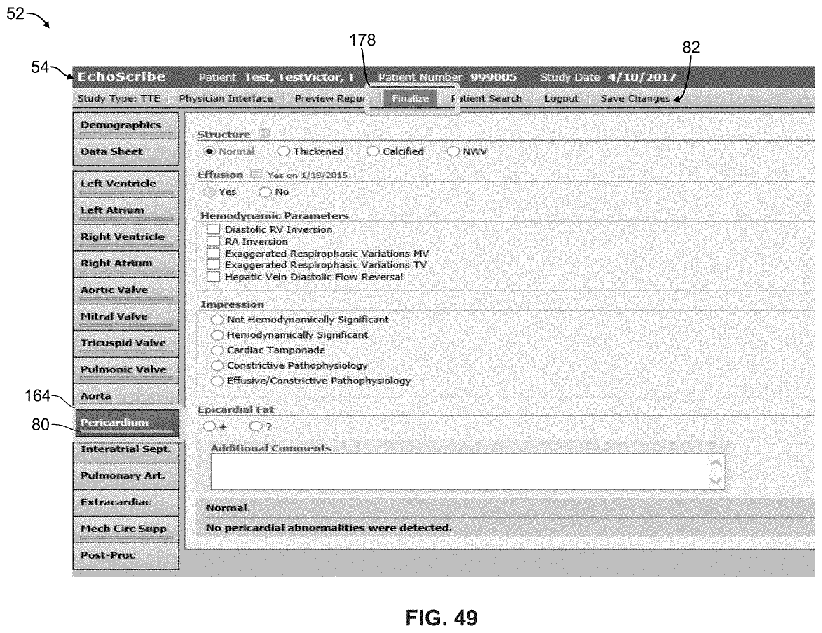

In the arrangement shown, as one example, with reference to FIG. 48, if cardiologist 18 attempts to finalize a report by selecting Finalize button 178 when all of the mandatory findings have not yet been made, a pop-up box 238 is presented that indicates findings need to be made to complete the submission of the study. In one arrangement, when cardiologist 18 is presented with pop-up box 238 all cardiologist 18 must do is look to tabs 56 to see what progress bars 80 are not complete. In this arrangement, progress bar 80 associated with pericardium tab 164 remains open. With reference to FIG. 49, once the mandatory findings of pericardium tab 164 are made, the Finalize button 178 is presented in a treatment indicating that it can now be selected, which corresponds to progress bar 80 in pericardium tab 164 being completed.

In another arrangement, the tabs 56 that have mandatory findings that have yet to be made are presented in a treatment, such as red bolding, pulsing, flashing or the like, that indicates that cardiologist 18 must visit these tabs 56 and make further findings. In one arrangement, when cardiologist 18 visits these tabs 56 the mandatory findings that have yet to be made are presented in similar treatment such as red bolding, pulsing, flashing or the like, that indicates that cardiologist 18 must make these determinations before finalizing the review.

Preview Report:

At any point during the review of current data set 26, cardiologist 18 may select the Preview Report button 184 which will present cardiologist 18 with a preview of report 34. The preview of report 34 provides cardiologist 18 with insight into how the report 34 will look according to their selections and comments made during their review of current data set 26.

Report:

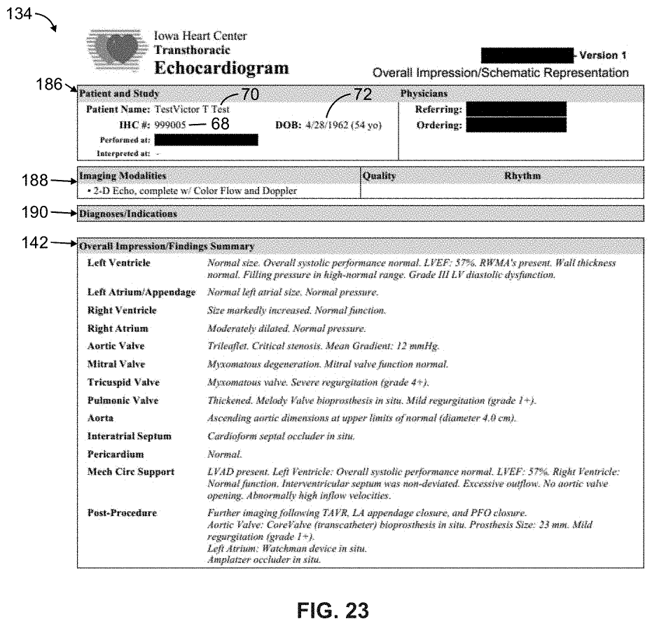

One of the benefits of system 10 is that it automatically generates a comprehensive report 134 when cardiologist 18 completes their review of current data set 26. In the arrangement shown, as one example, an example report 134 is presented with respect to FIG. 23 through FIG. 27.

With reference to FIG. 231n the arrangement shown, as one example, report 134 includes a patient and study section 186 that includes patient name 70, date of birth 72, patient number 68 and other demographic information of patient 12 and information about the performance of the echocardiogram as well as its interpretation.

In the arrangement shown, as one example, report 134 includes an imaging modalities section 188 that includes information regarding the imaging methods used.

In the arrangement shown, as one example, report 134 includes a diagnoses/indications section 190 that includes information regarding the diagnoses and indications that the review of current data set 26 revealed. Or, said another way, diagnoses/indications section 190 includes a summary of the diagnoses and indications that cardiologist 18 determined exist.

Overall Impression:

In the arrangement shown, as one example, report 134 includes an overall impression 142. Overall impression 142 includes summary comments regarding the determinations made by cardiologist 18 in each tab 56. In one arrangement, overall impression 142 of report 134 includes each of the comments from the overall impression section 138 of summary section 136 from each of the tabs 56. In this way, overall impression 142 consolidates the overall impression sections 138 from each tab 56 in a single section of report 134. In the arrangement shown, as one example, overall impression 142 is organized by tab 56, with the comments from the overall impression section 138 of each tab 56 positioned next to the name of each tab 56. In this way, a reviewer can get a comprehensive understanding of the review of current data set 18 by cardiologist 18 by reviewing the overall impression 142. In the arrangement shown, as one example, tabs 56 of overall impression 142 are organized in the same order as they are presented on interactive user pages 54. This maintains consistency between report 134 and the interactive user pages 54 of system 10.

In one arrangement, a comment is included in overall impression 142 for each evaluation section 118 that requires a finding and therefore includes a mandatory finding indicator 130. In another arrangement, a comment is included in overall impression 142 for each evaluation 118 that cardiologist 18 made a finding for, whether it was a mandatory finding or a voluntary or non-mandatory finding. In another agreement, system 10 populates overall impression 142 with comments based on a decision matrix as to what is most important and valuable based on the selections made by cardiologist 18 which may include some or all of the mandatory findings and voluntary findings.

Schematic Representation:

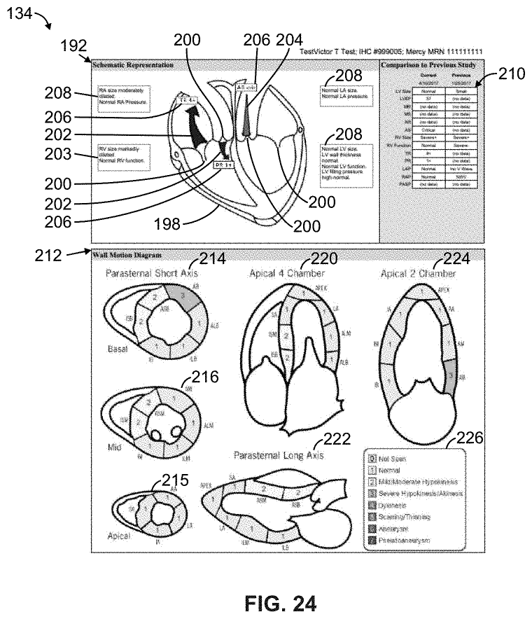

With reference to FIG. 24, in the arrangement shown, as one example, report 134 includes schematic representation 192. Schematic representation 192 is formed of any suitable size, shape and design and is configured to provide a quickly, easily and clearly understood diagram of the heart of patient 12 along with symbols and summaries of the determinations of cardiologist 18.

In the arrangement shown, as one example, the schematic representation 192 includes a demonstrative heart diagram 198 which includes a representation of each of the four valves 200 and chamber characteristics 208. Common heart conditions that relate to valves 200 include regurgitation, which is where the valve 200 fails to fully close thereby causing blood to leak through the valve 200; and stenosis, which is hardening of the valve 200 or restriction of the valve 200 which reduces flow through the valve 200. In the arrangement shown, as one example, an arrow symbol 202 is used to depict regurgitation and a cone symbol 204 is used to depict stenosis, however any other form of a symbol is hereby contemplated for use. In the arrangement shown, arrow symbol 202 is colored blue to depict regurgitation and cone symbol 204 is colored red to depict stenosis, however any other configuration is hereby contemplated for use. As the severity of the condition, regurgitation or stenosis, increases, the size of the arrow symbol 202 or cone symbol 204 increases. When the valve 200 is operating properly, no symbol 202, 204 is present with respect to valve 200. When a symbol 202, 204 is present in association with a valve 200, a summary 206 is positioned at the end of symbol 202, 204 that describes the condition and/or the severity of the condition.

Also shown in schematic representation 192 is an overall summary of each chamber (LV, LA, RV, RA that describes the characteristics of that chamber (chamber characteristics 208) and provides a summary of findings for each valve 200.

In the arrangement shown, as one example, positioned adjacent the schematic representation 192 is a comparison 210 of current findings to previous findings for various aspects of the heart of patient 12 along with the date of the findings. In the arrangement shown, as one example, these findings include “LV Size,” “LVEF,” “MR,” “MS,” “AR,” “AS,” “RV Size,” “RV Function,” “TR,” “PR,” “LAP,” “RAP,” “PASP” as examples.

Presenting this information in schematic representation 192 provides the reviewer with a quick, efficient, accurate and easily understood overall understanding of the condition of the heart of patient 12.

Wall Motion Diagram:

With reference to FIG. 24, in the arrangement shown, as one example, report 134 includes wall motion diagram 212. Wall motion diagram 212 is formed of any suitable size, shape and design and is configured to provide a quickly, easily and clearly understood diagram showing the wall motion of the heart of patient 12 along with numerical values which describe the wall motion of portions of the heart.

In the arrangement shown, as one example, the wall motion diagram 212 includes a plurality of demonstrative diagrams showing portions of the wall of the heart of patient 12. These demonstrative diagrams include Parasternal Short Axis Basal diagram 214, Parasternal Short Axis Mid diagram 216, Parasternal Short Axis Apical diagram 218, Apical 4 Chamber diagram 220, Parasternal Long Axis diagram 222, and Apical 2 Chamber diagram 224. Each of these diagrams 214, 216, 218, 220, 222, 224 show the wall of a heart which is separated into a plurality of segments with each segment including the determination of cardiologist 18 from the Regional Wall Motion Abnormalities evaluation section 18, shown on FIG. 7, which is a value from zero to seven. In the arrangement shown, as one example, wall motion diagram 212 includes a key 226 which shows an interpretation of each of these values with zero being “Not Seen,” one being “Normal,” two being “Mild/Moderate Hypokinesis,” three being “Severe Hypokenisis/Akinesis,” four being “Dykensis,” five being “Scarring/Thinning,” six being “Aneurysm,” and seven being “Pseudoaneurysm” with each of these having their own corresponding color which shades regions of the diagrams 214, 216, 218, 220, 222, 224 that have been indicated with this determination. Presenting key 226 on wall motion diagram 212 provides the reviewer with a quick reference to ensure the reviewer understands the determinations of cardiologist 18 for each of the regions of the wall of the heart of patient 12.

Presenting this information in diagrams 214, 216, 218, 220, 222, 224 provides the reviewer with a quick, efficient, accurate and easily understood overall understanding of the wall motion of the heart of patient 12. These diagrams 214, 216, 218, 220, 222, 224 quickly convey in a convenient manner where particular areas of the heart have wall motion issues.

Regional Wall Motion Abnormalities Section:

In the arrangement shown, as one example, report 134 includes a regional wall motion abnormalities section 228. regional wall motion abnormalities section 228 includes summary comments regarding the determinations made by cardiologist 18 regarding abnormalities in the regional wall motion of the heart of patient 12. In one arrangement, regional wall motion abnormalities section 228 summarizes the information depicted in wall motion diagram 212.

Detailed Findings:

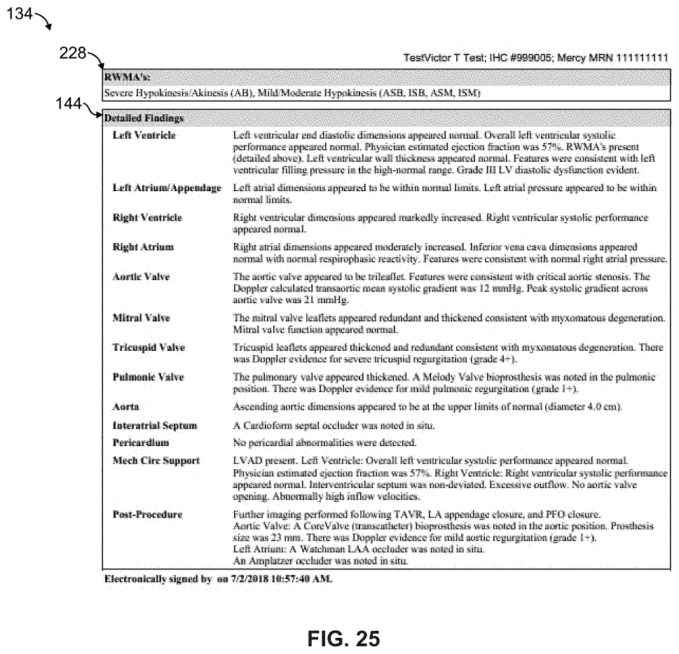

In the arrangement shown, as one example, with reference to FIG. 25 report 134 includes detailed findings 144. Detailed findings 144 include detailed summary comments regarding the determinations made by cardiologist 18 in each tab 56. These comments include greater detail and insight as compared to the comments in overall impression 142. In one arrangement, detailed findings 144 of report 134 include each of the comments from the detailed findings section 140 of summary section 136 from each of the tabs 56. In this way, detailed findings 144 consolidate the detailed findings section 140 from each tab 56 in a single section of report 134. In the arrangement shown, as one example, detailed findings 144 is organized by tab 56, with the comments from the detailed findings section 140 of each tab 56 positioned next to the name of each tab 56. In this way, a reviewer can get a comprehensive understanding of the review of current data set 26 by cardiologist 18 by reviewing the detailed findings 144. In the arrangement shown, as one example, tabs 56 of detailed findings 144 are organized in the same order as they are presented on interactive user pages 54. This maintains consistency between report 134 and the interactive user pages 54 of system 10.

In one arrangement, a comment is included in detailed findings 144 for each evaluation section 118 that requires a finding and therefore includes a mandatory finding indicator 130. In another arrangement, a comment is included in detailed findings 144 for each evaluation 118 that cardiologist 18 made a finding for, whether it was a mandatory finding or a voluntary or non-mandatory finding. In another agreement, system 10 populates detailed findings 144 with comments based on a decision matrix as to what is most important and valuable based on the selections made by cardiologist 18 which may include some or all of the mandatory findings and voluntary findings.

Data Sheet:

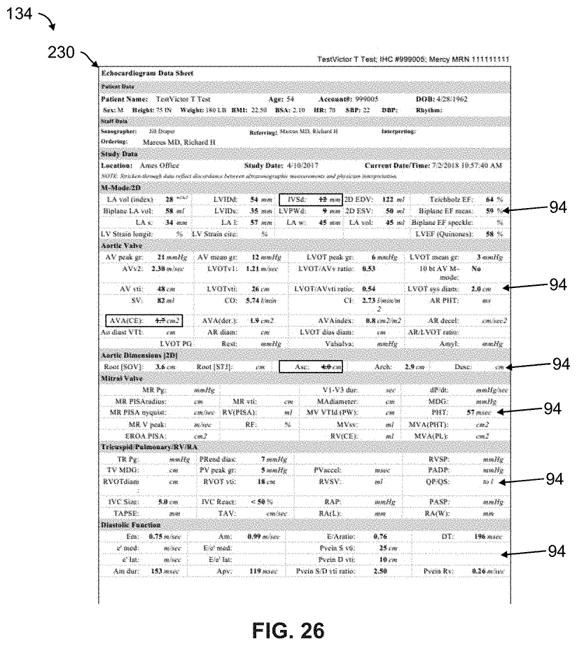

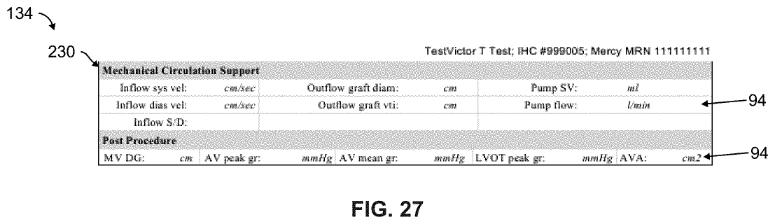

In the arrangement shown, as one example, with reference to FIG. 26 and FIG. 27, report 134 includes a data sheet 230. Data sheet 230 is a summary representation of current data set 26. In the arrangement shown, as one example, data sheet 230 includes the information presented in interactive user page 54 associated with data sheet tab 106. That is, data sheet 230 includes demographic data related to patient 12 well as the current data set 26 separated into sections 94 of the echocardiogram sections 94 that include a plurality of fields 96 having descriptors 98, input areas 100 that receive values 104 and have units 104 associated therewith. The system 10 auto-populates values 102 into the input areas 100 with information from data set 26 as well as manipulations and findings made by cardiologist 18. Organizing, packaging and displaying related information on data sheet 230 in this manner in a standardized, consolidated and convenient manner aids later reviewers of data sheet 230 in their difficult task of quickly, accurately and thoroughly reviewing and interpreting the information in data sheet 230

The data sheet 230 has a similar layout and appearance to the manner in which current data set 26 is laid out on the interactive user page 54 associated with data sheet tab 106. That is because the purposes of interactive user page 54 associated with data sheet tab 106 and data sheet 230 are similar. The purpose of interactive user page 54 associated with data sheet tab 106 is to convey the information of current data sheet 26 to cardiologist 18. The purpose of data sheet 230 is to convey the information of current data sheet 230 as well as the findings of cardiologist 18 to a later reviewer, such as a treating physician 14. As such, for purposes of brevity unless described otherwise, the disclosure related to interactive user page 54 associated with data sheet tab 106 is incorporated into and/or is applicable to data sheet 230.

One item of note is that data sheet 230 provides an indication when the findings of cardiologist 18 are discordant with the generally accepted interpretation of values 102 of current data set 26 based on applicable standards. When this discordance occurs, values 102 are indicated by a treatment, such as highlighting, bolding, boxing, asterisks, strike-through text, colored text, or another treatment. In the arrangement shown, as one example, data sheet 230 states: “NOTE: Stricken-through data reflect discordance between ultrasonographic measurements and physician interpretation.” As such, as is shown, the values 102 that are shown in stricken-through text indicate a discordance between the finding of cardiologist 18 and the values 102 of the echocardiogram. This appears three times on FIG. 26, for “IVSd: mm,” “AVA(CE): cm2,” and “Asc: cm” as examples. By indicating on data sheet 230 that there is a discordance between the finding of cardiologist 18 and the values 102 of current data set 26 provide a reviewer of the data sheet 230 with a higher level of confidence that the discordance was intentional and not in error.

In addition to these features, like the interactive user page 54 associated with data sheet tab 106, data sheet 230 may include: indications when the values 102 of the current data set 26 vary more than a predetermined amount from values 102 of the prior data set 28; indications when a value 102 is a calculated value; the hover function 108, as well as any other feature or function presented herein.

Organizing, packaging and displaying related information on data sheet 230, as well as the other portions of report 134, in a standardized, consolidated and convenient manner aids later reviewers of data sheet 230, and report 134, in their difficult task of quickly, accurately and thoroughly reviewing and interpreting the information in data sheet 230 and report 134.

Critical Finding Contact Requirement:

In one arrangement, when cardiologist 18 completes their review of current data set 26, when a critical finding is made, system 10 notifies cardiologist 18 to immediately contact treating physician 14, patient 12 and/or another healthcare professional associated with patient 12 or another person associated with patient 12. This is because the critical finding requires immediate attention and the health and safety of patient 12 may be in jeopardy. In one arrangement, system 10 requires cardiologist 18 to call treating physician 14, patient 12 and/or another healthcare professional associated with patient 12 or another person associated with patient 12 before they can complete the report 134 and/or review of current data set. This ensures that when a critical finding is made the information is provided to treating physician 14, patient 12 and/or another healthcare professional associated with patient 12 or another person associated with patient 12 as soon as possible.

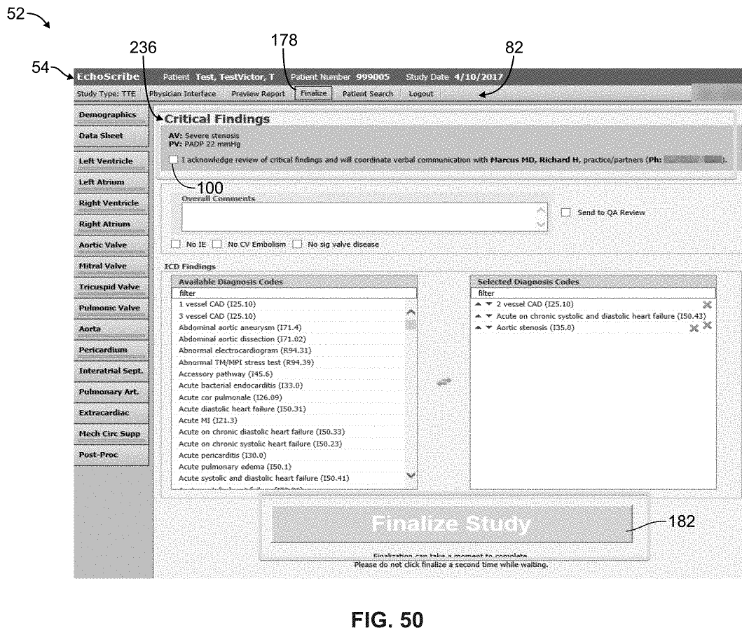

In the arrangement shown, as one example, with reference to FIG. 50, when Finalize button 178 is selected by cardiologist 18 and a critical finding has been made, a critical finding alert 236 is presented to cardiologist 18 indicating to cardiologist 18 that they must call and/or make verbal communication with the treating physician 14 before they can complete the study. In this verbal communication, cardiologist 18 is to relay the information regarding the critical finding to the treating physician 14. In the arrangement shown, as one example, critical finding alert 236 is presented at the top of the interactive user page 54 associated with Finalize button 178 of menu tabs 82 indicating the critical finding, which in the arrangement shown is “AV: Severe stenosis” and “PV: PADP 22 mmHg”. Also shown is an input area 100 that requires cardiologist 18 to check off before the study can be finalized. Next to this input area 100, as one example, the critical finding alert 236 states “I acknowledge review of critical findings and will coordinate verbal communication with . . . ” and includes a name and phone number of the contact for patient 12. Also shown is that the finalize study button 182 cannot be selected until the input area 100 of critical finding alert 236 has been completed/selected.

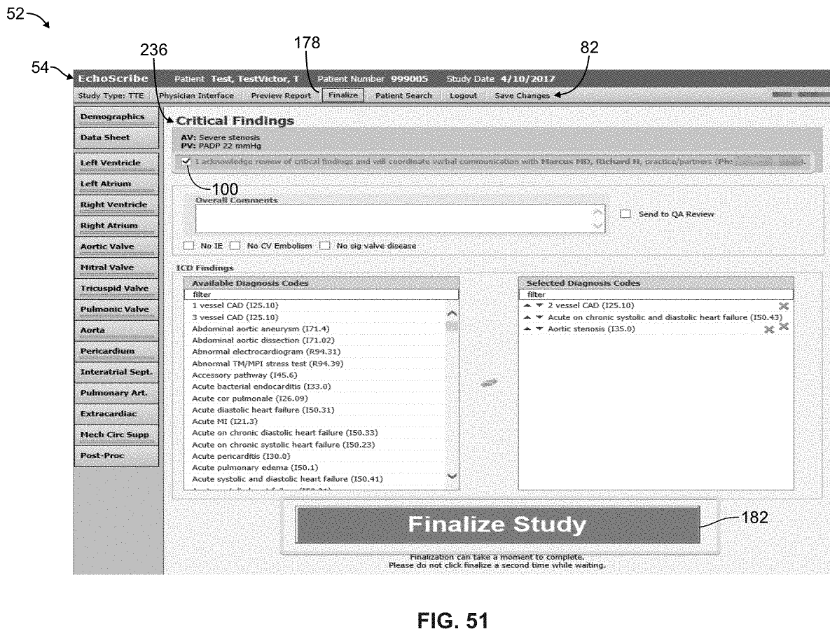

With reference to FIG. 51 this shows completion of the input area of critical finding alert 236 which shows that the finalize study button 182 is active and may be selected.

Schematic Representation Examples:

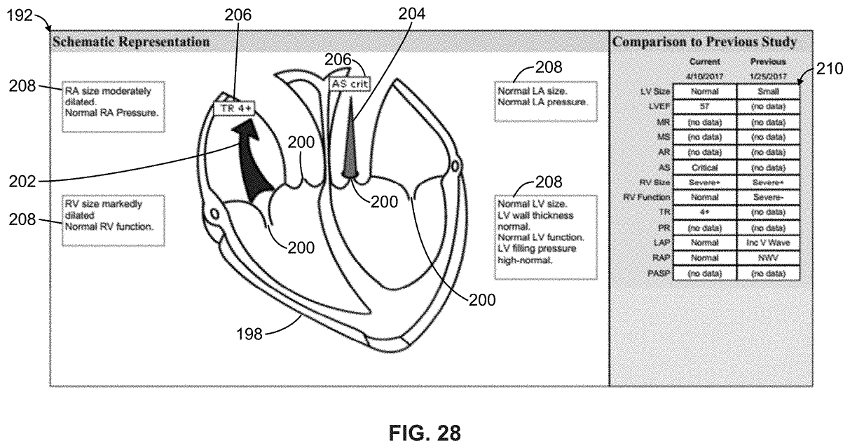

With reference to FIG. 28, a schematic representation 192 from report 134 is presented. This schematic representation 192 shows a critical aortic stenosis and 4+ tricuspid regurgitation. This is a critical finding that would invoke the immediate contact requirement.

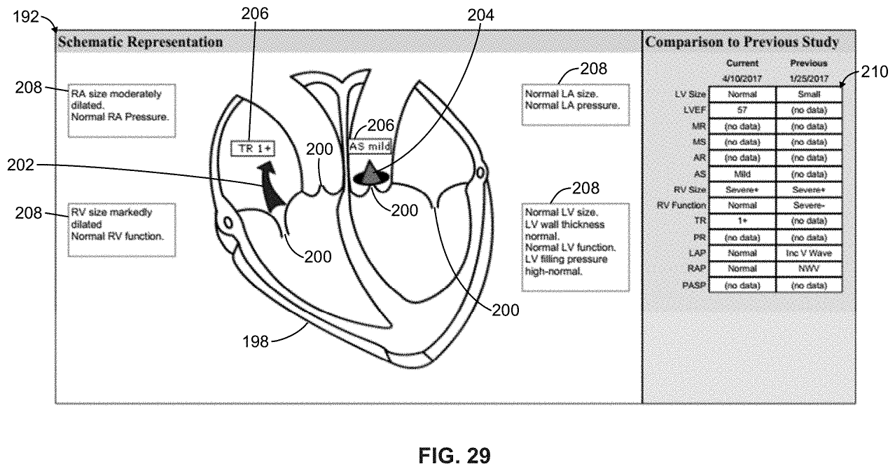

With reference to FIG. 29, a schematic representation 192 from report 134 is presented. This schematic representation 192 shows a mild aortic stenosis and 1+ tricuspid regurgitation. This finding would not invoke the immediate contact requirement associated with a critical finding.

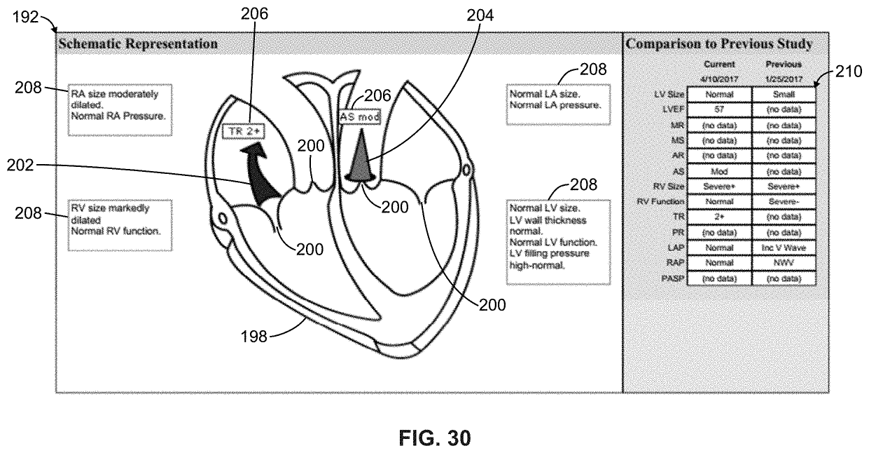

With reference to FIG. 30, a schematic representation 192 from report 134 is presented. This schematic representation 192 shows a moderate aortic stenosis and 2+ tricuspid regurgitation. This finding would not invoke the immediate contact requirement associated with a critical finding.

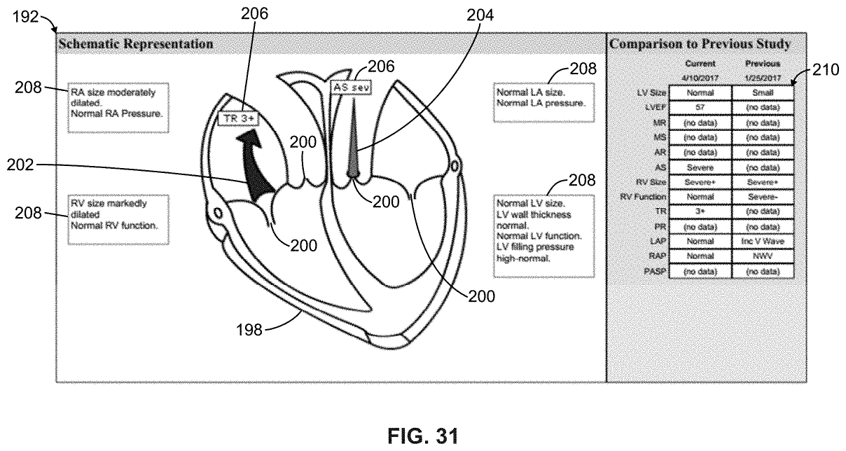

With reference to FIG. 31, a schematic representation 192 from report 134 is presented. This schematic representation 192 shows a severe aortic stenosis and 3+ tricuspid regurgitation. This critical finding would invoke the immediate contact requirement associated with a critical finding.

Example of Concordant Data:

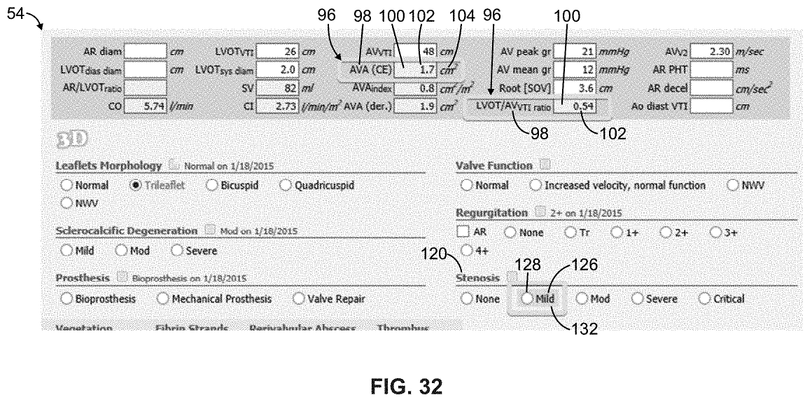

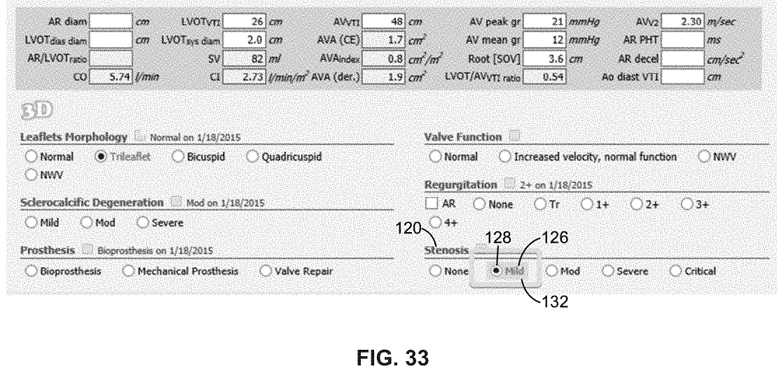

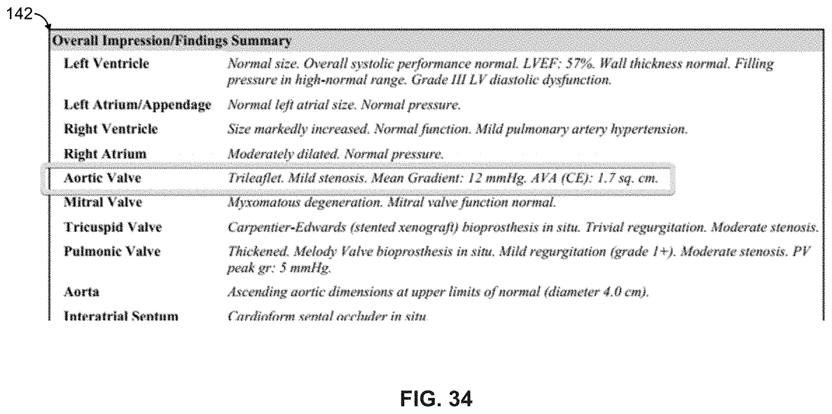

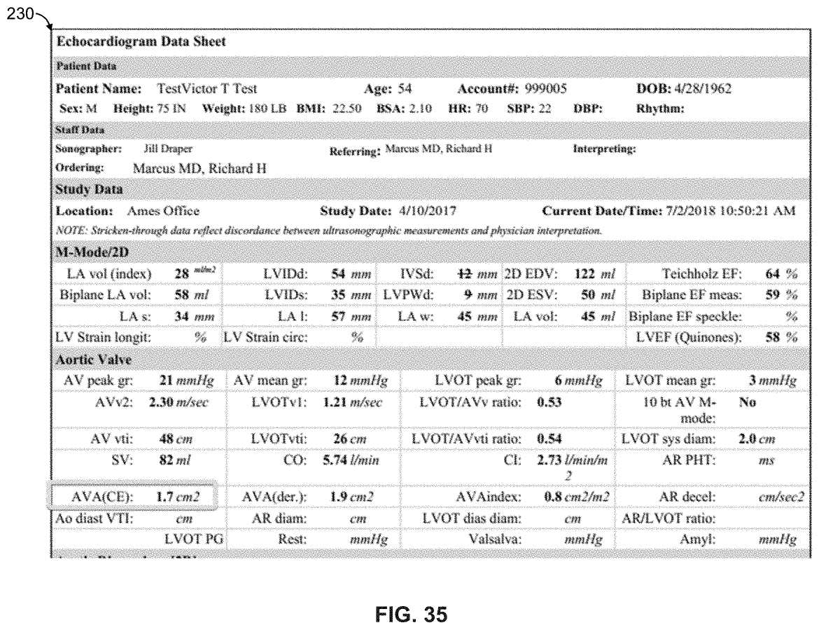

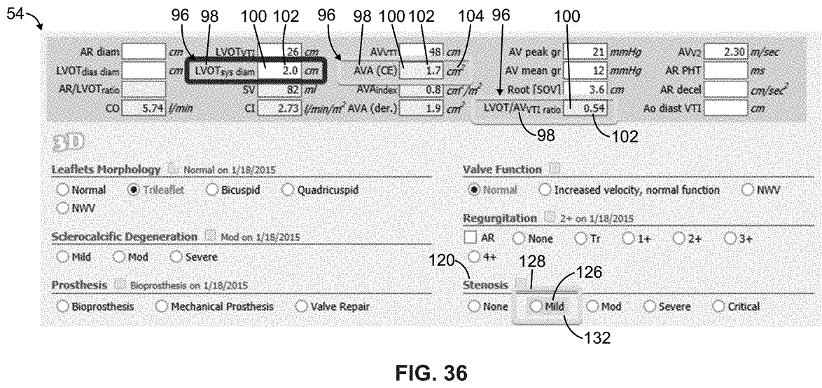

With reference to FIG. 32, a portion of interactive user page 54 associated with aortic valve tab 154 is presented. This figure illuminates two fields 96 “AVA (CE) 1.7 cm²” (which individually correlates with a “Mild” determination for “Stenosis” descriptor 120) and “LVOT/AV_{VTI} ratio 0.54” (which individually correlates with a “Mild” determination for “Stenosis” descriptor 120) which based on applicable standards of interpreting this information system 10 collectively suggests a “Mild” determination. As such, as is also shown, a suggested finding indicator 132 is placed around the “Mild” descriptor 126 under the “Stenosis” descriptor 120. FIG. 33 shows cardiologist 18 selecting the input area 128 associated with a “Mild” determination. As such, this is an example where the data (“AVA (CE)” and “LVOT/AV_{VTI} ratio”) are concordant with one another, and the determination of cardiologist 18 is concordant with the values in current data set 26. FIG. 34 shows a portion of overall impression 142 associated with FIG. 32 and FIG. 33. As the determination of cardiologist 18 is concordant with the expected determination of the system 10 based on the values 102 in current data set 26, the value 102 “AVA (CE) 1.7 sq. cm.” is allowed to appear in overall impression 142 of report 134. Similarly, as the determination of cardiologist 18 is concordant with the expected determination of the system 10 based on the values 102 in current data set 26, with reference to FIG. 35, the value 102 “AVA (CE) 1.7 cm²” is allowed to appear in data sheet 230 of report 134 in a normal manner (meaning that there is no strikethrough associated with this value).

Example of Discordant Data:

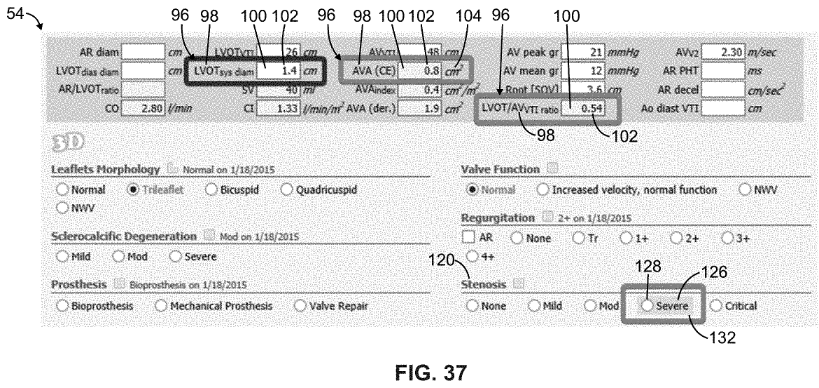

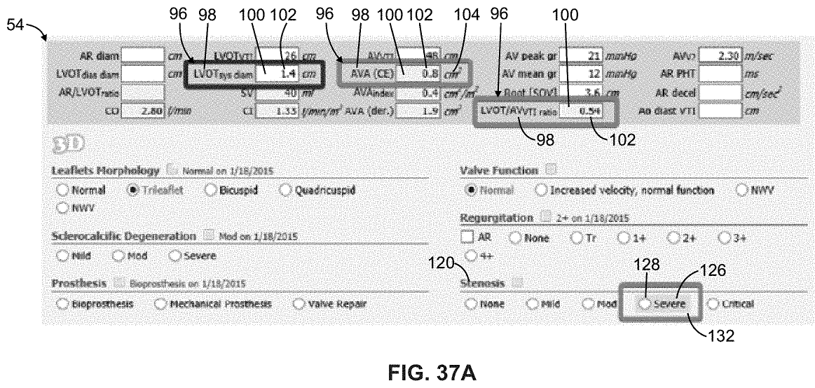

With reference to FIG. 37, a portion of interactive user page 54 associated with aortic valve tab 154 is presented.

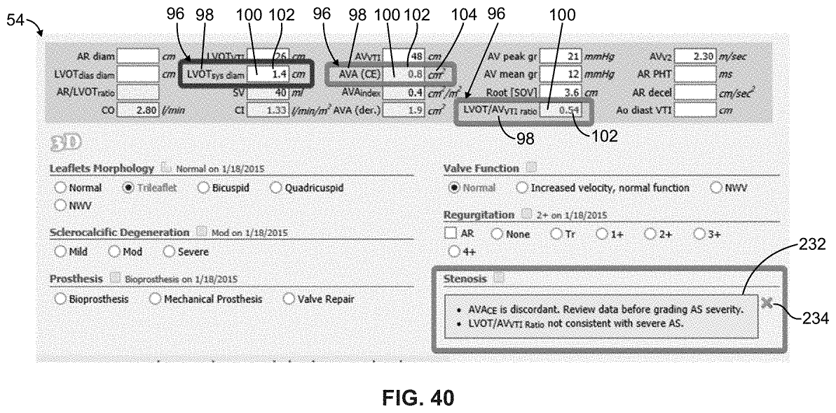

FIG. 37, shows the same portion of interactive user page 54 associated with aortic valve tab 154 as shown in FIG. 32, however this figure shows different values 102 in the fields 96 “LVOT_{sys diam} 1.4 cm,” “AVA (CE) 0.8 cm²” (which individually correlates with a “Severe” determination for “Stenosis” descriptor 120) and the same value 102 in the field 96 “LVOT/AV_{VTI} ratio 0.54” (which individually correlates with a “Mild” determination for “Stenosis” descriptor 120). Based on applicable standards of interpreting this information system 10 determines that the inputs are in conflict with one another, or said another way are discordant with one another. One input suggests “Mild” stenosis and the other input suggests “Severe” stenosis. When this discordant data situation is detected, in one arrangement, system 10 preemptively informs cardiologist 18 of the discordance of these inputs by presenting a discordant selection indicator 232 (shown in FIG. 40). In the arrangement shown, as one example, discordant selection indicator 232 is presented over the top of and thereby blocks access to the descriptor 120 and selection of evaluation options 124. This serves as an unavoidable pre-warning to cardiologist 18 of the discordant data. To gain access to the evaluation options 124, cardiologist 18 must clear the discordant selection indicator 232 by selecting the close marker 234. Thereafter, cardiologist 18 may select their determination with full understanding that the inputs are discordant.

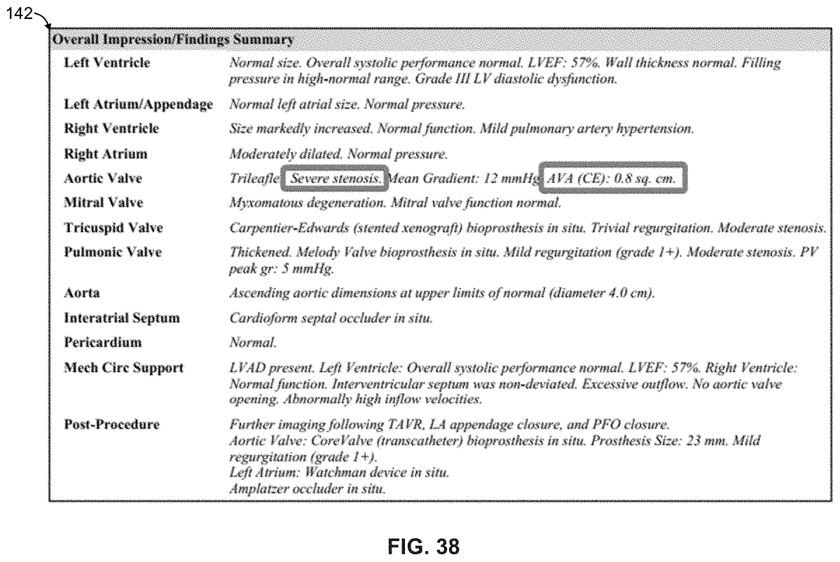

FIG. 38 shows a portion of overall impression 142 associated with FIG. 37 if cardiologist selects a “Severe” determination, which is concordant with the input for “AVA (CE) 0.8 cm²” which is why this value appears in the report 134 (whereas the discordant value, “LVOT/AV_{VTI} ratio 0.54” is omitted from report 134).

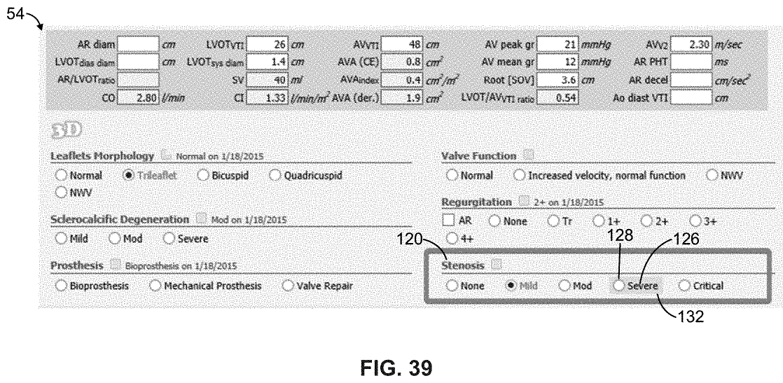

In contrast, with reference to FIG. 39, while the same values 102 are present in this figure as they are in FIG. 37 (“LVOT_{sys diam} 1.4 cm,” “AVA (CE) 0.8 cm²” and “LVOT/AV_{VTI} ratio 0.54.”) if cardiologist 18 selects the “Mild” determination for stenosis, the concordant data “LVOT/AV_{VTI} ratio 0.54” appears in the report 134 (whereas the discordant value, “AVA (CE) 0.8 cm²” is omitted from report 134). Discordant selection indicator 232 immediately informs cardiologist of the discordance and may be presented preemptively when the data are discordant with one another or in response to when cardiologist 18 makes a selection that is discordant with the data. As is shown, a close marker 234 is presented on discordant selection indicator 232 that allows cardiologist 18 to actively close or dismiss the discordant selection indicator 232 when it appears. However, this is only after cardiologist 18 has been forced to consider and then actively dismiss the discordance. This essentially allows the judgment of cardiologist 18 to trump the judgment of system 10. This process has a drastic effect of limiting entry errors by alerting cardiologist 18 to the discordance. This process also has the effect of allowing cardiologist 18 to spend more time considering discordant data and/or determinations and less time considering concordant data and/or determinations.

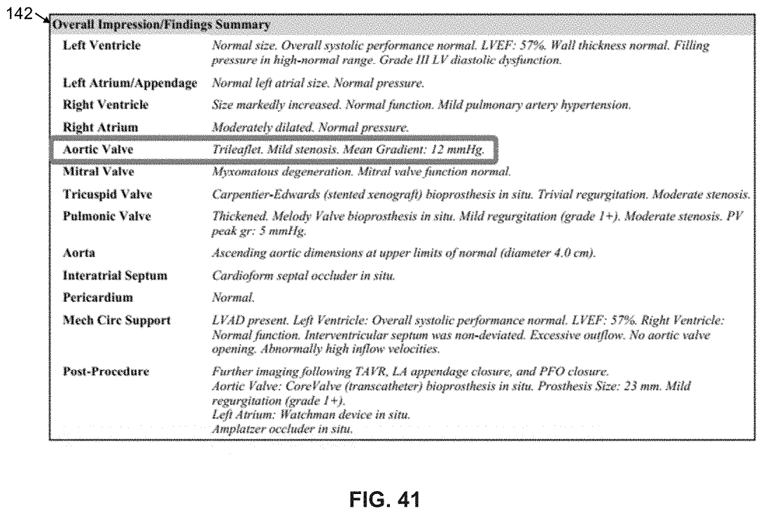

FIG. 41 shows a portion of overall impression 142 associated with FIG. 40. As the determination of cardiologist 18 is discordant with the expected determination of the system 10 based on the values 102 in current data set 26, the value 102 “AVA (CE) 0.8 sq. cm.” does not appear in overall impression 142 of report 134 as this would be in-conflict with the determination of “Mild stenosis”. In this way, system 10 prevents conflicting information from entering the report 134, and instead allows the judgment of cardiologist 18 to trump the suggestions of system 10. This difference can be seen by comparing the concordant overall impression 142 of FIG. 38 which includes the value 102 “AVA (CE) 0.8 sq. cm.” to the discordant overall impression 142 of FIG. 41 which does not includes the value 102 “AVA (CE) 0.8 sq. cm.”

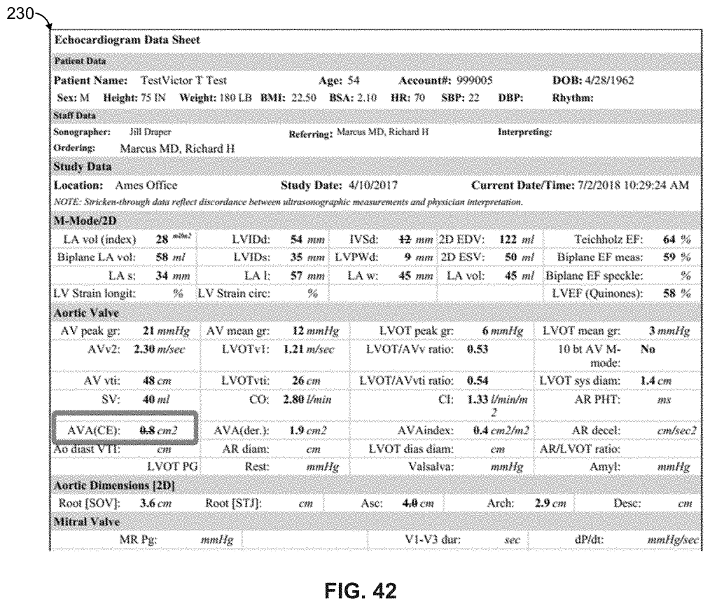

As the determination of cardiologist 18 is discordant with the expected determination of the system 10 based on the values 102 in current data set 26, with reference to FIG. 42, the value 102 “AVA (CE) cm²” appears in data sheet 230 of report 134 in a strikethrough text. This strikethrough text indicates that discordance exists and that cardiologist 18 has essentially ruled against this value, or brought this value into question. This indication allows a later reviewer to review the data sheet 230 and report 134 with a higher level of confidence and understanding without the confusion and conflict of discordant information and findings.

Another Example of Discordant Data:

With reference to FIG. 44, interactive user page 54 associated with left ventricle tab 112 is presented. This figure shows that based on the values 102 in data section 114 based on applicable standards of interpreting this information system 10 suggests a “Normal” determination for the evaluation section 118 “Left Ventricle Cavity Size”. As such, as is also shown, a suggested finding indicator 132 is placed around the “Normal” descriptor 126 under the “Left Ventricle Cavity Size” descriptor 120 for this evaluation section 118. In addition, in this figure, it is shown that cardiologist 18 has selected the input area 128 for a “Normal” determination. As such, the determination of cardiologist 18 is concordant with interpretation of the values 102 in data section 114 based on applicable standards.

With reference to FIG. 45, a section of data sheet 230 of report 134 is shown that corresponds to the selections made in FIG. 44. In this portion of data sheet 230 of report 134 it is shown that “VIDd: 50 mm” is present in the report without any indications, text treatment or strike-through text, which means to a reviewer that this value 102 can be relied upon and has not been determined to be discordant with the findings or determinations of cardiologist 18.

With reference to FIG. 46, interactive user page 54 associated with left ventricle tab 112 is presented that is identical to that shown in FIG. 44, with the difference being that cardiologist 18 has selected a “Mild+” determination for the evaluation section 118 “Left Ventricle Cavity Size”. The determination of cardiologist 18 is discordant with the suggestion of system 10, as is shown by the placement of a suggested finding indicator 132 around the “Normal” descriptor 126 under the “Left Ventricle Cavity Size” descriptor 120 for this evaluation section 118. In one arrangement, when cardiologist 18 makes this discordant determination a discordant selection indicator 232 is presented to cardiologist 18 which must be dismissed by selecting a close marker 234.

With reference to FIG. 47, a section of data sheet 230 of report 134 is shown that corresponds to the selections made in FIG. 46. In this portion of data sheet 230 of report 134 it is shown that “VIDd: *50 mm” is present in the report. In addition, in the header of data sheet 230 of report 134 it states: “NOTE: Data marked with * reflect discordance between ultrasonographic measurements and physician interpretation.” As such, the presence of the asterisk in “VIDd: *50 mm” in data sheet 230 of report 134 indicates a discordance. In an alternative arrangement, this could be strikethrough text treatment.

In Operation:

As one example, system 10 is used by treating physician 14 ordering patient 12 to undergo an echocardiogram. Echo tech 16 using echocardiogram testing device 20 having a computing device 22, an interactive user display 24, a processor 32, memory 34 and software 36 performs an echocardiogram on patient 12 and in doing so, echo tech 16 collects current data set 26 which includes a plurality of values 102. Current data set 26 may be stored in memory 34 of computing device 22 of echocardiogram testing device 20, which may be on board or at a remote location. In one arrangement, current data set 26 is transmitted over an electronic network, such as an intranet, the internet or another network, to database 40 and/or server of cloud 42 for later retrieval, review, manipulation and processing.

After the performance of the echocardiogram on patient 12 by echo tech 16, cardiologist 18 reviews the results, current data set 26 using system 10. Cardiologist 18, using computing device 44 having a processor 46, memory 48, software 50 and interactive user display 52 logs into system 10. Upon doing so, cardiologist 18 is presented with patient search page 62. Cardiologist 18 selects patient 12 from the list of patients 66 or by using search function 74.

Once patient 12 has been selected, system 10, using computing device 22, processor 32, memory 34 and software 36 retrieves current data set from database 40 and/or server or cloud 42 and auto populates values 102 from current data set into input areas 100, performs calculations for calculated values, generates suggested finding indicators 132 and performs countless other operations.

After cardiologist 18 selects patient 12 interactive user display 52 of computing device 44 displays interactive user pages 54 that display current data set 26 of patient 12. Along the left side of interactive user pages 54 is a list of tabs 56, each tab directed to a subset or category of current data set 26. Each tab 56 includes a parameter 78 that identifies the tab 56 and a progress bar 80 that indicates the amount of completion performed on that particular tab 56 which aids cardiologist 18 in their review of current data set 26 as they navigate through the interactive user pages 54 of system 10. Along the top of interactive user pages 54 is a menu tab 82. Through the use of tabs 56 and menu tabs 82, cardiologist 18 may navigate through system 10 while completing their review of current data set 26.

After selecting patient 12, cardiologist 18 is initially brought to the interactive user page 54 associated with demographics tab 84, where cardiologist 18 reviews the demographic data associated with patient 12. Thereafter, cardiologist 18 may navigate through and complete the interactive user pages 54 associated with the tabs 56: data sheet tab 106, left ventricle tab 112, left atrium tab 148, right ventricle tab 150, right atrium tab 152, aortic valve tab 154, mitral valve tab 156, tricuspid valve tab 158, pulmonic valve tab 160, aorta tab 162, pericardium tab 164, interatrial septum tab 166, pulmonary artery tab 168, extracardiac tab 170, mechanical circulation support tab 172, and post-procedure tab 174. As cardiologist 18 navigates among these tabs 56 cardiologists 18 reviews the values 102 of data sections 114 and makes their determinations by making their selections in evaluation sections 118.

When making their determinations, cardiologist 18 is aided by indications when a value 102 of current data set 26 is outside a predetermined amount from prior data set 28. When making their determinations, cardiologist 18 is aided by indications when a value 102 of current data set 26 is calculated from other values 102 in current data set 26. When making their determinations, cardiologist 18 is aided by suggested finding indicators 132 positioned around the suggested descriptor 126 of the suggested evaluation option 124, which is the suggested determination generated by system 10 using applicable standards based upon values 102 of the current data set 26. When making their determinations, cardiologist 18 is aided by mandatory finding indicators 130 which indicate when a determination is mandatory for an evaluation section 118. When making their determinations, cardiologist 18 is aided by an efficient layout for evaluation sections 118 which include a plurality of predetermined evaluation options 124 that can be quickly and easily selected.

As cardiologist 18 works their way through the interactive user pages 54, the progress bar 80 in tabs 56 indicates the amount of progress made and the amount of progress yet to be made. This aids cardiologist 18 to efficiently navigate through interactive user pages 54. As cardiologist 18 works their way through the interactive user pages 54, cardiologist 18 may take notes in comments sections 95 of the interactive user pages 54, which will be included in report 134. In addition, based on the determinations made by cardiologist 18, system 10 automatically generates comments in summary section 136 including summary comments in overall impression section 138 and more detailed comments in detailed finding sections 140.

When making their determinations, in the event that cardiologist 18 makes a selection that is discordant with the suggestions of system 10, as is indicated by a suggested finding indicator 132, cardiologist 18 is presented with a discordant selection indicator 232. Upon encountering a discordant selection indicator 232, cardiologist 18 is forced to consider and/or reconsider their discordant selection as the cardiologist must select the close marker 234.

At any time during their evaluation, cardiologist 18 may select the Preview Report menu tab 82 to preview the report 134 to ensure their determinations are correct and that the report 134 appears as they desire. Once cardiologist 18 makes all of their required determinations as indicated by mandatory finding indicators 130 and completed progress bars 80, cardiologist selects the Finalize menu tab 82 at which point report 134 is generated. Notably, system 10 prevents cardiologist 18 from finalizing the report 134 until all mandatory determinations have been made, thereby preventing the submission of an incomplete report 134.

Once finalized, report 134 is distributed to the treating physician 14 and other contacts (as is indicated on demographics tab 84). When report 134 includes a critical finding, cardiologist 18 is prompted to immediately contact treating physician 14, patient 12 or another person that provides care for patient 12 to ensure that proper care is being given to patient 12 in view of the findings of cardiologist 18.

Customization:

It is to be understood that the examples shown in the figures are merely examples. Any other layout or arrangement is hereby contemplated for use for interactive user pages 54 as well as the content of those interactive user pages 54.

LAA Occlusion Device:

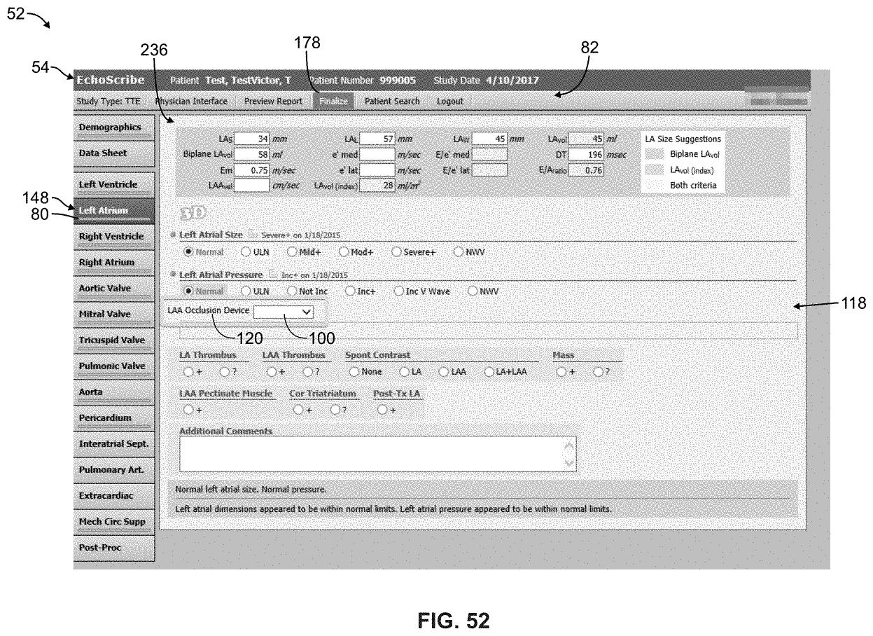

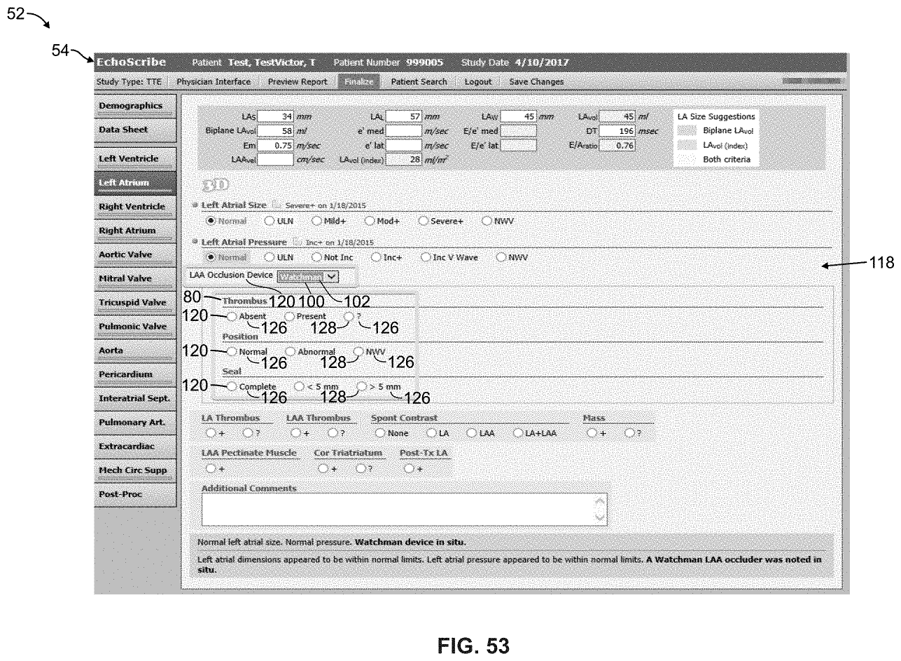

With reference to FIG. 52 and FIG. 53, as an example of the customization capabilities for system 10 and the ability for system 10 to be continually improved to reflect the present needs of cardiologists 18 and developments in cardiology, FIG. 52 shows in highlighted form evaluation section 118 directed to “LAA Occlusion Device” as descriptor 120 which includes a drop-down box as input area 100. This drop down box of input area 100, as is populated with potential LAA Occlusion Devices, which presently includes the “Watchman” device and the “Lariat” device, however any other device may be included thereunder. When an LAA Occlusion Device is selected, as is shown, in FIG. 53 where a Watchman device has been selected under input area 100, a sub-group of further selections are presented that specifically relate to the Watchman device. These further selections may be parameters that are provided by the manufacture of the LAA Occlusion Device that must be periodically monitored. By incorporating these selections into the system 10 and presenting them as sub-group determinations this relieves the cardiologist 18 of the need to remember this information, and/or relives the cardiologist 18 of the need to look up information about what needs to be monitored or what steps must be taken when encountering one of these devices in an echocardiogram. In the arrangement shown, as one example, with reference to FIG. 53, sub-groups of information include a “Thrombus” descriptor 120 with “Absent” “Present” and “?” as descriptors 126 with associated input areas 128; a “Position” descriptor 120 with “Normal” “Abnormal” and “NWV” as descriptors 126 with associated input areas 128; and a “Seal” descriptor 120 with “Complete” “<5 mm” and “>5 mm” as descriptors 126 with associated input areas 128.

In this way, the inclusion of this information ensures cardiologist 18 is aware of the need to review the results of echocardiogram for these findings, which may be made as mandatory findings when applicable thereby ensuring they are considered and determined. This has the tendency to improve the accuracy and thoroughness of the review and the report 134.

Overall Improvement to Electronic Health Records (EHR):

The system 10 presented herein is a substantial improvement to the current state-of and mandate-for electronic health records. While the purpose of EHRs are noble, in practice EHR rules, requirements and mandates are becoming overly burdensome, bureaucratic and actually get in the way of providing care to patient 12 instead of helping provide care to patient 12. However, the system 10 presented herein is a first example of where an EHR actually makes physicians more efficient, with less errors and omissions, while providing a better result (report 134). The EHR presented herein helps to guide cardiologist 18 through the process of evaluating an echocardiogram, and provides a report 134 at the end of the process that can be relied upon with a higher level of certainty than any other previously existing EHR.

Not Limited to Echocardiograms:

While reference is made to echocardiograms, as well as cardiologist 18, the disclosure herein is not limited to echocardiograms. Instead, reference to echocardiograms is only one of countless examples of how the system 10 presented herein may be used. It is hereby contemplated for use that system 10 may be used in association with any medical procedure other than echocardiograms to help speed the review of the test or procedure, improve the results, avoid entry errors, and eliminate discordant and confusing information in reports, among other purposes.

From the above discussion, it will be appreciated that the system and method of interpreting an echocardiogram presented herein improves upon the state of the art.

Specifically, the system and method of interpreting an echocardiogram: provides a healthcare professional with clean, organized and easy to understand information which leads to more effective patient care; is an efficient and user friendly diagnosing tool; provides information in a quickly understandable format; provides a healthcare professional with a tool that displays the parameters related to the bounds of cardiovascular disease diagnosis; presents a digestible amount of the correct information needed for specific; provides highlighted fields of a suggested interpretation and/or diagnosis; alerts a healthcare professional to varying data from previous tests; provides critical finding alerts; precludes careless and/or mistaken data entry; highlights discordant data; prompts a healthcare professional to note a reason for discordant data; provides the ability for a healthcare professional to change or amend data provided by an echocardiogram; creates digestible reports; creates comparable reports; provides a means for sharing information with others; helps the healthcare professional effectively treat patients; produces more-consistent; provides advice to a healthcare professional on selection of statuses, and or any other information needed or used in concurrence with diagnosing, treating, and or evaluating; provides a visual means for viewing a patient's history; incorporates in-line information related to a patient's history; provides an echocardiogram interpretation collated with historical information; incorporates historical information with current information to assist diagnosis; provides a healthcare professional with a visual tool to the patient's evaluation; provides an evidence-based management system for healthcare professionals; provides a means for healthcare professionals to produce objective based interpretations; provides a means for healthcare professionals to ensure they are meeting Intersocietal Accreditation Commission (IAC) guidelines; provides more consistent interpretations of echocardiograms, among countless other improvements and benefits.

It will be appreciated by those skilled in the art that other various modifications could be made to the system without parting from the spirit and scope of the disclosure. All such modifications and changes fall within the scope of the claims and are intended to be covered thereby.

What is claimed:

1. A method of interpreting an echocardiogram, the steps comprising:
receiving a current data set having a plurality of values from the performance of an echocardiogram on a patient;
receiving a prior data set having a plurality of values from the performance of an echocardiogram on a the patient;
storing the current data set in memory of a computing device;
storing the prior data set in memory of the computing device;
placing the current data set into a plurality of fields on an interactive user page using a processor and software on the computing device;
comparing the values of the current data set with values of the prior data set;
indicating when a value of the current data set is outside a predetermined variance from that value in the prior data set.

2. The method of claim 1, wherein indicating when a value of the current data set is outside a predetermined variance is selected from the group consisting of bolding the value, highlighting the value, italicizing the value, underlining the value, using a different font for the value, using a different color for the value, providing a box around the value, causing the value to flash, pulsate or strobe, providing asterisks or other text in association with the value, and providing a note with the value.

3. The method of claim 1, wherein the predetermined variance is ten percent or greater.

4. A method of interpreting an echocardiogram, the steps comprising:
receiving a current data set having a plurality of values from the performance of an echocardiogram on a patient;
storing the current data set in memory of a computing device;
placing the values of the current data set into a fields on an interactive user page using a processor and software on the computing device;
providing a selectable listing of a plurality of evaluation options on the interactive user page;
determining a suggested evaluation option of the plurality of evaluation options using predetermined guidelines applied to values of the current data using the processor and the software;
indicating the suggested evaluation option on the interactive user page as a recommended determination for selection by a cardiologist.

5. The method of claim 4, wherein indicating the suggested evaluation option on the interactive user page is selected from the group consisting of bolding the evaluation option, highlighting the evaluation option, italicizing the evaluation option, underlining the evaluation option, using a different font for the evaluation option, using a different color for the evaluation option, providing a box around the evaluation option, causing the value to evaluation option, pulsate or strobe, providing asterisks or other text in association with the evaluation option, and providing a note with the evaluation option.

6. The method of claim 4, further comprising the step of selecting an evaluation option by a cardiologist.

7. The method of claim 4. further comprising the step of providing a discordant selection indicator when a cardiologist selects an evaluation option other than the suggested evaluation option thereby.

8. A method of interpreting an echocardiogram, the steps comprising:
receiving a current data set having a plurality of values from the performance of an echocardiogram on a patient;
storing the current data set in memory of a computing device;
placing the values of the current data set into a fields on an interactive user page using a processor and software on the computing device;
providing a selectable listing of a plurality of evaluation options on the interactive user page;
determining a suggested evaluation option of the plurality of evaluation options using predetermined guidelines applied to values of the current data using the processor and the software;
selecting an evaluation option by a cardiologist;
providing a discordant selection indicator when the cardiologist selects an evaluation option other than the suggested evaluation option thereby.

9. The method of claim 8, further comprising the step of indicating the suggested evaluation option on the interactive user page as a recommended determination for selection by a cardiologist.

10. The method of claim 8, further comprising the step of indicating the suggested evaluation option on the interactive user page as a recommended determination for selection by a cardiologist, wherein indicating the suggested evaluation option on the interactive user page is selected from the group consisting of bolding the evaluation option, highlighting the evaluation option, italicizing the evaluation option, underlining the evaluation option, using a different font for the evaluation option, using a different color for the evaluation option, providing a box around the evaluation option, causing the value to evaluation option, pulsate or strobe, providing asterisks or other text in association with the evaluation option, and providing a note with the evaluation option.

11. The method of claim 8, wherein the discordant selection indicator is a popup box.

12. The method of claim 8, wherein the discordant selection indicator is a popup box that must be dismissed by the cardiologist to proceed.

13. A method of interpreting an echocardiogram, the steps comprising:
receiving a current data set having a plurality of values from the performance of an echocardiogram on a patient;
receiving at least one prior data set having a plurality of values from the performance of an echocardiogram on the patient;
storing the current data set in memory of a computing device;
storing the prior data set in memory of the computing device;
placing the values of the current data set into a plurality of fields on an interactive user page using a processor and software on the computing device;
displaying the at least one prior result of a field using a hover function when a cardiologist hovers over the field.

14. The method of claim 13, wherein the hover function is a popup box.

15. The method of claim 13, wherein the hover function is a popup box that includes at least one value from the at least one prior data set as well as the date of the at least one prior data set.

16. The method of claim 13, wherein the field includes a descriptor, an input area and a value.

17. A method of interpreting an echocardiogram, the steps comprising:
receiving a current data set having a plurality of values from the performance of an echocardiogram on a patient;
storing the current data set in memory of a computing device;
placing the values of the current data set into a plurality of fields on an interactive user page using a processor and software on the computing device;
providing a selectable listing of a plurality of evaluation options for selection by a cardiologist;
displaying a previously selected evaluation option in association with the plurality of evaluation options.

18. The method of claim 17, further comprising the step of displaying a plurality of previously selected evaluation options when the cardiologist hovers over the previously selected evaluation option using a hover function.

19. The method of claim 17, further comprising the step of displaying a plurality of previously selected evaluation options when the cardiologist hovers over the previously selected evaluation option using a hover function, wherein the hover function is a popup box.

20. The method of claim 17, further comprising the step of displaying a plurality of previously selected evaluation options when the cardiologist hovers over the previously selected evaluation option using a hover function, wherein the hover function is a popup box that includes a date and the previously selected evaluation options.
